(19) United States
(12) Patent Application Publication
(10) Pub. No.: US 2022/0143006 A1
(43) Pub. Date: May 12, 2022

(54) METHODS OF TREATING CANCER WITH FARNESYLTRANSFERASE INHIBITORS

(71) Applicant: Kura Oncology, Inc., San Diego, CA (US)

(72) Inventor: Antonio Gualberto, Boston, MA (US)

(21) Appl. No.: 17/438,893

(22) PCT Filed: Mar. 12, 2020
(86) PCT No.: PCT/US2020/022236

§ 371 Date: Sep. 13, 2021

Related U.S. Application Data
(60) Provisional application No. 62/819,407, filed on Mar. 15, 2019.
(60) Provisional application No. 62/860,685, filed on Jun. 12, 2019.

(51) Int. Cl. A61K 31/4709; A61K 45/06; A61K 38/50; A61P 35/00; C12Q 1/6886

(52) U.S. Cl. CPC A61K 31/4709; A61K 45/06; A61K 38/50; C12Q 2600/112; C12Q 1/6886; C12Q 2600/106; C12Q 2600/156; A61P 35/00

(57) ABSTRACT

The present invention relates to the field of molecular biology and cancer biology. Specifically, the present invention relates to methods of treating a KIR-mutant cancer in a subject with a farnesyltransferase inhibitor (FTI). The present invention also relates to methods of treating a subject with a farnesyltransferase inhibitor (FTI) that include determining whether the subject is likely to be responsive to the FTI treatment based on the mutation status of a member of the KIR family in the subject.

### CROSS REFERENCE

This application claims the benefit of priority from U.S. Provisional Application No. 62/819,407, filed on Mar. 15, 2019, and further claims the benefit of priority from U.S. Provisional Application No. 62/860,685, filed on Jun. 12, 2019. Each of the foregoing related applications, in its entirety, is incorporated herein by reference.

### FIELD

The present invention relates to the field of cancer therapy. In particular, provided are methods of treating cancer, with farnesyltransferase inhibitors.

### BACKGROUND

Stratification of patient populations to improve therapeutic response rate is increasingly valuable in the clinical management of cancer patients. Farnesyltransferase inhibitors (FTI) are therapeutic agents that have utility in the treatment of cancers, such as treatment of hematological or hematopoietic cancers, such as lymphoma (e.g., T-cell lymphoma, peripheral T-cell lymphoma (“PTCL”), natural killer cell lymphoma (“NK lymphoma”), cutaneous T-Cell lymphoma (“CTCL”), or angioimmunoblastic T-cell lymphoma (“AITL”)), leukemia (e.g., acute myeloid leukemia (AML), chronic myelogenous leukemia (CML)), and myelodysplastic syndromes (MDS)/myeloproliferative neoplasms (MPN) (e.g., chronic myelomonocytic leukemia (CMML), juvenile myelomonocytic leukemia (JMML)). However, patients respond differently to an FTI treatment. Therefore, methods to predict the responsiveness of a subject having cancer to an FTI treatment, or methods to select cancer patients for an FTI treatment, represent unmet needs. The methods and compositions provided herein meet these needs and provide other related advantages.

### SUMMARY

Provided herein are methods of treating a cancer in a subject (e.g., a human) comprising administering a farnesyltransferase inhibitor (FTI) to the subject, wherein the cancer is a cancer known to have or determined to have a mutation in a member of the KIR family. Provided herein are also methods to predict the responsiveness of a subject having cancer for an FTI treatment, methods to select a cancer patient for an FTI treatment, methods to stratify cancer patients for an FTI treatment, and methods to increase the responsiveness of a cancer patient population for an FTI treatment. In some embodiments, the methods include analyzing a sample from the subject having cancer to determining that the subject has KIR-mutant cancer prior to administering the FTI to the subject. In some embodiments, the FTI is tipifarnib. In specific embodiments, the cancer is hematological (or hematogenous) cancer (e.g., leukemia, lymphoma, myeloproliferative neoplasm (MPN), or myelodysplastic syndrome (MDS)) or a solid tumor.

In some embodiments, provided herein are methods of treating a cancer in a subject in need thereof, said method comprising administering a therapeutically effective amount of an FTI to said subject, wherein the cancer is a cancer known to have or determined to have a mutation in a member of the KIR2DL family and/or KIR3DL family.

Provided herein are methods of treating a cancer in a subject in need thereof, said method comprising administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2. Provided herein are also methods to predict the responsiveness of a subject having cancer for an FTI treatment, methods to select a cancer patient for an FTI treatment, methods to stratify cancer patients for an FTI treatment, and methods to increase the responsiveness of a cancer patient population for an FTI treatment. In some embodiments, the methods include analyzing a sample from the subject having cancer to determining that the subject has KIR-mutant cancer prior to administering the FTI to the subject. In some embodiments, the method further includes determining a KIR-mutant cancer variant allele frequency (VAF) in a sample from the cancer subject, wherein the KIR-mutant cancer is selected from the group consisting of: a KIR2DL1-mutant, a KIR2DL3-mutant, a KIR2DL4-mutant, a KIR3DL1-mutant, and/or a KIR3DL2-mutant. In some embodiments, the method further provides determining the VAF of a KIR3DL2 mutation from the sample from the cancer subject. In some embodiments, the method further provides determining the VAF of the KIR3DL2 mutation selected from the group consisting of: a KIR3DL2 C336R mutation, a KIR3DL2 Q386E mutation, or a KIR3DL2 C336R/Q386E mutation, from the sample from the cancer subject. In some embodiments, the FTI is tipifarnib. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is cutaneous T-Cell lymphoma (CTCL). In specific embodiments, the cancer is relapsed or refractory PTCL. In specific embodiments, the cancer is PTCL not otherwise specified (PTCL-NOS). In specific embodiments, the cancer is relapsed or refractory AITL. In specific embodiments, the cancer is AITL not otherwise specified (AITL-NOS). In specific embodiments, the cancer is anaplastic large cell lymphoma (ALCL)—anaplastic lymphoma kinase (ALK) positive. In specific embodiments, the cancer is anaplastic large cell lymphoma (ALCL)—anaplastic lymphoma kinase (ALK) negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is natural killer cell leukemia (NK leukemia). In specific embodiments, the cancer is AML. In specific embodiments, the leukemia is T-cell acute lymphoblastic leukemia (T-ALL). In specific embodiments, the cancer is CML. In specific embodiments, the cancer is MDS. In specific embodiments, the cancer is MPN. In specific embodiments, the cancer is CMML. In specific embodiments, the cancer is JMML.

Provided herein are methods of treating a cancer in a subject in need thereof, said method comprising administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, and wherein the KIR2DL1, KIR2DL3, KIR2DL4, KIR3DL1, and/or KIR3DL2, has two of more mutations comprising two or more modifications at two or more codons that encode two or more amino acids in the extracellular domain, at two or more codons that encode two or more amino acids in the cytoplasmic domain, or combinations thereof.

Provided herein are methods of treating a cancer in a subject in need thereof, said method comprising administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, and wherein the KIR2DL1, KIR2DL3, KIR2DL4, KIR3DL1, and/or KIR3DL2, has three of more mutations comprising three or more modifications at three or more codons that encode three or more amino acids in the extracellular domain, at three or more codons that encode three or more amino acids in the cytoplasmic domain, or combinations thereof.

Provided herein are methods of treating a cancer in a subject in need thereof, said method comprising administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, and wherein the KIR2DL1, KIR2DL3, KIR2DL4, KIR3DL1, and/or KIR3DL2, has four of more mutations comprising four or more modifications at four or more codons that encode four or more amino acids in the extracellular domain, at four or more codons that encode four or more amino acids in the cytoplasmic domain, or combinations thereof.

Provided herein are methods of treating a cancer in a subject in need thereof, said method comprising administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, and wherein the KIR-mutant cancer is a cancer known to have or determined to have a mutation in two, three, four, or each of the members of the KIR family selected from the group consisting of KIR2DL1, KIR2DL3, KIR2DL4, KIR3DL1, and KIR3DL2.

Provided herein are methods of treating a cancer in a subject in need thereof, said method comprising administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, such as two, three, four, or more mutations, in the KIR2DL1, KIR2DL3, KIR2DL4, KIR3DL1, and/or KIR3DL2.

In some embodiments, the member of the KIR family having or determined to have a mutation is a member of the KIR2DL family and/or KIR3DL family. In some embodiments, the KIR-mutant cancer is a cancer known to have or determined to have a mutation (e.g., two, three, four, or more mutations) in a member of the KIR2DL family selected from the group consisting of: KIR2DL1, KIR2DL3, and KIR2DL4. In some embodiments, the KIR-mutant cancer is a cancer known to have or determined to have a mutation (e.g., two, three, four, or more mutations) in a member of the KIR3DL family selected from the group consisting of: KIR3DL1 and KIR3DL2. In some embodiments, the KIR-mutant cancer is a cancer known to have or determined to have a mutation (e.g., two, three, four, or more mutations) in a member of the KIR2DL family and/or KIR3DL family selected from the group consisting of: KIR2DL1, KIR2DL3, KIR2DL4, KIR3DL1, and KIR3DL2.

In some embodiments, the methods provided herein include determining the presence of the mutation in the KIR2DL1, KIR2DL3, KIR2DL4, KIR3DL1, and/or KIR3DL2 (e.g., determining the presence of the two, three, four, or more mutations, in the KIR2DL1, KIR2DL3, KIR2DL4, KIR3DL1, and/or KIR3DL2) in a sample from a subject having cancer, and administering a therapeutically effective amount of an FTI to said subject if the mutation in the KIR2DL1, KIR2DL3, KIR2DL4, KIR3DL1, and/or KIR3DL2 is present (e.g., if the two, three, four, or more mutations, in the KIR2DL1, KIR2DL3, KIR2DL4, KIR3DL1, and/or KIR3DL2 are present). In some embodiments, the mutation in the KIR2DL1, KIR2DL3, KIR2DL4, KIR3DL1, and/or KIR3DL2 is or comprises a modification in a codon that encodes an amino acid in the extracellular domain, in the cytoplasmic domain, or combinations thereof. In some embodiments, the methods provided herein include determining the presence of has two, three, four, or more, mutations in the KIR2DL1, KIR2DL3, KIR2DL4, KIR3DL1, and/or KIR3DL2, comprising two, three, four, or more, modifications at two, three, four, or more, codons that endode two, three, four, or more, amino acids in the extracellular domain, at two, three, four, or more, codons that endode two, three, four, or more, amino acids in the cytoplasmic domain, or combinations thereof. Provided herein are also methods to predict the responsiveness of a subject having cancer for an FTI treatment, methods to select a cancer patient for an FTI treatment, methods to stratify cancer patients for an FTI treatment, and methods to increase the responsiveness of a cancer patient population for an FTI treatment. In some embodiments, the methods include analyzing a sample from the subject having cancer to determining that the subject has KIR-mutant cancer prior to administering the FTI to the subject. In some embodiments, the FTI is tipifarnib. In specific embodiments, the cancer is hematological (or hematogenous) cancer (e.g., leukemia, lymphoma, myeloproliferative neoplasm (MPN), or myelodysplastic syndrome (MDS)) or a solid tumor.

In some embodiments, provided herein are methods of treating a KIR-mutant cancer in a subject in need thereof, said method comprising administering a therapeutically effective amount of an FTI to said subject, wherein the KIR-mutant cancer is a cancer known to have or determined to have a mutation in a member of the KIR2DL family and/or KIR3DL family. In some embodiments, provided herein are methods of treating a KIR-mutant cancer in a subject in need thereof, said method comprising administering a therapeutically effective amount of an FTI to said subject, wherein the KIR-mutant cancer is a cancer known to have or determined to have a mutation in a member of the KIR family selected from the group consisting of KIR2DL1, KIR2DL3, KIR2DL4, KIR3DL1, and KIR3DL2. In some embodiments, the KIR2DL1, KIR2DL3, KIR2DL4, KIR3DL1, and/or KIR3DL2, has two of more mutations comprising two or more modifications at two or more codons that encode two or more amino acids in the extracellular domain, at two or more codons that encode two or more amino acids in the cytoplasmic domain, or combinations thereof. In some embodiments, the KIR2DL1, KIR2DL3, KIR2DL4, KIR3DL1, and/or KIR3DL2, has three of more mutations comprising three or more modifications at three or more codons that encode three or more amino acids in the extracellular domain, at three or more codons that encode three or more amino acids in the cytoplasmic domain, or combinations thereof. In some embodiments, the KIR-mutant cancer has or comprises a mutation in KIR2DL1. In some embodiments, the KIR-mutant cancer has or comprises a mutation in KIR2DL3. In some embodiments, the KIR-mutant cancer has or comprises a mutation in KIR2DL4. In some embodiments, the KIR-mutant cancer has or comprises a mutation in KIR3DL1. In some embodiments, the KIR-mutant cancer has or comprises a mutation in KIR3DL2. In some embodiments, the KIR-mutant cancer is a cancer known to have or determined to have a mutation in two or more members of the KIR family selected from the group consisting of KIR2DL1, KIR2DL3, KIR2DL4, KIR3DL1, and KIR3DL2. In some embodiments, the KIR-mutant cancer is a cancer known to have or determined to have a mutation in three or more members of the KIR family selected from the group consisting of KIR2DL1, KIR2DL3, KIR2DL4, KIR3DL1, and KIR3DL2. In some embodiments, the KIR-mutant cancer is a cancer known to have or determined to have a mutation in four or more members of the KIR family selected from the group consisting of KIR2DL1, KIR2DL3, KIR2DL4, KIR3DL1, and KIR3DL2. In some embodiments, the KIR-mutant cancer is a cancer known to have or determined to have a mutation in each of the members of the KIR family selected from the group consisting of KIR2DL1, KIR2DL3, KIR2DL4, KIR3DL1, and KIR3DL2.

Provided herein are methods of treating a cancer in a subject in need thereof, said method comprising administering a therapeutically effective amount of an FTI to said subject, wherein the cancer is a cancer known to have or determined to have a mutation in KIR2DL1, such as two, three, four, or more mutations, in KIR2DL1. In some embodiments, the methods provided herein include determining the presence of the mutation in KIR2DL1 (e.g., determining the presence of the two, three, four, or more mutations, in KIR2DL1) in a sample from a subject having cancer, and administering a therapeutically effective amount of an FTI to said subject if the mutation in KIR2DL1 is present (e.g., if the two, three, four, or more mutations, in KIR2DL1 are present). In some embodiments, the mutation is or comprises a modification in a codon of KIR2DL1 encoding an amino acid in the extracellular domain, in the cytoplasmic domain, or combinations thereof. In some embodiments, the mutation is or comprises a modification in a codon of KIR2DL1 encoding an amino acid in the extracellular domain, selected from a group consisting of: M65, H77, A83, S88, T91, L140, N178, G179, D184, R197, F202, and H203. In some embodiments, the mutation in the extracellular domain of KIR2DL1 is selected from a group consisting of: M65T, H77N, H77L, A83G, S88G, T91K, L140Q, N178D, G179R, D184N, R197T, F202L, and H203R. In some embodiments, the mutation is or comprises a modification in a codon of KIR2DL1 encoding an amino acid in the extracellular D2 domain selected from a group consisting of: N178, G179, D184, R197, and F202. In some embodiments, the mutation in the extracellular D2 domain of KIR2DL1 is selected from a group consisting of: N178D, G179R, D184N, R197T, and F202L. In specific embodiments, the cancer is hematological (or hematogenous) cancer (e.g., leukemia, lymphoma, myeloproliferative neoplasm (MPN), or myelodysplastic syndrome (MDS)) or a solid tumor.

Provided herein are methods of treating a cancer in a subject in need thereof, said method comprising administering a therapeutically effective amount of an FTI to said subject, wherein the cancer is a cancer known to have or determined to have a mutation in KIR2DL3, such as two, three, four, or more mutations, in KIR2DL3. In some embodiments, the methods provided herein include determining the presence of the mutation in KIR2DL3 (e.g., determining the presence of the two, three, four, or more mutations, in KIR2DL3) in a sample from a subject having cancer, and administering a therapeutically effective amount of an FTI to said subject if the mutation in KIR2DL3 is present (e.g., if the two, three, four, or more mutations, in KIR2DL3 are present). In some embodiments, the mutation is or comprises a modification in a codon of KIR2DL3 encoding an amino acid mutation is or comprises a modification in a codon of KIR2DL3 encoding an amino acid in the extracellular domain, in the cytoplasmic domain, or combinations thereof. In some embodiments, the mutation is or comprises a modification in a codon of KIR2DL3 encoding an amino acid mutation is or comprises a modification in a codon of KIR2DL3 encoding an amino acid selected from a group consisting of: F66, R162, R169, F171, S172, E295, R318, I330, I331, and V332. In some embodiments, the mutation in KIR2DL3 is selected from a group consisting of: F66Y, R162T, R169C, F171L, S172P, E295D, R318C, I330T, I331T, and V332M. In some embodiments, the mutation is or comprises a modification in a codon of KIR2DL3 encoding the amino acid R162 and/or E295. In some embodiments, the mutation in KIR2DL3 is or comprises the R162T and/or the E295D. In specific embodiments, the cancer is hematological (or hematogenous) cancer (e.g., leukemia, lymphoma, myeloproliferative neoplasm (MPN), or myelodysplastic syndrome (MDS)) or a solid tumor.

Provided herein are methods of treating a cancer in a subject in need thereof, said method comprising administering a therapeutically effective amount of an FTI to said subject, wherein the cancer is a cancer known to have or determined to have a mutation in KIR2DL4, such as two, three, four, or more mutations, in KIR2DL4. In some embodiments, the methods provided herein include determining the presence of the mutation in KIR2DL4 (e.g., determining the presence of the two, three, four, or more mutations, in KIR2DL4) in a sample from a subject having cancer, and administering a therapeutically effective amount of an FTI to said subject if the mutation in KIR2DL4 is present (e.g., if the two, three, four, or more mutations, in KIR2DL4 are present). In some embodiments, the mutation is or comprises a modification in a codon of KIR2DL4 encoding an amino acid in the extracellular domain, in the cytoplasmic domain, or combinations thereof. In some embodiments, the mutation is or comprises a modification in a codon of KIR2DL4 encoding an amino acid selected from a group consisting of: R50, H52, R55, N58, T61, K65, Q149, I154, E162, L166, I174, A238, and S267. In some embodiments, the mutation in KIR2DL4 is selected from a group consisting of: R50L, H52R, R55L, N58T, T61R, K65E, Q149K, Q149R, I154M, E162K, E162G, L166P, I174V, A238P, and S267fs. In some embodiments, the mutation is or comprises a modification in a codon of KIR2DL4 encoding the amino acid Q149 and/or I154 in the extracellular D2 domain. In some embodiments, the mutation in the extracellular D2 domain of KIR2DL4 is or comprises the Q149K, Q149R, and/or I154M. In specific embodiments, the cancer is hematological (or hematogenous) cancer (e.g., leukemia, lymphoma, myeloproliferative neoplasm (MPN), or myelodysplastic syndrome (MDS)) or a solid tumor.

Provided herein are methods of treating a cancer in a subject in need thereof, said method comprising administering a therapeutically effective amount of an FTI to said subject, wherein the cancer is a cancer known to have or determined to have a mutation in KIR3DL1, such as two, three, four, or more mutations, in KIR3DL1. In some embodiments, the methods provided herein include determining the presence of the mutation in KIR3DL1 (e.g., determining the presence of the two, three, four, or more mutations, in KIR3DL1) in a sample from a subject having cancer, and administering a therapeutically effective amount of an FTI to said subject if the mutation in KIR3DL1 is present (e.g., if the two, three, four, or more mutations, in KIR3DL1 are present). In some embodiments, the mutation is or comprises a modification in a codon of KIR3DL1 encoding an amino acid in the extracellular domain, in the cytoplasmic domain, or combinations thereof. In some embodiments, the mutation is or comprises a modification in a codon of KIR3DL1 encoding an amino acid selected from a group consisting of: R292, F297, P336, R409, R413, I426, L427, T429, and V440. In some embodiments, the mutation in KIR3DL1 is selected from a group consisting of: R292T, F297L, P336R, R409T, R413C, I426T, L427M, T429M, and V440I. In some embodiments, the mutation is or comprises a modification in a codon of KIR3DL1 encoding an amino acid selected from a group consisting of: R292, F297, I426, L427, and T429. In some embodiments, the mutation in KIR3DL1 is selected from a group consisting of: R292T, F297L, I426T, L427M, and T429M. In some embodiments, the mutation is or comprises a modification in a codon of KIR3DL1 encoding the amino acid R292 and/or F297 in the extracellular domain. In some embodiments, the mutation in the extracellular domain of KIR3DL1 is or comprises the R292T and/or the F297L. In some embodiments, the mutation is or comprises a modification in a codon of KIR3DL1 encoding the amino acid within or near the ITIM 2 of the cytoplasmic domain selected from a group consisting of: I426, L427, and T429. In some embodiments, the mutation within or near the ITIM 2 of the cytoplasmic domain of KIR3DL1 is selected from a group consisting of: I426T, L427M, and T429M. In specific embodiments, the cancer is hematological (or hematogenous) cancer (e.g., leukemia, lymphoma, myeloproliferative neoplasm (MPN), or myelodysplastic syndrome (MDS)) or a solid tumor.

Provided herein are methods of treating a cancer in a subject in need thereof, said method comprising administering a therapeutically effective amount of an FTI to said subject, wherein the cancer is a cancer known to have or determined to have a mutation in KIR3DL2, such as two, three, four, or more mutations, in KIR3DL2. In some embodiments, the methods provided herein include determining the presence of the mutation in KIR3DL2 (e.g., determining the presence of the two, three, four, or more mutations, in KIR3DL2) in a sample from a subject having cancer, and administering a therapeutically effective amount of an FTI to said subject if the mutation in KIR3DL2 is present (e.g., if the two, three, four, or more mutations, in KIR3DL2 are present). In some embodiments, the mutation is or comprises a modification in a codon of KIR3DL2 encoding an amino acid in the extracellular domain, in the cytoplasmic domain, or combinations thereof. In some embodiments, the mutation is or comprises a modification in a codon of KIR3DL2 encoding an amino acid selected from a group consisting of: P319, W323, P324, S333, C336, V341, and Q386. In some embodiments, the mutation in KIR3DL2 is selected from a group consisting of: P319S, W323S, P324S, S333T, C336R, V341I, and Q386E. In some embodiments, the mutation is or comprises a modification in a codon of KIR3DL2 encoding the amino acid C336 and/or Q386. In some embodiments, the mutation in KIR3DL2 is or comprises the C336R and/or the Q386E. In some embodiments, the mutation is or comprises a modification in a codon of KIR3DL2 encoding the extracellular domain amino acid C336. In some embodiments, the mutation in the extracellular domain of KIR3DL2 is C336R. In some embodiments, the mutation is or comprises a modification in a codon of KIR3DL2 encoding the cytoplasmic domain amino acid Q386. In some embodiments, the mutation in the cytoplasmic domain of KIR3DL2 is Q386E. In specific embodiments, the cancer is hematological (or hematogenous) cancer (e.g., leukemia, lymphoma, myeloproliferative neoplasm (MPN), or myelodysplastic syndrome (MDS)) or a solid tumor. In some embodiments, the method further provides determining the VAF of a KIR3DL2 mutation from the sample from the cancer subject. In some embodiments, the method further provides determining the VAF of the KIR3DL2 mutation selected from the group consisting of: a KIR3DL2 C336R mutation, a KIR3DL2 Q386E mutation, or a KIR3DL2 C336R/Q386E mutation, from the sample from the cancer subject, wherein the cancer is AITL. In some embodiments, the AITL is relapsed or refractory AITL. In some embodiments, the AITL is refractory and resistant to a prior standard of care (SOC) treatment selected from the group consisting of: Nivolumab, BEAM/ASCT, DICE, CHOP-E, Brentuximab ved., CEOP, and GemDOX. In some embodiments, the refractory and resistant AITL has a KIR3DL2 Q386E mutation VAF of greater than 5%, 6%, 7%, 8%, or 9%. In some embodiments, the refractory and resistant AITL has a KIR3DL2 Q386E mutation VAF of greater than 5%. In some embodiments, the subject has an improved overall response rate to tipifarnib administration relative to the overall response rate of the prior SOC treatment. In specific embodiments, the VAF is determined by NGS.

Provided herein are methods of treating a cancer in a subject in need thereof, said method comprising administering a therapeutically effective amount of an FTI to said subject, wherein the cancer is a cancer known to have or determined to have a mutation in two, three, four, or each of the, members of the KIR family selected from the group consisting of: KIR2DL1, KIR2DL3, KIR2DL4, KIR3DL1, and KIR3DL2. In some embodiments, the methods provided herein include determining the presence of mutations in two, three, four, or each of the, members of the KIR family selected from the group consisting of: KIR2DL1, KIR2DL3, KIR2DL4, KIR3DL1, and KIR3DL2, in a sample from a subject having cancer, and administering a therapeutically effective amount of an FTI to said subject if the mutations in two, three, four, or each of the, members of the KIR family selected from the group consisting of: KIR2DL1, KIR2DL3, KIR2DL4, KIR3DL1, and KIR3DL2, are present. In some embodiments, the cancer is known to have or determined to have one, two, three, or more, mutations in KIR2DL3 and KIR3DL2. In some embodiments, the cancer is known to have or determined to have one, two, three, or more, mutations in KIR2DL3 and KIR3DL2, wherein the mutation(s) is or comprises a modification in a codon of KIR2DL3 encoding the amino acid R162 and/or E295, and wherein the mutation(s) is or comprises a modification in a codon of KIR3DL2 encoding the amino acid C336 and/or Q386. In some embodiments, the cancer is known to have or determined to have one, two, three, or more, mutations in KIR2DL3 and KIR3DL2, wherein the mutation(s) in the KIR2DL3 is or comprises R162T and/or E295D, and wherein the mutation(s) in the KIR3DL2 is or comprises C336R and/or Q386E. In specific embodiments, the cancer is hematological (or hematogenous) cancer (e.g., leukemia, lymphoma, myeloproliferative neoplasm (MPN), or myelodysplastic syndrome (MDS)) or a solid tumor.

In some embodiments, the methods provided herein for treating cancer in a subject include (a) KIR typing the subject, wherein the subject is a carrier of a mutant KIR family member selected from the group consisting of: KIR2DL1, KIR2DL3, KIR2DL4, KIR3DL1, and/or KIR3DL2, and (b) administering a therapeutically effective amount of an FTI to the subject. In some embodiments, the methods provided herein for selecting a cancer patient for an FTI treatment include (a) KIR typing the subject, wherein the subject is a carrier of a mutant KIR family member selected from the group consisting of: KIR2DL1, KIR2DL3, KIR2DL4, KIR3DL1, and/or KIR3DL2, and (b) administering a therapeutically effective amount of an FTI to the subject. In some embodiments, the subject is a carrier of mutant KIR2DL1, KIR2DL3, KIR2DL4, KIR3DL1, and/or KIR3DL2. In some embodiments, the subject is a carrier of mutant KIR2DL1. In some embodiments, the subject is a carrier of mutant KIR2DL3. In some embodiments, the subject is a carrier of mutant KIR2DL4. In some embodiments, the subject is a carrier of mutant KIR3DL1. In some embodiments, the subject is a carrier of mutant KIR3DL2. In some embodiments, the subject is a carrier of In some embodiments, the subject is a carrier of mutant KIR2DL3 and mutant KIR3DL2.

In some embodiments, the KIR typing of a subject includes determining the presence of a mutant KIR gene in a sample from the subject. In some embodiments, the sample is a blood sample. In some embodiments, the sample is a bone marrow sample. In some embodiments, the sample is peripheral blood mononuclear cells (PBMC). In some embodiments, the sample is enriched natural killer (NK) cells. In some embodiments, the KIR tying is performed by sequencing, Next Generation Sequencing (NGS), Polymerase Chain Reaction (PCR), DNA microarray, Mass Spectrometry (MS), Single Nucleotide Polymorphism (SNP) assay, Immunoblotting assay, or Enzyme-Linked Immunosorbent Assay (ELISA). In one embodiment, the KIR typing is performed by PCR. In one embodiment, the KIR tying is performed by DNA microarray. In one embodiment, the KIR typing is performed by an immunoblotting assay or ELISA.

In some embodiments, the methods provided herein comprise a step of detecting the presence of a mutation in a member of the KIR family in a sample from the subject (e.g., prior to treatment). In some embodiments, the sample from the subject is a bone marrow sample. In some embodiments, the sample from the subject is a blood sample. In some embodiments, the sample from the subject comprises a cell or tissue of the cancer. In some embodiments, the sample is a tumor biopsy. In some embodiments, the cancer is determined to have a mutation in a member of the KIR family. In some embodiments, the mutation is detected by a method selected from the group consisting of sequencing, Next Generation Sequencing (NGS), Polymerase Chain Reaction (PCR), DNA microarray, Mass Spectrometry (MS), Single Nucleotide Polymorphism (SNP) assay, denaturing high-performance liquid chromatography (DHPLC), and Restriction Fragment Length Polymorphism (RFLP) assay. In some embodiment, the methods provided herein comprise treating the subject if the subject is determined to have a mutation in a member of the KIR family (e.g, KIR2DL1, KIR2DL3, KIR2DL4, KIR3DL1, and/or KIR3DL2).

In some embodiments, the method further includes determining a KIR-mutant cancer variant allele frequency (VAF) in a sample from the cancer subject, wherein the KIR-mutant cancer is selected from the group consisting of: a KIR2DL1-mutant, a KIR2DL3-mutant, a KIR2DL4-mutant, a KIR3DL1-mutant, and/or a KIR3DL2-mutant. In some embodiments, the methods provided herein comprise a step of determining the VAF of a mutation in a member of the KIR family (e.g., KIR2DL1, KIR2DL3, KIR2DL4, KIR3DL1, and/or KIR3DL2) in a sample from the cancer subject (e.g., prior to treatment). In some embodiments, the methods provided herein comprise a step of determining the VAF of a KIR3DL2 C336R mutation. In some embodiments, the methods provided herein comprise a step of determining the VAF of a KIR3DL2 Q386E mutation. In some embodiments, the methods provided herein comprise a step of determining the VAF of a KIR3DL2 C336R/Q386E mutation. In some embodiments, the VAF of the mutation is determined by sequencing, such as by Next Generation Sequencing (NGS). In some embodiments, the sample from the subject is a bone marrow sample. In some embodiments, the sample from the subject is a blood sample. In some embodiments, the sample from the subject comprises a cell or tissue of the cancer. In some embodiments, the sample is a tumor biopsy. In some embodiments, the subject is a cancer patient. In some embodiments, the subject has a hematological cancer. In specific embodiments, the subject has AITL. In specific embodiments, the AITL is relapsed or refractory AITL. In some embodiments, the subject is determined to have a VAF of the KIR3DL2 C336R mutation greater than a reference level indicating the subject is likely to be responsive to an FTI treatment. In some embodiments, the subject is determined to have a VAF of the KIR3DL2 Q386E mutation greater than a reference level indicating the subject is likely to be responsive to an FTI treatment. In some embodiments, the subject is determined to have a VAF of the KIR3DL2 C336R mutation greater than a reference level and a VAF of the KIR3DL2 Q386E mutation greater than a reference level indicating the subject is likely to be responsive to an FTI treatment. In specific embodiments, the sample from the subject has a KIR3DL2 C336R mutation VAF of greater than 10%, greater than 15%, or greater than 20%. In specific embodiments, the sample from the subject has a KIR3DL2 Q386E mutation VAF of greater than 5%, greater than 6%, greater than 7%, greater than 8%, or greater than 9%. In specific embodiments, the KIR3DL2 C336R mutation VAF of a subject is greater than 10%. In specific embodiments, the KIR3DL2 C336R mutation VAF of a subject is greater than 15%. In specific embodiments, the KIR3DL2 C336R mutation VAF of a subject is greater than 20%. In specific embodiments, the KIR3DL2 Q386E mutation VAF of a subject is greater than 6%. In specific embodiments, the KIR3DL2 Q386E mutation VAF of a subject is greater than 7%. In specific embodiments, the KIR3DL2 Q386E mutation VAF of a subject is greater than 8%. In specific embodiments, the KIR3DL2 Q386E mutation VAF of a subject is greater than 9%. In some embodiments, the AITL is refractory and resistant to a prior standard of care (SOC) treatment selected from the group consisting of: Nivolumab, BEAM/ASCT, DICE, CHOP-E, Brentuximab ved., CEOP, and GemDOX. In some embodiments, the refractory and resistant AITL has a KIR3DL2 Q386E mutation VAF of greater than 5%, 6%, 7%, 8%, or 9%. In some embodiments, the refractory and resistant AITL has a KIR3DL2 Q386E mutation VAF of greater than 5%. In some embodiments, the subject has an improved overall response rate to tipifarnib administration relative to the overall response rate of the prior SOC treatment.

In some embodiments, the subject is a cancer patient. In some embodiments, the subject has a hematological cancer. In some embodiments, the subject has a solid tumor. The solid tumor can be a benign tumor or a cancer. In some embodiments, the subject has a premalignant condition. The hematological cancer can be lymphoma, T-cell lymphoma, PTCL, AITL, CTCL, relapsed or refractory PTCL, PTCL-NOS, relapsed or refractory AITL, AITL-NOS, ALCL-ALK positive, ALCL-ALK negative, enteropathy-associated T-cell lymphoma, NK lymphoma, extranodal natural killer cell (NK) T-cell lymphoma—nasal type, hepatosplenic T-cell lymphoma, subcutaneous panniculitis-like T-cell lymphoma, EBV associated lymphoma, leukemia, NK leukemia, AML, T-ALL, CML, MDS, MPN, CMML, or JMML. In some embodiments, the patient is a MDS patient. The MDS patient can have very low risk MDS, low risk MDS, intermediate risk MDS, or high risk MDS. In some embodiments, the patient is a lower risk MDS patient, which can have a very low risk MDS, low risk MDS, intermediate risk MDS. In some embodiments, the cancer is HPV negative. In some embodiments, the cancer is hepatocelluar carcinoma, head and neck cancer, salivary gland tumor, thyroid tumor, urothelial cancer, breast cancer, melanoma, gastric cancer, pancreatic cancer, or lung cancer. In some embodiments, the cancer is head and neck squamous cell carcinoma (HNSCC). In some embodiments, the cancer is salivary gland tumor. In some embodiments, the cancer is a thyroid tumor.

In some embodiments, the methods provided herein comprise treating KIR-mutant cancer by administering an FTI to a subject for at least or more than 3 months, 4 months, 5 months, 6 months, 7 months, 8 months, 9 months or 1 year. In some embodiments, an FTI is administered on days 1-21 of a 28-day treatment cycle. In some embodiment, an FTI is administered on days 1-7 of a 28-day treatment cycle. In some embodiments, an FTI is administered on days 1-7 and 15-21 of a 28-day treatment cycle. In some embodiments, an FTI is administered for at least 3 cycles or at least 6 cycles. In some embodiments, an FTI is administered twice a day. In some embodiments, the subject is or remains responsive to treatment with an FTI for at least or more than 3 months, 4 months, 5 months, 6 months, 7 months, 8 months, 9 months or 1 year. In some embodiments, an FTI is tipifarnib. In some embodiments, an FTI (e.g., tipifarnib) is administered at a dose in the range of 200-1200 mg (e.g., orally, twice a day). In some embodiments, an FTI (e.g., tipifarnib) is administered at a dose of 900 mg twice a day (e.g., orally). In some embodiments, an FTI (e.g., tipifarnib) is administered at a dose of 600 mg twice a day (e.g., orally). In some embodiments, an FTI (e.g., tipifarnib) is administered at a dose of 400 mg twice a day (e.g., orally). In some embodiments, an FTI (e.g., tipifarnib) is administered at a dose of 300 mg twice a day (e.g., orally). In some embodiments, an FTI (e.g., tipifarnib) is administered at a dose of 200 mg twice a day (e.g., orally).

In some embodiments, the FTI is selected from the group consisting of tipifarnib, lonafarnib, CP-609,754, BMS-214662, L778123, L744823, L739749, R208176, AZD3409 and FTI-277. In some embodiments, the FTI is administered at a dose of 1-1000 mg/kg body weight. In some embodiments, the FTI is tipifarnib. In some embodiments, an FTI is administered at a dose of 200-1200 mg twice a day (“b.i.d.”). In some embodiments, an FTI is administered at a dose of 200 mg twice a day. In some embodiments, an FTI is administered at a dose of 300 mg twice a day. In some embodiments, an FTI is administered at a dose of 600 mg twice a day. In some embodiments, an FTI is administered at a dose of 900 mg twice a day. In some embodiments, an FTI is administered at a dose of 1200 mg twice a day. In some embodiments, an FTI is administered at a dose of 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, or 1200 mg twice a day. In some embodiments, an FTI is administered daily for a period of one to seven days. In some embodiments, an FTI is administered in alternate weeks. In some embodiments, an FTI is administered on days 1-7 and 15-21 of a 28-day treatment cycle. In some embodiments, the treatment period can continue for up to 12 months. In some embodiments, tipifarnib is administered orally at a dose of 300 mg twice a day on days 1-7 and 15-21 of a 28-day treatment cycle. In some embodiments, tipifarnib is administered orally at a dose of 600 mg twice a day on days 1-7 and 15-21 of a 28-day treatment cycle. In some embodiments, tipifarnib is administered orally at a dose of 900 mg twice a day on days 1-7 and 15-21 of a 28-day treatment cycle.

In some embodiments, the methods provided herein further comprise administering a second active agent or a support care therapy (e.g, a therapeutically effective amount of a second active agent). In some embodiments, an FTI is administered before, during, or after irradiation. In some embodiments, the methods provided herein also include administering a therapeutically effective amount of a secondary active agent or a support care therapy to the subject. In some embodiments, the secondary active agent is a DNA-hypomethylating agent, a therapeutic antibody that specifically binds to a cancer antigen, a hematopoietic growth factor, cytokine, anti-cancer agent, antibiotic, cox-2 inhibitor, immunomodulatory agent, anti-thymocyte globulin, immunosuppressive agent, corticosteroid or a pharmacologically derivative thereof. In some embodiments, the secondary active agent is a DNA-hypomethylating agent, such as azacitidine or decitabine.

In some embodiments, the FTI for use in the compositions and methods provided herein is tipifarnib.

### BRIEF DESCRIPTION OF THE DRAWINGS

FIG. 1. Graph listing mutations in KIR2DL1 determined to be present in samples obtained from patients with PTCL, PTCL-NOS, or AITL, and the resulting response of said patients to treatment with tipifarnib.

FIG. 2. Graph listing mutations in KIR2DL3 determined to be present in samples obtained from patients with PTCL, PTCL-NOS, or AITL, and the resulting response of said patients to treatment with tipifarnib.

FIG. 3. Graph listing mutations in KIR2DL4 determined to be present in samples obtained from patients with PTCL, PTCL-NOS, or AITL, and the resulting response of said patients to treatment with tipifarnib.

FIG. 4. Graph listing mutations in KIR3DL1 determined to be present in samples obtained from patients with PTCL, PTCL-NOS, or AITL, and the resulting response of said patients to treatment with tipifarnib.

FIG. 5. Graph listing mutations in KIR3DL2 determined to be present in samples obtained from patients with PTCL, PTCL-NOS, or AITL, and the resulting response of said patients to treatment with tipifarnib.

FIG. 6. Table correlating mutations in KIR2DL3 (R162T and E295D) and mutations in KIR3DL2 (C336R and Q386E) determined to be present in samples obtained from patients with PTCL, PTCL-NOS, or AITL, and the resulting response of said patients to treatment with tipifarnib.

FIG. 7. Graph listing mutations in KIR3DL2 determined to be present in samples obtained from patients with AITL and the resulting response of said patients to treatment with tipifarnib.

FIG. 8. Graph correlating VAF of specific KIR3DL2 mutations (C336R and/or Q386E), determined to be present in samples obtained from patients with AITL, and the resulting response of said patients to treatment with tipifarnib.

FIG. 9. Chart correlating VAF of KIR3DL2 Q386E mutation, determined to be present in samples obtained from patients with AITL, and the resulting response of said patients to treatment with tipifarnib, relative to response rates resulting from prior SOC treatments.

### DETAILED DESCRIPTION

Provided herein are methods for population selection of cancer patients for treatment with a farnesyltransferase inhibitor (FTI). The methods provided herein are based, in part, on the discovery that Killer Cell Immunoglobulin-Like Receptor (KIR) mutation status can be used to predict responsiveness of a cancer patient to an FTI treatment. KIR molecules are transmembrane glycoproteins expressed by natural killer cells and certain subsets of T cells.

In some embodiments, the methods provided herein include (a) determining the presence of a mutation in a member of the KIR family in a sample from the subject, and subsequently (b) administering a therapeutically effective amount of an FTI (e.g., tipifarnib) to the subject if the sample is determined to have a mutation in a member of the KIR family. In some embodiments, the methods provided herein include (a) determining the presence of a mutation in KIR2DL1, KIR2DL3, KIR2DL4, KIR3DL1, and/or KIR3DL2 in a sample from the subject, and subsequently (b) administering a therapeutically effective amount of an FTI (e.g., tipifarnib) to the subject if the sample is determined to have a mutation in KIR2DL1, KIR2DL3, KIR2DL4, KIR3DL1, and/or KIR3DL2.

In some embodiments, the methods provided herein include (a) determining the presence of a KIR2DL and/or KIR3DL mutation in a sample from the subject, and subsequently (b) administering a therapeutically effective amount of an FTI (e.g., tipifarnib) to the subject if the sample is determined to have a KIR2DL and/or KIR3DL mutation.

In some embodiments, the methods provided herein include determining the presence of a KIR2DL and/or KIR3DL mutation (e.g., a mutation in KIR2DL1, KIR2DL3, KIR2DL4, KIR3DL1, and/or KIR3DL2). In some embodiments, the methods provided herein include (a) determining the presence of a KIR2DL and/or KIR3DL mutation (e.g., a mutation in KIR2DL1, KIR2DL3, KIR2DL4, KIR3DL1, and/or KIR3DL2) in a sample from the subject, and subsequently (b) administering a therapeutically effective amount of an FTI (e.g., tipifarnib) to the subject if the sample is determined to have a KIR2DL and/or KIR3DL mutation (e.g., a mutation in KIR2DL1, KIR2DL3, KIR2DL4, KIR3DL1, and/or KIR3DL2).

In some embodiments, provided herein are methods of treating a cancer in a subject comprising: (a) determining the presence or absence of a mutation in a member of the KIR family in a sample from said subject, and subsequently (b) administering a therapeutically effective amount of an FTI (e.g., tipifarnib) to said subject if said sample is determined to have a mutation in a member of the KIR family. In some embodiments, said sample has a mutation, two or more mutations, or three or more mutations, in KIR2DL1. In some embodiments, said sample has a mutation, two or more mutations, or three or more mutations, in KIR2DL3. In some embodiments, said sample has a mutation, two or more mutations, or three or more mutations, in KIR2DL4. In some embodiments, said sample has a mutation, two or more mutations, or three or more mutations, in KIR3DL1. In some embodiments, said sample has a mutation, two or more mutations, or three or more mutations, in KIR3DL2. In some embodiments, provided herein are methods of treating a cancer in a subject comprising: (a) determining the presence or absence of a mutation in KIR2DL1, KIR2DL3, KIR2DL4, KIR3DL1, and/or KIR3DL2 in a sample from said subject, and subsequently (b) administering a therapeutically effective amount of an FTI (e.g., tipifarnib) to said subject if said sample is determined to have a mutation in KIR2DL1, KIR2DL3, KIR2DL4, KIR3DL1, and/or KIR3DL2.

In some embodiments, provided herein are methods of treating a KIR-mutant cancer in a subject comprising administering a therapeutically effective amount of an FTI (e.g., tipifarnib) to said subject. In some embodiments, provided herein are methods of treating a KIR-mutant cancer in a subject comprising administering a therapeutically effective amount of an FTI (e.g., tipifarnib) to said subject, wherein the KIR-mutant cancer is a cancer known to have or determined to have a mutation in one or more genes or proteins of the KIR family (e.g., wherein cells of the cancer have a mutation in a gene of the KIR family). The member of the KIR family can be KIR2DL1, KIR2DL3, KIR2DL4, KIR3DL1, and/or KIR3DL2. In some embodiments, the KIR-mutant cancer has an amino acid modification at a codon of KIR2DL1 selected from a group consisting of M65, H77, A83, S88, T91, L140, N178, G179, D184, R197, F202, and H203 (or any combination thereof). In some embodiments, the KIR-mutant cancer has an amino acid modification at a codon of KIR2DL3 selected from a group consisting of F66, R162, R169, F171, S172, E295, R318, I330, I331, and V332 (or any combination thereof). In some embodiments, the KIR-mutant cancer has an amino acid modification at a codon of KIR2DL4 selected from a group consisting of R50, H52, R55, N58, T61, K65, Q149, I154, E162, L166, I174, A238, and S267 (or any combination thereof). In some embodiments, the KIR-mutant cancer has an amino acid modification at a codon of KIR3DL1 selected from a group consisting of R292, F297, P336, R409, R413, I426, L427, T429, and V440 (or any combination thereof). In some embodiments, the KIR-mutant cancer has an amino acid modification at a codon of KIR3DL2 selected from a group consisting of P319, W323, P324, S333, C336, V341, and Q386 (or any combination thereof). In some embodiments, the KIR-mutant cancer has a mutation in amino acid R162 and/or E295 of KIR2DL3, and/or a mutation in amino acid C336 and/or Q386 of KIR3DL2. In some embodiments, the KIR-mutant cancer has a mutation in an amino acid modification at a codon (or two, three, four, or more, mutations, in two, three, four, or more, amino acid modifications at two, three, four, or more codons, respectively) selected from the group consisting of: (1) KIR2DL1 selected from a group consisting of M65, H77, A83, S88, T91, L140, N178, G179, D184, R197, F202, and H203 (or any combination thereof); (2) KIR2DL3 selected from a group consisting of F66, R162, R169, F171, S172, E295, R318, I330, I331, and V332 (or any combination thereof); (3) KIR2DL4 selected from a group consisting of R50, H52, R55, N58, T61, K65, Q149, I154, E162, L166, I174, A238, and S267 (or any combination thereof); (4) KIR3DL1 selected from a group consisting of R292, F297, P336, R409, R413, I426, L427, T429, and V440 (or any combination thereof); and (5) KIR3DL2 selected from a group consisting of P319, W323, P324, S333, C336, V341, and Q386 (or any combination thereof). In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, provided herein are methods of treating a cancer in a subject in need thereof comprising selectively administering a therapeutically effective amount of an FTI (e.g., tipifarnib) to a subject having a mutation in one or more genes of the KIR family (such as KIR2DL1, KIR2DL3, KIR2DL4, KIR3DL1, and/or KIR3DL2). In some embodiments, the KIR-mutant cancer has an amino acid modification at a codon of KIR2DL1 selected from a group consisting of M65, H77, A83, S88, T91, L140, N178, G179, D184, R197, F202, and H203 (or any combination thereof). In some embodiments, the KIR-mutant cancer has an amino acid modification at a codon of KIR2DL3 selected from a group consisting of F66, R162, R169, F171, S172, E295, R318, I330, I331, and V332 (or any combination thereof). In some embodiments, the KIR-mutant cancer has an amino acid modification at a codon of KIR2DL4 selected from a group consisting of R50, H52, R55, N58, T61, K65, Q149, I154, E162, L166, 1174, A238, and S267 (or any combination thereof). In some embodiments, the KIR-mutant cancer has an amino acid modification at a codon of KIR3DL1 selected from a group consisting of R292, F297, P336, R409, R413, I426, L427, T429, and V440 (or any combination thereof). In some embodiments, the KIR-mutant cancer has an amino acid modification at a codon of KIR3DL2 selected from a group consisting of P319, W323, P324, S333, C336, V341, and Q386 (or any combination thereof). In some embodiments, the KIR-mutant cancer has a mutation in amino acid R162 and/or E295 of KIR2DL3, and/or a mutation in amino acid C336 and/or Q386 of KIR3DL2. In some embodiments, the KIR-mutant cancer has a mutation in an amino acid modification at a codon (or two, three, four, or more, mutations, in two, three, four, or more, amino acid modifications at two, three, four, or more codons, respectively) selected from the group consisting of: (1) KIR2DL1 selected from a group consisting of M65, H77, A83, S88, T91, L140, N178, G179, D184, R197, F202, and H203 (or any combination thereof); (2) KIR2DL3 selected from a group consisting of F66, R162, R169, F171, S172, E295, R318, I330, I331, and V332 (or any combination thereof); (3) KIR2DL4 selected from a group consisting of R50, H52, R55, N58, T61, K65, Q149, I154, E162, L166, I174, A238, and S267 (or any combination thereof); (4) KIR3DL1 selected from a group consisting of R292, F297, P336, R409, R413, I426, L427, T429, and V440 (or any combination thereof); and (5) KIR3DL2 selected from a group consisting of P319, W323, P324, S333, C336, V341, and Q386 (or any combination thereof). In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, provided herein are methods of treating a cancer in a subject comprising: (a) obtaining a tissue or plasma sample from a subject (e.g., a sample containing cancer cells such as tumor biopsy); (b) detecting the presence of a mutation in one or more members of the KIR family in the sample; (c) administering a therapeutically effective amount of an FTI (e.g., tipifarnib) to the subject determined to have a mutation in a member of the KIR family. The member of the KIR family can be KIR2DL1, KIR2DL3, KIR2DL4, KIR3DL1, and/or KIR3DL2. In some embodiments, the KIR-mutant cancer has an amino acid modification at a codon of KIR2DL1 selected from a group consisting of M65, H77, A83, S88, T91, L140, N178, G179, D184, R197, F202, and H203 (or any combination thereof). In some embodiments, the KIR-mutant cancer has an amino acid modification at a codon of KIR2DL3 selected from a group consisting of F66, R162, R169, F171, S172, E295, R318, I330, I331, and V332 (or any combination thereof). In some embodiments, the KIR-mutant cancer has an amino acid modification at a codon of KIR2DL4 selected from a group consisting of R50, H52, R55, N58, T61, K65, Q149, I154, E162, L166, I174, A238, and S267 (or any combination thereof). In some embodiments, the KIR-mutant cancer has an amino acid modification at a codon of KIR3DL1 selected from a group consisting of R292, F297, P336, R409, R413, I426, L427, T429, and V440 (or any combination thereof). In some embodiments, the KIR-mutant cancer has an amino acid modification at a codon of KIR3DL2 selected from a group consisting of P319, W323, P324, S333, C336, V341, and Q386 (or any combination thereof). In some embodiments, the KIR-mutant cancer has a mutation in amino acid R162 and/or E295 of KIR2DL3, and/or a mutation in amino acid C336 and/or Q386 of KIR3DL2. In some embodiments, the KIR-mutant cancer has a mutation in an amino acid modification at a codon (or two, three, four, or more, mutations, in two, three, four, or more, amino acid modifications at two, three, four, or more codons, respectively) selected from the group consisting of: (1) KIR2DL1 selected from a group consisting of M65, H77, A83, S88, T91, L140, N178, G179, D184, R197, F202, and H203 (or any combination thereof); (2) KIR2DL3 selected from a group consisting of F66, R162, R169, F171, S172, E295, R318, I330, I331, and V332 (or any combination thereof); (3) KIR2DL4 selected from a group consisting of R50, H52, R55, N58, T61, K65, Q149, I154, E162, L166, I174, A238, and S267 (or any combination thereof); (4) KIR3DL1 selected from a group consisting of R292, F297, P336, R409, R413, I426, L427, T429, and V440 (or any combination thereof); and (5) KIR3DL2 selected from a group consisting of P319, W323, P324, S333, C336, V341, and Q386 (or any combination thereof). In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, provided herein are methods of treating a cancer in a subject having a mutation in one or more members of the KIR family comprising administering an FTI (e.g., tipifarnib) to said subject. In some embodiments, provided herein are methods of treating a cancer in a subject having a cancer and a mutation in one or more members of the KIR family comprising administering a therapeutically effective amount of an FTI (e.g., tipifarnib) to said subject. The member of the KIR family can be KIR2DL1, KIR2DL3, KIR2DL4, KIR3DL1, and/or KIR3DL2. In some embodiments, the KIR-mutant cancer has an amino acid modification at a codon of KIR2DL1 selected from a group consisting of M65, H77, A83, S88, T91, L140, N178, G179, D184, R197, F202, and H203 (or any combination thereof). In some embodiments, the KIR-mutant cancer has an amino acid modification at a codon of KIR2DL3 selected from a group consisting of F66, R162, R169, F171, S172, E295, R318, I330, I331, and V332 (or any combination thereof). In some embodiments, the KIR-mutant cancer has an amino acid modification at a codon of KIR2DL4 selected from a group consisting of R50, H52, R55, N58, T61, K65, Q149, I154, E162, L166, I174, A238, and S267 (or any combination thereof). In some embodiments, the KIR-mutant cancer has an amino acid modification at a codon of KIR3DL1 selected from a group consisting of R292, F297, P336, R409, R413, I426, L427, T429, and V440 (or any combination thereof). In some embodiments, the KIR-mutant cancer has an amino acid modification at a codon of KIR3DL2 selected from a group consisting of P319, W323, P324, S333, C336, V341, and Q386 (or any combination thereof). In some embodiments, the KIR-mutant cancer has a mutation in amino acid R162 and/or E295 of KIR2DL3, and/or a mutation in amino acid C336 and/or Q386 of KIR3DL2. In some embodiments, the KIR-mutant cancer has a mutation in an amino acid modification at a codon (or two, three, four, or more, mutations, in two, three, four, or more, amino acid modifications at two, three, four, or more codons, respectively) selected from the group consisting of: (1) KIR2DL1 selected from a group consisting of M65, H77, A83, S88, T91, L140, N178, G179, D184, R197, F202, and H203 (or any combination thereof); (2) KIR2DL3 selected from a group consisting of F66, R162, R169, F171, S172, E295, R318, I330, I331, and V332 (or any combination thereof); (3) KIR2DL4 selected from a group consisting of R50, H52, R55, N58, T61, K65, Q149, I154, E162, L166, I174, A238, and S267 (or any combination thereof); (4) KIR3DL1 selected from a group consisting of R292, F297, P336, R409, R413, I426, L427, T429, and V440 (or any combination thereof); and (5) KIR3DL2 selected from a group consisting of P319, W323, P324, S333, C336, V341, and Q386 (or any combination thereof). In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

The subject can be a mammal, for example, a human. The subject can be male or female, and can be an adult, child or infant. The subject can be a patient who has cancer (e.g., has been diagnosed with a cancer).

The cancer treated in accordance with the methods provided herein can be any cancer described herein, for example, solid tumors or hematological cancers, such as myeloproliferative neoplasm (MPN), myelodysplastic syndrome (MDS), leukemia, and lymphoma. The hematological cancer treated in accordance with the methods provided herein can be any hematological cancer described herein, for example, lymphoma, T-cell lymphoma, PTCL, AITL, CTCL, relapsed or refractory PTCL, PTCL-NOS, relapsed or refractory AITL, AITL-NOS, ALCL-ALK positive, ALCL-ALK negative, enteropathy-associated T-cell lymphoma, NK lymphoma, extranodal natural killer cell (NK) T-cell lymphoma—nasal type, hepatosplenic T-cell lymphoma, subcutaneous panniculitis-like T-cell lymphoma, EBV associated lymphoma, leukemia, NK leukemia, AML, T-ALL, CML, MDS, MPN, CMML, or JMML. In some embodiments, the subject has a solid tumor. The solid tumor treated in accordance with the methods provided herein can be, for example, a benign tumor or a cancer. The cancer treated in accordance with the methods provided herein can be, for example, hepatocelluar carcinoma, head and neck cancer, salivary gland tumor, thyroid tumor, urothelial cancer, breast cancer, melanoma, gastric cancer, pancreatic cancer, lung cancer, head and neck squamous cell carcinoma (HNSCC), salivary gland tumor, or thyroid tumor.

In some embodiments, the FTI is tipifarnib, arglabin, perrilyl alcohol, SCH-66336, L778123, L739749, FTI-277, L744832, CP-609,754, R208176, AZD3409, and BMS-214662. In some embodiments, the FTI is tipifarnib. It is also contemplated that a pharmaceutically acceptable salt of an FTI can be used in the methods described herein.

### 1. Definitions

As used herein, the articles “a,” “an,” and “the” refer to one or to more than one of the grammatical object of the article. By way of example, a sample refers to one sample or two or more samples.

As used herein, the term “subject” refers to a mammal. A subject can be a human or a non-human mammal such as a dog, cat, bovid, equine, mouse, rat, rabbit, or transgenic species thereof.

As used herein, the term “sample” refers to a material or mixture of materials containing one or more components of interest. A sample from a subject refers to a sample obtained from the subject, including samples of biological tissue or fluid origin, obtained, reached, or collected in vivo or in situ. A sample can be obtained from a region of a subject containing precancerous or cancer cells or tissues. Such samples can be, but are not limited to, organs, tissues, fractions and cells isolated from a mammal. Exemplary samples include lymph node, whole blood, partially purified blood, serum, bone marrow, and peripheral blood mononuclear cells (“PBMC”). A sample also can be a tissue biopsy. Exemplary samples also include cell lysate, a cell culture, a cell line, a tissue, oral tissue, gastrointestinal tissue, an organ, an organelle, a biological fluid, a blood sample, a urine sample, a skin sample, and the like.

As used herein, the term “cancer” or “cancerous” refers to the physiological condition in mammals that is typically characterized by unregulated cell growth. Examples of cancer include, but are not limited to, hematological cancers (e.g., multiple myeloma, lymphoma and leukemia), and solid tumors. The cancer can be related to Human papillomavirus (HPV+ or HPV positive), or unrelated to HPV (HPV− or HPV negative). As used herein, the term “premalignant condition” refers to a condition associated with an increased risk of cancer, which, if left untreated, can lead to cancer. A premalignant condition can also refer to non-invasive cancer that have not progressed into aggressive, invasive stage. Examples of premalignant conditions include, but are not limited to, actinic cheilitis, Barrett's esophagus, atrophic gastritis, ductal carcinoma in situ, Dyskeratosis congenita, Sideropenic dysphagia, Lichen planus, Oral submucous fibrosis, Solar elastosis, cervical dysplasia, polyps, leukoplakia, erythroplakia, squamous intraepithelial lesion, a pre-malignant disorder, and a pre-malignant immunoproliferative disorder.

As used herein, the term “hematologic cancer” refers to a cancer of the blood or bone marrow. Examples of hematological (or hematogenous) cancers include, but are not limited to, myeloproliferative neoplasm (MPN), myelodysplastic syndrome (MDS), leukemia, and lymphoma. Further examples of hematological (or hematogenous) cancers include, but are not limited to, acute leukemias (such as acute lymphocytic leukemia (ALL), T-cell acute lymphocytic leukemia (T-ALL), acute myelocytic leukemia (AML), acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia (sometimes referred to as chronic myeloid leukemia) (CML), and chronic lymphocytic leukemia (CLL)), chronic myelomonocytic leukemia (CMML), juvenile myelomonocytic leukemia (JMML), polycythemia vera, natural killer cell lymphoma (NK lymphoma), natural killer cell leukemia (NK leukemia), Hodgkin's disease, non-Hodgkin's lymphoma (indolent and high grade forms), multiple myeloma, T-cell lymphoma, peripheral T-cell lymphomas (PTCL), PTCL not otherwise specified (PTCL-NOS), relapsed or refractory PTCL, angioimmunoblastic T-cell lymphoma (AITL), AITL not otherwise specified (AITL-NOS), relapsed or refractory AITL, cutaneous T-Cell lymphoma (CTCL), anaplastic large cell lymphoma (ALCL)-anaplastic lymphoma kinase (ALK) positive, anaplastic large cell lymphoma (ALCL)-anaplastic lymphoma kinase (ALK) negative, enteropathy-associated T-cell lymphoma, extranodal natural killer (NK) T-cell lymphoma, nasal type, hepatosplenic T-cell lymphoma, subcutaneous panniculitis-like T-cell lymphoma, Waldenstrom's macroglobulinemia, heavy chain disease, agnogenic myeloid metaplasia, familial erythrophagocytic lymphohistiocytosis, hairy cell leukemia and myelodysplasia.

As used herein, the term “solid tumor” or “solid tumors” refers to abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors include, but are not limited to, sarcomas and carcinomas, including head and neck carcinoma (head and neck cancers), head and neck squamous cell carcinoma (HNSCC), salivary cancers, salivary gland cancers, fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, chronic granulomatous disease, cancers of the upper digestive tract, gastric cancer, colon carcinoma (colon cancer), lymphoid malignancy, carcinoma of the pancreas (pancreatic cancer), breast carcinoma (breast cancer), lung cancers, melanoma, malignant melanoma, non-small-cell lung carcinoma (NSCLC), ovarian cancer, prostate cancer, urothelial cancers, hepatocellular carcinoma, squamous cell carcinoma, basal cell carcinoma, adenocarcinoma, adrenal carcinoma, sweat gland carcinoma, thyroid carcinoma (thyroid cancer), transitional cell carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma (renal cell cancer), hepatoma, bile duct carcinoma, choriocarcinoma, Wilms' tumor, cervical cancer, testicular tumor, seminoma, bladder carcinoma (bladder cancer), and brain cancer or CNS tumors (such as a glioma (e.g., brainstem glioma and mixed gliomas), glioblastoma (also known as glioblastoma multiforme) astrocytoma, CNS lymphoma, germinoma, meduloblastoma, Schwannoma craniopharyogioma, ependymoma, pineaioma, hemangioblastoma, acoustic neuroma, oligodendroglioma, menangioma, neuroblastoma, retinoblastoma and brain metastases).

Leukemia refers to malignant neoplasms of the blood-forming tissues. Various forms of leukemias are described, for example, in U.S. Pat. No. 7,393,862 and U.S. provisional patent application No. 60/380,842, filed May 17, 2002, the entireties of which are incorporated herein by reference. Although viruses reportedly cause several forms of leukemia in animals, causes of leukemia in humans are to a large extent unknown. The Merck Manual, 944-952 (17th ed. 1999). Transformation to malignancy typically occurs in a single cell through two or more steps with subsequent proliferation and clonal expansion. In some leukemias, specific chromosomal translocations have been identified with consistent leukemic cell morphology and special clinical features (e.g., translocations of 9 and 22 in chronic myelocytic leukemia, and of 15 and 17 in acute promyelocytic leukemia). Acute leukemias are predominantly undifferentiated cell populations and chronic leukemias more mature cell forms.

Acute leukemias are divided into lymphoblastic (ALL) and non-lymphoblastic (ANLL) types. The Merck Manual, 946-949 (17^{th} ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease which is established by laboratory findings and bone marrow examination. ANLL, also known as acute myelogenous leukemia or AML, occurs at all ages and is the more common acute leukemia among adults; it is the form usually associated with irradiation as a causative agent. In some embodiments, provided herein are methods for treating a AML patient with an FTI, or methods for selecting patients for FTI treatment.

Standard procedures treat AML patients usually include 2 chemotherapy (chemo) phases: remission induction (or induction) and consolidation (post-remission therapy). The first part of treatment (remission induction) is aimed at getting rid of as many leukemia cells as possible. The intensity of the treatment can depend on a person's age and health. Intensive chemotherapy is often given to people under the age of 60. Some older patients in good health can benefit from similar or slightly less intensive treatment. People who are much older or are in poor health are not suitable for intensive chemotherapies.

In younger patients, such as those under 60, induction often involves treatment with 2 chemo drugs, cytarabine (ara-C) and an anthracycline drug such as daunorubicin (daunomycin) or idarubicin. Sometimes a third drug, cladribine (Leustatin, 2-CdA), is given as well. The chemo is usually given in the hospital and lasts about a week. In rare cases where the leukemia has spread to the brain or spinal cord, chemo may also be given into the cerebrospinal fluid (CSF). Radiation therapy might be used as well.

Induction is considered successful if remission is achieved. However, the AML in some patients can be refractory to induction. In patients who respond to induction, further treatment is then given to try to destroy remaining leukemia cells and help prevent a relapse, which is called consolidation. For younger patients, the main options for consolidation therapy are: several cycles of high-dose cytarabine (ara-C) chemo (sometimes known as HiDAC); allogeneic (donor) stem cell transplant; and autologous stem cell transplant.

Chronic leukemias are described as being lymphocytic (CLL) or myelocytic (CML). The Merck Manual, 949-952 (17^{th} ed. 1999). CLL is characterized by the appearance of mature lymphocytes in blood, bone marrow, and lymphoid organs. The hallmark of CLL is sustained, absolute lymphocytosis (>5,000/μL) and an increase of lymphocytes in the bone marrow. Most CLL patients also have clonal expansion of lymphocytes with B-cell characteristics. CLL is a disease of middle or old age. In CML, the characteristic feature is the predominance of granulocytic cells of all stages of differentiation in blood, bone marrow, liver, spleen, and other organs. In the symptomatic patient at diagnosis, the total white blood cell (WBC) count is usually about 200,000/μL, but may reach 1,000,000/μL. CML is relatively easy to diagnose because of the presence of the Philadelphia chromosome. Bone marrow stromal cells are well known to support CLL disease progression and resistance to chemotherapy. Disrupting the interactions between CLL cells and stromal cells is an additional target of CLL chemotherapy.

Additionally, other forms of CLL include prolymphocytic leukemia (PLL), Large granular lymphocyte (LGL) leukemia, Hairy cell leukemia (HCL). The cancer cells in PLL are similar to normal cells called prolymphocytes—immature forms of B lymphocytes (B-PLL) or T lymphocytes (T-PLL). Both B-PLL and T-PLL tend to be more aggressive than the usual type of CLL. The cancer cells of LGL are large and have features of either T cells or NK cells. Most LGL leukemias are slow-growing, but a small number are more aggressive. HCL is another cancer of lymphocytes that tends to progress slowly, and accounts for about 2% of all leukemias. The cancer cells are a type of B lymphocyte but are different from those seen in CLL.

Chronic myelomonocytic leukemia (CMML) is classified as a myelodysplastic/myeloproliferative neoplasm by the 2008 World Health Organization classification of hematopoietic tumors. CMML patients have a high number of monocytes in their blood (at least 1,000 per mm³). Two classes—myelodysplastic and myeloproliferative—have been distinguished upon the level of the white blood cell count (threshold 13 G/L). Often, the monocyte count is much higher, causing their total white blood cell count to become very high as well. Usually there are abnormal cells in the bone marrow, but the amount of blasts is below 20%. About 15% to 30% of CMML patients go on to develop acute myeloid leukemia. The diagnosis of CMML rests on a combination of morphologic, histopathologic and chromosomal abnormalities in the bone marrow. The Mayo prognostic model classified CMML patients into three risk groups based on: increased absolute monocyte count, presence of circulating blasts, hemoglobin <10 gm/dL and platelets <100×10⁹/L. The median survival was 32 months, 18.5 months and 10 months in the low, intermediate, and high-risk groups, respectively. The Groupe Francophone des (GFM) score segregated CMML patients into three risk groups based on: age >65 years, WBC>15×10⁹/L, anemia, platelets <100×10⁹/L, and ASXL1 mutation status. After a median follow-up of 2.5 years, survival ranged from not reached in the low-risk group to 14.4 months in the high-risk group.

Juvenile myelomonocytic leukemia (JMML) is a serious chronic leukemia that affects children mostly aged 4 and under. The average age of patients at diagnosis is 2 years old. The World Health Organization has categorized JMML as a mixed myelodysplastic and myeloproliferative disorder. The JMML encompasses diagnoses formerly referred to as Juvenile Chronic Myeloid Leukemia (JCML), Chronic Myelomonocytic Leukemia of Infancy, and Infantile Monosomy 7 Syndrome.

Lymphoma refers to cancers that originate in the lymphatic system. Lymphoma is characterized by malignant neoplasms of lymphocytes—B lymphocytes (B cell lymphoma), T lymphocytes (T-cell lymphoma), and natural killer cells (NK cell lymphoma). Lymphoma generally starts in lymph nodes or collections of lymphatic tissue in organs including, but not limited to, the stomach or intestines. Lymphoma may involve the marrow and the blood in some cases. Lymphoma may spread from one site to other parts of the body.

The treatments of various forms of lymphomas are described, for example, in U.S. Pat. No. 7,468,363, the entirety of which is incorporated herein by reference. Such lymphomas include, but are not limited to, Hodgkin's lymphoma, non-Hodgkin's lymphoma, cutaneous B-cell lymphoma, activated B-cell lymphoma, Diffuse Large B-Cell Lymphoma (DLBCL), mantle cell lymphoma (MCL), follicular lymphoma (FL; including but not limited to FL grade I, FL grade II), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma (CTCL) and mantle zone lymphoma and low grade follicular lymphoma.

Non-Hodgkin's lymphoma (NHL) is the fifth most common cancer for both men and women in the United States, with an estimated 63,190 new cases and 18,660 deaths in 2007. Jemal A, et al., CA Cancer J Clin 2007; 57(1):43-66. The probability of developing NHL increases with age and the incidence of NHL in the elderly has been steadily increasing in the past decade, causing concern with the aging trend of the U.S. population. Id. Clarke C A, et al., Cancer 2002; 94(7):2015-2023.

DLBCL accounts for approximately one-third of non-Hodgkin's lymphomas. While some DLBCL patients are cured with traditional chemotherapy, the remainders die from the disease. Anticancer drugs cause rapid and persistent depletion of lymphocytes, possibly by direct apoptosis induction in mature T and B cells. See K. Stahnke. et al., Blood 2001, 98:3066-3073. Absolute lymphocyte count (ALC) has been shown to be a prognostic factor in follicular non-Hodgkin's lymphoma and recent results have suggested that ALC at diagnosis is an important prognostic factor in DLBCL.

DLBCL can be divided into distinct molecular subtypes according to their gene profiling patterns: germinal-center B-cell-like DLBCL (GCB-DLBCL), activated B-cell-like DLBCL (ABC-DLBCL), and primary mediastinal B-cell lymphoma (PMBL) or unclassified type. These subtypes are characterized by distinct differences in survival, chemo-responsiveness, and signaling pathway dependence, particularly the NF-κB pathway. See D. Kim et al., Journal of Clinical Oncology, 2007 ASCO Annual Meeting Proceedings Part I. Vol 25, No. 18S (June 20 Supplement), 2007: 8082. See Bea S, et al., Blood 2005; 106: 3183-90; Ngo V. N. et al., Nature 2011; 470: 115-9. Such differences have prompted the search for more effective and subtype-specific treatment strategies in DLBCL. In addition to the acute and chronic categorization, neoplasms are also categorized based upon the cells giving rise to such disorder into precursor or peripheral. See e.g., U.S. patent Publication No. 2008/0051379, the disclosure of which is incorporated herein by reference in its entirety. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, Diffuse large B-cell lymphoma (DLBCL) and Burkitt lymphoma. In over 95 percent of CLL cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype. Notwithstanding these classifications, however, the pathological impairment of normal hematopoiesis is the hallmark of all leukemias.

PTCL consists of a group of rare and usually aggressive (fast-growing) NHLs that develop from mature T-cells. PTCLs collectively account for about 4 to 10 percent of all NHL cases, corresponding to an annual incidence of 2,800-7,200 patients per year in the United States. By some estimates, the incidence of PTCL is growing significantly, and the increasing incidence may be driven by an aging population. PTCLs are sub-classified into various subtypes, including Anaplastic large cell lymphoma (ALCL), ALK positive; ALCL, ALK negative; Angioimmunoblastic T-cell lymphoma (AITL); AITL not otherwise specified (AITL-NOS); Enteropathy-associated T-cell lymphoma; Extranodal natural killer (NK) T-cell lymphoma, nasal type; Hepatosplenic T-cell lymphoma; PTCL not otherwise specified (PTCL-NOS); and Subcutaneous panniculitis-like T-cell lymphoma. Each of these subtypes are typically considered to be separate diseases based on their distinct clinical differences. Most of these subtypes are rare; the three most common subtypes are PTCL NOS, AITL, and ALCL, and these collectively account for approximately 70 percent of all PTCL cases. ALCL can be cutaneous ALCL or systemic ALCL. In some embodiments herein, the PTCL is relapsed or refractory PTCL. In some embodiments, the PTCL is relapsed or refractory advanced PTCL. In some embodiments herein, the AITL is relapsed or refractory AITL. In some embodiments herein, the PTCL is PTCL-NOS. In some embodiments herein, the PTCL is AITL-NOS.

For most PTCL subtypes, the frontline treatment regimen is typically combination chemotherapy, such as CHOP (cyclophosphamide, doxorubicin, vincristine, prednisone), EPOCH (etoposide, vincristine, doxorubicin, cyclophosphamide, prednisone), or other multi-drug regimens. Patients who relapse or are refractory to frontline treatments are typically treated with gemcitabine in combination with other chemotherapies, including vinorelbine (Navelbine®) and doxorubicin (Doxil®) in a regimen called GND, or other chemotherapy regimens such as DHAP (dexamethasone, cytarabine, cisplatin) or ESHAP (etoposide, methylprednisolone, cytarabine, and cisplatin).

Because most patients with PTCL will relapse, some oncologists recommend giving high-dose chemotherapy followed by an autologous stem cell transplant to some patients who had a good response to their initial chemotherapy. Recent, non-cytotoxic therapies that have been approved for relapsed or refractory PTCL, such as pralatrexate (Folotyn®), romidepsin (Istodax®) and belinostat (Beleodaq®), are associated with relatively low objective response rates (25-27% overall response rate, or ORR) and relatively short durations of response (8.2-9.4 months). Accordingly, the treatment of relapsed/refractory PTCL remains a significant unmet medical need.

T cells can be separated into three major groups based on function: cytotoxic T cells, helper T cells (Th), and regulatory T cells (Tregs). Differential expression of markers on the cell surface, as well as their distinct cytokine secretion profiles, provide valuable clues to the diverse nature and function of T cells. For example, CD8+ cytotoxic T cells destroy infected target cells through the release of perforin, granzymes, and granulysin, whereas CD4+ T helper cells have little cytotoxic activity and secrete cytokines that act on other leucocytes such as B cells, macrophages, eosinophils, or neutrophils to clear pathogens. Tregs suppress T-cell function by several mechanisms including binding to effector T-cell subsets and preventing secretion of their cytokines. Helper T cells can be further categorized into difference classes, including e.g., Th1, Th2, Th9, Th17, and Tfh cells. Differentiation of CD4+ T cells into Th1 and Th2 effector cells is largely controlled by the transcription factors TBX21 (T-Box Protein 21; T-bet) and GATA3 (GATA3), respectively. Both TBX21 and GATA3 are transcription factors that are master regulators of gene expression profiles in T helper (Th) cells, skewing Th polarization into Th1 and Th2 differentiation pathways, respectively. Thus, Th1 cells are characterized by high expression levels of TBX21 and the target genes activated by TBX21, and low expression levels of GATA3 and genes activated by GATA3. To the contrary, Th2 cells are characterized by high expression levels of GATA3 and the target genes activated by GATA3, and low expression levels of TBX21 and genes activated by TBX21. PTCL and its subtypes (e.g. PTCL NOS) can be categorized based on Th1 or Th2 lineage derivation.

AITL is characterized histologically by a tumor cell component and a non-tumor cell component. The tumor cell component comprises polymorphous medium-sized neoplastic cells derived from an unique T-cell subset located in lymph nodes germinal centers called follicular helper T cells (TFH). TFH express CXCL13, VEGF and angpt1. CXCL13 can induce the expression of CXCL12 in mesenchymal cells. VEGF and angiopoietin induce the formation of venules of endothelial cells that express CXCL12. The non-tumor cell component comprises prominent arborizing blood vessels, proliferation of follicular dendritic cells, and scattered EBV+ B-cell blasts. Visualization of arborizing blood vessels is a hallmark of the diagnosis of AITL. By visualizing the vessels (endothelial venules), CXCL12 expressing endothelial cells can be identified. Targeted loss of CXCL12 expression in vascular endothelial cells translates to loss of T cell tumors in lymph nodes, spleen and bone marrow (Pitt et al., 2015, “CXCL12-Producing Vascular Endothelial Niches Control Acute T Cell Leukemia Maintenance,” Cancer Cell 27:755-768). These are the tumor locations not only for T-LL but also for AITL.

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin. M-protein, short for monoclonal protein, also known as paraprotein, is a particularly abnormal protein produced by the myeloma plasma cells and can be found in the blood or urine of almost all patients with multiple myeloma.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

Bone marrow stromal cells are well known to support multiple myeloma disease progression and resistance to chemotherapy. Disrupting the interactions between multiple myeloma cells and stromal cells is an additional target of multiple myeloma chemotherapy.

Myelodysplastic syndrome (MDS) refers to a diverse group of hematopoietic stem cell disorders. MDS can be characterized by a cellular marrow with impaired morphology and maturation (dysmyelopoiesis), ineffective blood cell production, or hematopoiesis, leading to low blood cell counts, or cytopenias, and high risk of progression to acute myeloid leukemia, resulting from ineffective blood cell production. See The Merck Manual 953 (17th ed. 1999) and List et al., 1990, J Clin. Oncol. 8:1424.

As a group of hematopoietic stem cell malignancies with significant morbidity and mortality, MDS is a highly heterogeneous disease, and the severity of symptoms and disease progression can vary widely among patients. The current standard clinical tool to evaluate risk stratification and treatment options is the revised International Prognostic Scoring System, or IPSS-R. The IPSS-R differentiates patients into five risk groups (Very Low, Low, Intermediate, High, Very High) based on evaluation of cytogenetics, percentage of blasts (undifferentiated blood cells) in the bone marrow, hemoglobin levels, and platelet and neutrophil counts. The WHO also suggested stratifying MDS patients by a del (5q) abnormality.

According to the ACS, the annual incidence of MDS is approximately 13,000 patients in the United States, the majority of which are 60 years of age or older. The estimated prevalence is over 60,000 patients in the United States. Approximately 75% of patients fall into the IPSS-R risk categories of Very Low, Low, and Intermediate, or collectively known as lower risk MDS.

The initial hematopoietic stem cell injury can be from causes such as, but not limited to, cytotoxic chemotherapy, radiation, virus, chemical exposure, and genetic predisposition. A clonal mutation predominates over bone marrow, suppressing healthy stem cells. In the early stages of MDS, the main cause of cytopenias is increased programmed cell death (apoptosis). As the disease progresses and converts into leukemia, gene mutation rarely occurs and a proliferation of leukemic cells overwhelms the healthy marrow. The disease course differs, with some cases behaving as an indolent disease and others behaving aggressively with a very short clinical course that converts into an acute form of leukemia.

An international group of hematologists, the French-American-British (FAB) Cooperative Group, classified MDS disorders into five subgroups, differentiating them from AML. The Merck Manual 954 (17^{th} ed. 1999); Bennett J. M., et al., Ann. Intern. Med. 1985 October, 103(4): 620-5; and Besa E. C., Med. Clin. North Am. 1992 May, 76(3): 599-617. An underlying trilineage dysplastic change in the bone marrow cells of the patients is found in all subtypes.

There are two subgroups of refractory anemia characterized by five percent or less myeloblasts in bone marrow: (1) refractory anemia (RA) and; (2) RA with ringed sideroblasts (RARS), defined morphologically as having 15% erythroid cells with abnormal ringed sideroblasts, reflecting an abnormal iron accumulation in the mitochondria. Both have a prolonged clinical course and low incidence of progression to acute leukemia. Besa E. C., Med. Clin. North Am. 1992 May, 76(3): 599-617.

There are two subgroups of refractory anemias with greater than five percent mycloblasts: (1) RA with excess blasts (RAEB), defined as 6-20% myeloblasts, and (2) RAEB in transformation (RAEB-T), with 21-30% myeloblasts. The higher the percentage of myeloblasts, the shorter the clinical course and the closer the disease is to acute myelogenous leukemia. Patient transition from early to more advanced stages indicates that these subtypes are merely stages of disease rather than distinct entities. Elderly patients with MDS with trilineage dysplasia and greater than 30% myeloblasts who progress to acute leukemia are often considered to have a poor prognosis because their response rate to chemotherapy is lower than de novo acute myeloid leukemia patients. The fifth type of MDS, the most difficult to classify, is CMML. This subtype can have any percentage of myeloblasts but presents with a monocytosis of 1000/dL or more. It may be associated with splenomegaly. This subtype overlaps with a myeloproliferative disorder and may have an intermediate clinical course. It is differentiated from the classic CML that is characterized by a negative Ph chromosome.

MDS is primarily a disease of elderly people, with the median onset in the seventh decade of life. The median age of these patients is 65 years, with ages ranging from the early third decade of life to as old as 80 years or older. The syndrome may occur in any age group, including the pediatric population. Patients who survive malignancy treatment with alkylating agents, with or without radiotherapy, have a high incidence of developing MDS or secondary acute leukemia. About 60-70% of patients do not have an obvious exposure or cause for MDS, and are classified as primary MDS patients.

The treatment of MDS is based on the stage and the mechanism of the disease that predominates the particular phase of the disease process. Bone marrow transplantation has been used in patients with poor prognosis or late-stage MDS. Epstein and Slease, 1985, Surg. Ann. 17:125. An alternative approach to therapy for MDS is the use of hematopoietic growth factors or cytokines to stimulate blood cell development in a recipient. Dexter, 1987, J. Cell Sci. 88:1; Moore, 1991, Annu. Rev. Immunol. 9:159; and Besa E. C., Med. Clin. North Am. 1992 May, 76(3): 599-617. The treatment of MDS using immunomodulatory compounds is described in U.S. Pat. No. 7,189,740, the entirety of which is hereby incorporated by reference.

Therapeutic options fall into three categories including supportive care, low intensity and high intensity therapy. Supportive care includes the use red blood cell and platelet transfusions and hematopoietic cytokines such as erythropoiesis stimulating agents or colony stimulating factors to improve blood counts. Low intensity therapies include hypomethylating agents such as azacytidine (Vidaza®) and decitabine (Dacogen®), biological response modifiers such as lenalidomide (Revlimid®), and immunosuppressive treatments such as cyclosporine A or antithymocyte globulin. High intensity therapies include chemotherapeutic agents such as idarubicin, azacytidine, fludarabine and topotecan, and hematopoietic stem cell transplants, or HSCT.

National Comprehensive Cancer Network, or NCCN, guidelines recommend that lower risk patients (IPSS-R groups Very Low, Low, Intermediate) receive supportive care or low intensity therapies with the major therapeutic goal of hematologic improvement, or HI. NCCN guidelines recommend that higher risk patients (IPSS-R groups High, Very High) receive more aggressive treatment with high intensity therapies. In some cases, high risk patients are unable to tolerate chemotherapy, and may elect lower intensity regimens. Despite currently available treatments, a substantial portion of MDS patients lack effective therapies and NCCN guidelines recommend clinical trials as additional therapeutic options. Treatment of MDS remains a significant unmet need requiring the development of novel therapies.

MPN is a group of diseases that affect blood-cell formation. In all forms of MPN, stem cells in the bone marrow develop genetic defects (called acquired defects) that cause them to grow and survive abnormally. This results in unusually high numbers of blood cells in the bone marrow (hypercellular marrow) and in the bloodstream. Sometimes in MPN, the abnormal stem cells cause scarring in the marrow, called myelofibrosis. Myelofibrosis can lead to low levels of blood cells, especially low levels of red blood cells (anemia). In MPN, the abnormal stem cells can also grow in the spleen, causing the spleen to enlarge (splenomegaly), and in other sites outside the marrow, causing enlargement of other organs.

There are several types of chronic MPN, based on the cells affected. Three classic types of MPN include polycythemia vera (PV), in which there are too many RBCs; essential thrombocythemia (ET), in which there are too many platelets; primary myelofibrosis (PMF), in which fibers and blasts (abnormal stem cells) build up in the bone marrow. Other types of MPN include: chronic myeloid leukemia, in which there are too many white blood cells; chronic neutrophilic leukemia, in which there are too many neutrophils; chronic eosinophilic leukemia, not otherwise specified, in which there are too many eosinophils (hypereosinophilia); mastocytosis, also called mast cell disease, in which there are too many mast cells, which are a type of immune system cell found in tissues, like skin and digestive organs, rather than in the bloodstream; myeloid and lymphoid neoplasms with eosinophilia and abnormalities of the PDGFRA, PDGFRB, and FGFR1 genes; and other unclassifiable myeloproliferative neoplasms.

Head and neck squamous cell carcinoma (HNSCC) is the 6^{th} most common cancer worldwide, with about 650,000 cases and 200,000 deaths per year worldwide, and about 54,000 new cases per year in the US. It is also the most common cancer in central Asia.

HNSCC has 2 different etiologies and corresponding tumor types. The first subtype is associated with tobacco smoking and alcohol consumption, and unrelated to Human papillomavirus (HPV− or HPV negative). The second subtype is associated with infection with high-risk HPV (HPV+ or HPV positive). The second subtype is largely limited to oropharyngeal cancers. HPV+ tumors are distinct entity with better prognosis and may require differential treatments.

As used herein, the term “treat,” “treating,” and “treatment,” when used in reference to a cancer patient, refer to an action that reduces the severity of the cancer, or retards or slows the progression of the cancer, including (a) inhibiting the cancer growth, or arresting development of the cancer, and (b) causing regression of the cancer, or delaying or minimizing one or more symptoms associated with the presence of the cancer.

As used herein, the term “determining” refers to using any form of measurement to assess the presence of a substance, either quantitatively or qualitatively. Measurement can be relative or absolute. Measuring the presence of a substance can include determining whether the substance is present or absent, or the amount of the substance.

As used herein, the term “analyzing” a sample refers to carrying that an art-recognized assay to make an assessment regarding a particular property or characteristic of the sample. The property or characteristic of the sample can be, for example, the type of the cells in the sample, or the presence of a mutation in a gene in the sample.

As used herein, the term “administer,” “administering,” or “administration” refers to the act of delivering, or causing to be delivered, a compound or a pharmaceutical composition to the body of a subject by a method described herein or otherwise known in the art. Administering a compound or a pharmaceutical composition includes prescribing a compound or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

A person of ordinary skill in the art would understand that clinical standards used to define CR, PR, or other level of patient responsiveness to treatments can vary for different subtypes of cancer. For example, for hematopoietic cancers, patient being “responsive” to a particular treatment can be defined as patients who have a complete response (CR), a partial response (PR), or hematological improvement (HI) (Lancet et al., Blood 2:2 (2006)). HI can be defined as any lymph node blast count less than 5% or a reduction in lymph node blasts by at least half. On the other hand, patient being “not responsive” to a particular treatment can be defined as patients who have either progressive disease (PD) or stable disease (SD). Progressive disease (PD) can be defined as either >50% increase in lymph node or circulating blast % from baseline, or new appearance of circulating blasts (on at least 2 consecutive occasions). Stable disease (SD) can be defined as any response not meeting CR, PR, HI, or PD criteria.

As used herein, the term “selecting” and “selected” in reference to a patient is used to mean that a particular patient is specifically chosen from a larger group of patients on the basis of (due to) the particular patient having a predetermined criteria or a set of predetermined criteria, e.g., a patient having a cancer characterized by or determined to have a mutation in a member of the KIR family. Similarly, “selectively treating a patient” refers to providing treatment to a patient who is specifically chosen from a larger group of patients on the basis of (due to) the particular patient having a predetermined criteria or a set of predetermined criteria, e.g., a mutation in a gene of the KIR family. Similarly, “selectively administering” refers to administering a drug to a patient having a cancer that is specifically chosen from a larger group of patients on the basis of (due to) the particular patient having a predetermined criteria or a set of predetermined criteria (e.g., a mutation in a gene of the KIR family). By selecting, selectively treating and selectively administering, it is meant that a patient is delivered a personalized therapy for a disease or disorder, e.g., cancer, based on the patient's biology, such as the disease or disorder in the selected patient being associated with a mutation in a gene of the KIR family, rather than being delivered a standard treatment regimen based solely on having the disease or disorder (e.g., a leukemia).

As used herein, the term “therapeutically effective amount” of a compound when used in connection with a disease or disorder refers to an amount sufficient to provide a therapeutic benefit in the treatment or management of the disease or disorder or to delay or minimize one or more symptoms associated with the disease or disorder. A therapeutically effective amount of a compound means an amount of the compound that when used alone or in combination with other therapies, would provide a therapeutic benefit in the treatment or management of the disease or disorder. The term encompasses an amount that improves overall therapy, reduces or avoids symptoms, or enhances the therapeutic efficacy of another therapeutic agent. The term also refers to the amount of a compound that sufficiently elicits the biological or medical response of a biological molecule (e.g., a protein, enzyme, RNA, or DNA), cell, tissue, system, animal, or human, which is being sought by a researcher, veterinarian, medical doctor, or clinician.

As used herein, the term “express” or “expression” when used in connection with a gene refers to the process by which the information carried by the gene becomes manifest as the phenotype, including transcription of the gene to a messenger RNA (mRNA), the subsequent translation of the mRNA molecule to a polypeptide chain and its assembly into the ultimate protein.

As used herein, the term “expression level” of a biomarker refers to the amount or accumulation of the expression product of a biomarker, such as, for example, the amount of a RNA product of the biomarker (the RNA level of the biomarker) or the amount of a protein product of the biomarker (the protein level of the biomarker). If the biomarker is a gene with more than one alleles, the expression level of a biomarker refers to the total amount of accumulation of the expression product of all existing alleles for this gene, unless otherwise specified.

As used herein, the term “biomarker” refers to a gene or a mutation in a gene that can be either present or absent in individual subjects. The presence a biomarker in a sample from a subject can indicate the responsiveness of the subject to a particular treatment, such as an FTI treatment.

As used herein, the term “responsiveness” or “responsive” when used in connection with a treatment refers to the effectiveness of the treatment in lessening or decreasing the symptoms of the disease being treated. For example, a cancer patient is responsive to an FTI treatment if the FTI treatment effectively inhibits the cancer growth, or arrests development of the cancer, causes regression of the cancer, or delays or minimizes one or more symptoms associated with the presence of the cancer in this patient.

The responsiveness to a particular treatment of a cancer patient can be characterized as a complete or partial response. “Complete response,” or “CR” refers to an absence of clinically detectable disease with normalization of previously abnormal radiographic studies, bone marrow, and cerebrospinal fluid (CSF) or abnormal monoclonal protein measurements. “Partial response,” or “PR,” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions.

A person of ordinary skill in the art would understand that clinical standards used to define CR, PR, or other level of patient responsiveness to treatments can vary for different types of cancer. For example, for hematopoietic cancers, patient being “responsive” to a particular treatment can be defined as patients who have a complete response (CR), a partial response (PR), or hematological improvement (HI) (Lancet et al., Blood 2:2 (2006)). HI can be defined as any bone marrow blast count less than 5% or a reduction in bone marrow blasts by at least half. On the other hand, patient being “not responsive” to a particular treatment can be defined as patients who have either progressive disease (PD) or stable disease (SD). Progressive disease (PD) can be defined as either >50% increase in bone marrow or circulating blast % from baseline, or new appearance of circulating blasts (on at least 2 consecutive occasions). Stable disease (SD) can be defined as any response not meeting CR, PR, HI, or PD criteria.

As used herein, the term “likelihood” refers to the probability of an event. A subject is “likely” to be responsive to a particular treatment when a condition is met means that the probability of the subject to be responsive to a particular treatment is higher when the condition is met than when the condition is not met. The probability to be responsive to a particular treatment can be higher by, for example, 5%, 10%, 25%, 50%, 100%, 200%, or more in a subject who meets a particular condition compared to a subject who does not meet the condition.

As used herein, the term “NK cell,” or “natural killer cell,” refers to the type of bone marrow-derived large granular lymphocytes that share a common progenitor with T cells, but do not have B cell or T cell surface markers. NK cells usually constitute 10-15% of all circulating lymphocytes. NK cells are defensive cells of innate immunity that recognize structures on the surface of virally infected cells or tumor cells and kill these cells by releasing cytotoxins. NK cells can be activated without previous antigen exposure.

In order to kill infected cells or tumor cells selectively, NK cells must distinguish healthy cells from diseased cells. The cytolytic activity of human NK cells is modulated by the interaction of inhibitory and activatory membrane receptors, which are expressed on the surface of NK cells, with MHC (HLA) class I molecules, which are expressed by non-NK cells, including tumor cells, or cells from a bone marrow transplant recipient. The killer cell immunoglobulin-like receptors (KIR; or CD158) mapping to chromosome 19q13.4.3-5, constitute a family of MHC-I (HLA-A, -B, -C) binding receptors that regulate the activation threshold of NK cells (Valiante el at. Immunity 7:739-751(1997)).

In humans, the class I HLA complex is about 2000 kb long and contains about 20 genes. Within the class I region exist genes encoding the well characterized class I MHC molecules designated HLA-A, HLA-B and HLA-C. In addition, there are nonclassical class I genes that include HLA-E, HLA-F, HLA-G, HLA-H, HLA-J and HLA-X as well as a new family known as MIC. While HLA-A and -B play some role, the interactions between KIRs and HLA-C molecules predominate in preventing NK cells from attacking healthy autologous cells (Colonna et al. PNAS, 90:1200-12004 (1993); Moesta A K et al., Front Immunol. 3:336 (2012)).

As used herein, the term “KIR genes” refers to the genes that encode the KIR receptors on NK cells. The KIR genes are clustered in one of the most variable regions of the human genome in terms of both gene content and sequence polymorphism. This extensive variability generates a repertoire of NK cells in which KIR receptors are expressed at the cell surface in a combinatorial fashion. KIR receptors are transmembrane glycoproteins expressed on the plasma membrane of NK cells and a subset of T cells. Interactions between the KIR receptors and their appropriate ligands on target cells result in the production of positive or negative signals that regulate NK cell function.

To date, at least 14 distinct KIR genes have been identified, which are KIR2DL1, KIR2DL2, KIR2DL3, KIR2DL4, KIR2DL5, KIR2DS1, KIR2DS2, KIR2DS3, KIR2DS4, KIR2DS5, KIR3DL1, KIR3DL2, KIR3DL3, KIR3DS1. These genes share extensive sequence homology. Each gene is about 9-16 Kb in length, divided into 8-9 exons that encode the signal peptide, two or three extracellular domains, stem, transmembrane region, and cytoplasmic tail (sometimes referred to as cytoplasmic domain). Nomenclature of KIRs is based on the number of their extracellular Ig-like domains (2D or 3D) and the length of their cytoplasmic tail (long (L) or short (S)). For example, killer cell immunoglobulin like receptor, two Ig domains and long cytoplasmic tail 1 is referred to as KIR2DL1. These genes vary with respect to their presence or absence on different KIR haplotypes, creating considerable diversity in the number of KIR genotypes observed in the population. For example, some individuals might carry only seven of the 14 KIR genes while other individuals might carry 12 of the 14 KIR genes. One particular KIR gene can have multiple alleles. Each KIR gene encodes either an inhibitory or an activating KIR. For example, KIR2DL4 is considered an activating KIR (though KIR2DL4 does have some inhibitory capabilities), and KIR2DL1, KIR2DL3, KIR3DL1, and KIR3DL2 and each considered inhibitory KIRs.

In terms of KIR signalling, with the exception of KIR2DL4, which has both activating and inhibitory capabilities, KIR receptors with long cytoplasmic tails (L) are considered inhibitory KIRs while those with short tails (S) are considered activating KIRs. KIR inhibitory receptors signal through immunoreceptor tyrosine-based inhibitory motif (ITIM) in their cytoplasmic domain. When inhibitory KIR receptors bind to a ligand, their ITIMs are tyrosine phosphorylated and protein tyrosine phosphatases, including SHP-1, are recruited. Activating receptors do not have ITIM, but instead contain a positively charged lysine or arginine residue in their transmembrane domain that helps to bind DAP12, an adaptor molecule containing an immunoreceptor tyrosine-based activation motifs (ITAM). ITAMs allow the docking and activation of SRC and SYK.

An exemplary amino acid sequence and a corresponding encoding nucleic acid sequence of human KIR2DL1 (GENBANK: SPC71652.1; NM_014218.3) are provided below:

| (SEQ ID NO.: 1) | |
|---|---|
| 1 | MSLLVVSMAC VGFFLLQGAW PHEGVHRKPS LLAHPGRLVK SEETVILQCW SDVMFEHFLL |
| 61 | HREGMFNDTL RLIGEHHDGV SKANFSISRM TQDLAGTYRC YGSVTHSPYQ VSAPSDPLDI |
| 121 | VIIGLYEKPS LSAQLGPTVL AGENVTLSCS SRSSYDMYHL SREGEAHERR LPAGPKVNGT |
| 181 | FQADFPLGPA THGGTYRCFG SFHDSPYEWS KSSDPLLVSV TGNPSNSWPS PTEPSSKTGN |
| 241 | PRHLHILIGT SVVIILFILL FFLLHHWCSN KKNAAVMDQE SAGNRTANSE DSDEQDPQEV |
| 301 | TYTQLNHCVF TQRKITRPSQ RPKTPPTDII VYTELPNAES RSKVVSCP |
| (SEQ ID NO.: 2) | |
| 1 | ATCCTGTGCG CTGCTGAGCT GAGCTCGGTC GCGGCTGCCT GTCTGCTCCG GCAGCACCAT |
| 61 | GTCGCTCTTG GTCGTCAGCA TGGCGTGTGT TGGGTTCTTC TTGCTGCAGG GGGCCTGGCC |
| 121 | ACATGAGGGA GTCCACAGAA AACCTTCCCT CCTGGCCCAC CCAGGTCGCC TGGTGAAATC |
| 181 | AGAAGAGACA GTCATCCTGC AGTGTTGGTC AGATGTCATG TTTGAACACT TCCTTCTGCA |
| 241 | CAGAGAGGGG ATGTTTAACG ACACTTTGCG CCTCATTGGA GAACACCATG ATGGGGTCTC |
| 301 | CAAGGCCAAC TTCTCCATCA GTCGCATGAC GCAAGACCTG GCAGGGACCT ACAGATGCTA |
| 361 | CGGTTCTGTT ACTCACTCCC CCTATCAGGT GTCAGCTCCC AGTGACCCTC TGGACATCGT |
| 421 | GATCATAGGT CTATATGAGA AACCTTCTCT CTCAGCCCAG CTGGGCCCCA CGGTTCTGGC |
| 481 | AGGAGAGAAT GTGACCTTGT CCTGCAGCTC CCGGAGCTCC TATGACATGT ACCATCTATC |
| 541 | CAGGGAAGGG GAGGCCCATG AACGTAGGCT CCCTGCAGGG CCCAAGGTCA ACGGAACATT |
| 601 | CCAGGCTGAC TTTCCTCTGG GCCCTGCCAC CCACGGAGGG ACCTACAGAT GCTTCGGCTC |
| 661 | TTTCCATGAC TCTCCATACG AGTGGTCAAA GTCAAGTGAC CCACTGCTTG TTTCTGTCAC |
| 721 | AGGAAACCCT TCAAATAGTT GGCCTTCACC CACTGAACCA AGCTCCAAAA CCGGTAACCC |
| 781 | CCGACACCTG CACATTCTGA TTGGGACCTC AGTGGTCATC ATCCTCTTCA TCCTCCTCTT |
| 841 | CTTTCTCCTT CATCGCTGGT GCTCCAACAA AAAAAATGCT GCGGTAATGG ACCAAGAGTC |
| 901 | TGCAGGAAAC AGAACAGCGA ATAGCGAGGA CTCTGATGAA CAAGACCCTC AGGAGGTGAC |
| 961 | ATACACACAG TTGAATCACT GCGTTTTCAC ACAGAGAAAA ATCACTCGCC CTTCTCAGAG |
| 1021 | GCCCAAGACA CCCCCAACAG ATATCATCGT GTACACGGAA CTTCCAAATG CTGAGTCCAG |
| 1081 | ATCCAAAGTT GTCTCCTGCC CATGAGCACC ACAGTCAGGC CTTGAGGGCG TCTTCTAGGG |
| 1141 | AGACAACAGC CCTGTCTCAA AACCGGGTTG CCAGCTCCCA TGTACCAGCA GCTGGAATCT |
| 1201 | GAAGGCGTGA GTCTGCATCT TAGGGCATCG ATCTTCCTCA CACCACAAAT CTGAATGTGC |
| 1261 | CTCTCTCTTG CTTACAAATG TCTAAGGTCC CCACTGCCTG CTGGAGAAAA AACACACTCC |
| 1321 | TTTGCTTAAC CCACAGTTCT CCATTTCACT TGACCCCTGC CCACCTCTCC AACCTAACTG |
| 1381 | GCTTACTTCC TAGTCTACTT GAGGCTGCAA TCACACTGAG GAACTCACAA TTCCAAACAT |
| 1441 | ACAAGAGGCT CCCTCTTAAC GCAGCACTTA GACACGTGTT GTTCCACCTT CCCTCATGCT |
| 1501 | GTTCCACCTC CCCTCAGACT AGCTTTCAGT CTTCTGTCAG CAGTAAAACT TATATATTTT |
| 1561 | TTAAAATAAC TTCAATGTAG TTTTCCATCC TTCAAATAAA CATGTCTGCC CCCA |

An exemplary amino acid sequence and a corresponding encoding nucleic acid sequence of human KIR2DL3 (GENBANK: NP_056952.2; NM_015868.2) are provided below:

| (SEQ ID NO.: 3) | |
|---|---|
| 1 | MSLMVVSMVC VGFFLLQGAW PHEGVHRKPS LLAHPGPLVK SEETVILQCW SDVRFQHFLL |
| 61 | HREGKFKDTL HLIGEHHDGV SKANFSIGPM MQDLAGTYRC YGSVTHSPYQ LSAPSDPLDI |
| 121 | VITGLYEKPS LSAQPGPTVL AGESVTLSCS SRSSYDMYHL SREGEAHERR FSAGPKVNGT |
| 181 | FQADFPLGPA THGGTYRCFG SFRDSPYEWS NSSDPLLVSV TGNPSNSWPS PTEPSSETGN |
| 241 | PRHLHVLIGT SVVIILFILL LFFLLHRWCC NKKNAVVMDQ EPAGNRTVNR EDSDEQDPQE |
| 301 | VTYAQLNHCV FTQRKITRPS QRPKTPPTDI IVYTELPNAE P |
| (SEQ ID NO.: 4) | |
| 1 | AGCTGGGGCG CGGCCGCCTG TCTGCACAGA CAGCACCATG TCGCTCATGG TCGTCAGCAT |
| 61 | GGTGTGTGTT GGGTTCTTCT TGCTGCAGGG GGCCTGGCCA CATGAGGGAG TCCACAGAAA |
| 121 | ACCTTCCCTC CTGGCCCACC CAGGTCCCCT GGTGAAATCA GAAGAGACAG TCATCCTGCA |
| 181 | ATGTTGGTCA GATGTCAGGT TTCAGCACTT CCTTCTGCAC AGAGAAGGGA AGTTTAAGGA |
| 241 | CACTTTGCAC CTCATTGGAG AGCACCATGA TGGGGTCTCC AAGGCCAACT TCTCCATCGG |
| 301 | TCCCATGATG CAAGACCTTG CAGGGACCTA CAGATGCTAC GGTTCTGTTA CTCACTCCCC |
| 361 | CTATCAGTTG TCAGCTCCCA GTGACCCTCT GGACATCGTC ATCACAGGTC TATATGAGAA |
| 421 | ACCTTCTCTC TCAGCCCAGC CGGGCCCCAC GGTTCTGGCA GGAGAGAGCG TGACCTTGTC |
| 481 | CTGCAGCTCC CGGAGCTCCT ATGACATGTA CCATCTATCC AGGGAGGGGG AGGCCCATGA |
| 541 | ACGTAGGTTC TCTGCAGGGC CCAAGGTCAA CGGAACATTC CAGGCCGACT TTCCTCTGGG |
| 601 | CCCTGCCACC CACGGAGGAA CCTACAGATG CTTCGGCTCT TTCCGTGACT CTCCATACGA |
| 661 | GTGGTCAAAC TCGAGTGACC CACTGCTTGT TTCTGTCACA GGAAACCCTT CAAATAGTTG |
| 721 | GCCTTCACCC ACTGAACCAA GCTCCGAAAC CGGTAACCCC AGACACCTGC ATGTTCTGAT |
| 781 | TGGGACCTCA GTGGTCATCA TCCTCTTCAT CCTCCTCCTC TTCTTTCTCC TTCATCGCTG |
| 841 | GTGCTGCAAC AAAAAAAATG CTGTTGTAAT GGACCAAGAG CCTGCAGGGA ACAGAACAGT |
| 901 | GAACAGGGAG GACTCTGATG AACAAGACCC TCAGGAGGTG ACATATGCAC AGTTGAATCA |
| 961 | CTGCGTTTTC ACACAGAGAA AAATCACTCG CCCTTCTCAG AGGCCCAAGA CACCCCCAAC |
| 1021 | AGATATCATC GTGTACACGG AACTTCCAAA TGCTGAGCCC TGATCCAAAG TTGTCTCCTG |
| 1081 | CCCATGAGCA CCACAGTCAG GCCTTGAGGG GATCTTCTAG GGAGACAACA GCCCTGTCTC |
| 1141 | AAAACTGGGT TGCCAGCTCC AATGTACCAG CAGCTGGAAT CTGAAGGCGT GAGTCTGCAT |
| 1201 | CTTAGGGCAT CGCTCTTCCT CACACCACAA ATCTGAACGT GCCTCTCCCT TGCTTACAAA |
| 1261 | TGTCTAAGGT CCCCACTGCC TGCTGGAGAG AAAACACACT CCTTTGCTTA GCCCACAATT |
| 1321 | CTCCATTTCA CTTGACCCCT GCCCACCTCT CCAACCTAAC TGGCTTACTT CCTAGTCTAC |
| 1381 | TTGAGGCTGC AATCACACTG AGGAACTCAC AATTCCAAAC ATACAAGAGG CTCCCTCTTA |
| 1441 | ACACGGCACT TAGACACGTG CTGTTCCACC TTCCCTCATG CTGTTCCACC TCCCCTCAGA |
| 1501 | CTAGCTTTCA GCCTTCTGTC AGCAGTAAAA CTTATATATT TTTTAAAATA ATTTCAATGT |
| 1561 | AGTTTTCCCT CCTTCAAATA AACATGTCTG CCCTCA |

An exemplary amino acid sequence and a corresponding encoding nucleic acid sequence of human KIR2DL4 (GENBANK: NP_002246.5; NM_002255.6) are provided below:

| (SEQ ID NO.: 5) | |
|---|---|
| 1 | MSMSPTVIIL ACLGFFLDQS VWAHVGGQDK PFCSAWPSAV VPQGGHVTLR CHYRRGFNIF |
| 61 | TLYKKDGVPV PELYNRIFWN SFLISPVTPA HAGTYRCRGF HPHSPTEWSA PSNPLVIMVT |
| 121 | GLYEKPSLTA RPGPTVRAGE NVTLSCSSQS SFDIYHLSRE GEAHELRLPA VPSINGTFQA |
| 181 | DFPLGPATHG ETYRCFGSFH GSPYEWSDPS DPLPVSVTGN PSSSWPSPTE PSFKTGIARH |
| 241 | LHAVIRYSVA IILFTILPFF LLHRWCSKKK NAAVMNQEPA GHRTVNREDS DEQDPQEVTY |
| 301 | AQLDHCIFTQ RKITGPSQRS KRPSTDTSVC IELPNAEPRA LSPAHEHHSQ ALMGSSRETT |
| 361 | ALSQTQLASS NVPAAGI |
| (SEQ ID NO.: 6) | |
| 1 | AGTCGAGCCG AGTCACTGCG TCCTGGCAGC AGAAGCTGCA CCATGTCCAT GTCACCCACG |
| 61 | GTCATCATCC TGGCATGTCT TGGGTTCTTC TTGGACCAGA GTGTGTGGGC ACACGTGGGT |
| 121 | GGTCAGGACA AGCCCTTCTG CTCTGCCTGG CCCAGCGCTG TGGTGCCTCA AGGAGGACAC |
| 181 | GTGACTCTTC GGTGTCACTA TCGTCGTGGG TTTAACATCT TCACGCTGTA CAAGAAAGAT |
| 241 | GGGGTCCCTG TCCCTGAGCT CTACAACAGA ATATTCTGGA ACAGTTTCCT CATTAGCCCT |
| 301 | GTGACCCCAG CACACGCAGG GACCTACAGA TGTCGAGGTT TTCACCCGCA CTCCCCCACT |
| 361 | GAGTGGTCGG CACCCAGCAA CCCCCTGGTG ATCATGGTCA CAGGTCTATA TGAGAAACCT |
| 421 | TCGCTTACAG CCCGGCCGGG CCCCACGGTT CGCGCAGGAG AGAACGTGAC CTTGTCCTGC |
| 481 | AGCTCCCAGA GCTCCTTTGA CATCTACCAT CTATCCAGGG AGGGGGAAGC CCATGAACTT |
| 541 | AGGCTCCCTG CAGTGCCCAG CATCAATGGA ACATTCCAGG CCGACTTCCC TCTGGGTCCT |
| 601 | GCCACCCACG GAGAGACCTA CAGATGCTTC GGCTCTTTCC ATGGATCTCC CTACGAGTGG |
| 661 | TCAGACCCGA GTGACCCACT GCCTGTTTCT GTCACAGGAA ACCCTTCTAG TAGTTGGCCT |
| 721 | TCACCCACTG AACCAAGCTT CAAAACTGGT ATCGCCAGAC ACCTGCATGC TGTGATTAGG |
| 781 | TACTCAGTGG CCATCATCCT CTTTACCATC CTTCCCTTCT TTCTCCTTCA TCGCTGGTGC |
| 841 | TCCAAAAAAA AAAATGCTGC TGTAATGAAC CAAGAGCCTG CGGGACACAG AACAGTGAAC |
| 901 | AGGGAGGACT CTGATGAACA AGACCCTCAG GAGGTGACAT ACGCACAGTT GGATCACTGC |
| 961 | ATTTTCACAC AGAGAAAAAT CACTGGCCCT TCTCAGAGGA GCAAGAGACC CTCAACAGAT |
| 1021 | ACCAGCGTGT GTATAGAACT TCCAAATGCT GAGCCCAGAG CGTTGTCTCC TGCCCATGAG |
| 1081 | CACCACAGTC AGGCCTTGAT GGGATCTTCT AGGGAGACAA CAGCCCTGTC TCAAACCCAG |
| 1141 | CTTGCCAGCT CTAATGTACC AGCAGCTGGA ATCTGAAGGC GTGAGTCTCC ATCTTAGAGC |
| 1201 | ATCACTCTTC CTCACACCAC AAATCTGGTG CCTGTCTCTT GCTTACCAAT GTCTAAGGTC |
| 1261 | CCCACTGCCT GCTGCAGAGA AAACACACTC CTTTGCTTAG CCCACAATTC TCTATTTCAC |
| 1321 | TTGACCCCTG CCCACCTCTC CAACCTAACT GGCTTACTTC CTAGTCTACT TGAGGCTGCA |
| 1381 | ATCACACTGA GGAACTCACA ATTCCAAACA TACAAGAGGC TCTCTCTTAA CACGGCACTT |
| 1441 | AGACACGTGC TGTTCCACCT TCCCTCGTGC TGTTCCACCT TTCCTCAGAC TATTTTTCAG |
| 1501 | CCTTCTGGCA TCAGCAAACC TTATAAAATT TTTTTGATTT CAGTGTAGTT CTCTCCTCTT |
| 1561 | CAAATAAACA TGTCTGCCTT CA |

An exemplary amino acid sequence and a corresponding encoding nucleic acid sequence of human KIR3DL1 (GENBANK: NP_037421.2; NM_013289.2) are provided below:

| (SEQ ID NO.: 7) | |
|---|---|
| 1 | MSLMVVSMAC VGLFLVQRAG PHMGGQDKPF LSAWPSAVVP RGGHVTLRCH YRHRFNNFML |
| 61 | YKEDRIHIPI FHGRIFQESF NMSPVTTAHA GNYTCRGSHP HSPTGWSAPS NPVVIMVTGN |
| 121 | HRKPSLLAHP GPLVKSGERV ILQCWSDIMF EHFFLHKEGI SKDPSRLVGQ IHDGVSKANF |
| 181 | SIGPMMLALA GTYRCYGSVT HTPYQLSAPS DPLDIVVTGP YEKPSLSAQP GPKVQAGESV |
| 241 | TLSCSSRSSY DMYHLSREGG AHERRLPAVR KVNRTFQADF PLGPATHGGT YRCFGSFRHS |
| 301 | PYEWSDPSDP LLVSVTGNPS SSWPSPTEPS SKSGNPRHLH ILIGTSVVII LFILLLFFLL |
| 361 | HLWCSNKKNA AVMDQEPAGN RTANSEDSDE QDPEEVTYAQ LDHCVFTQRK ITRPSQRPKT |
| 421 | PPTDTILYTE LPNAKPRSKV VSCP |
| (SEQ ID NO.: 8) | |
| 1 | ATAACATCCT GTGCGCTGCT GAGCTGAGCT GGGGCGCAGC CGCCTGTCTG CACCGGCAGC |
| 61 | ACCATGTCGC TCATGGTCGT CAGCATGGCG TGTGTTGGGT TGTTCTTGGT CCAGAGGGCC |
| 121 | GGTCCACACA TGGGTGGTCA GGACAAACCC TTCCTGTCTG CCTGGCCCAG CGCTGTGGTG |
| 181 | CCTCGAGGAG GACACGTGAC TCTTCGGTGT CACTATCGTC ATAGGTTTAA CAATTTCATG |
| 241 | CTATACAAAG AAGACAGAAT CCACATTCCC ATCTTCCATG GCAGAATATT CCAGGAGAGC |
| 301 | TTCAACATGA GCCCTGTGAC CACAGCACAT GCAGGGAACT ACACATGTCG GGGTTCACAC |
| 361 | CCACACTCCC CCACTGGGTG GTCGGCACCC AGCAACCCCG TGGTGATCAT GGTCACAGGA |
| 421 | AACCACAGAA AACCTTCCCT CCTGGCCCAC CCAGGTCCCC TGGTGAAATC AGGAGAGAGA |
| 481 | GTCATCCTGC AATGTTGGTC AGATATCATG TTTGAGCACT TCTTTCTGCA CAAAGAGGGG |
| 541 | ATCTCTAAGG ACCCCTCACG CCTCGTTGGA CAGATCCATG ATGGGGTCTC CAAGGCCAAT |
| 601 | TTCTCCATCG GTCCCATGAT GCTTGCCCTT GCAGGGACCT ACAGATGCTA CGGTTCTGTT |
| 661 | ACTCACACCC CCTATCAGTT GTCAGCTCCC AGTGATCCCC TGGACATCGT GGTCACAGGT |
| 721 | CCATATGAGA AACCTTCTCT CTCAGCCCAG CCGGGCCCCA AGGTTCAGGC AGGAGAGAGC |
| 781 | GTGACCTTGT CCTGTAGCTC CCGGAGCTCC TATGACATGT ACCATCTATC CAGGGAGGGG |
| 841 | GGAGCCCATG AACGTAGGCT CCCTGCAGTG CGCAAGGTCA ACAGAACATT CCAGGCAGAT |
| 901 | TTCCCTCTGG GCCCTGCCAC CCACGGAGGG ACCTACAGAT GCTTCGGCTC TTTCCGTCAC |
| 961 | TCTCCCTACG AGTGGTCAGA CCCGAGTGAC CCACTGCTTG TTTCTGTCAC AGGAAACCCT |
| 1021 | TCAAGTAGTT GGCCTTCACC CACAGAACCA AGCTCCAAAT CTGGTAACCC CAGACACCTG |
| 1081 | CACATTCTGA TTGGGACCTC AGTGGTCATC ATCCTCTTCA TCCTCCTCCT CTTCTTTCTC |
| 1141 | CTTCATCTCT GGTGCTCCAA CAAAAAAAAT GCTGCTGTAA TGGACCAAGA GCCTGCAGGG |
| 1201 | AACAGAACAG CCAACAGCGA GGACTCTGAT GAACAAGACC CTGAGGAGGT GACATACGCA |
| 1261 | CAGTTGGATC ACTGCGTTTT CACACAGAGA AAAATCACTC GCCCTTCTCA GAGGCCCAAG |
| 1321 | ACACCCCCTA CAGATACCAT CTTGTACACG GAACTTCCAA ATGCTAAGCC CAGATCCAAA |
| 1381 | GTTGTCTCCT GCCCATGAGC ACCACAGTCA GGCCTTGAGG ACGTCTTCTA GGGAGACAAC |
| 1441 | AGCCCTGTCT CAAAACCGAG TTGCCAGCTC CCATGTACCA GCAGCTGGAA TCTGAAGGCG |
| 1501 | TGAGTCTTCA TCTTAGGGCA TCGCTCCTCC TCACGCCACA AATCTGGTGC CTCTCTCTTG |
| 1561 | CTTACAAATG TCTAGGTCCC CACTGCCTGC TGGAAAGAAA ACACACTCCT TTGCTTAGCC |
| 1621 | CACAGTTCTC CATTTCACTT GACCCCTGCC CACCTCTCCA ACCTAACTGG CTTACTTCCT |
| 1681 | AGTCTACTTG AGGCTGCAAT CACACTGAGG AACTCACAAT TCCAAACATA CAAGAGGCTC |
| 1741 | CCTCTTGACG TGGCACTTAC CCACGTGCTG TTCCACCTTC CCTCATGCTG TTTCACCTTT |
| 1801 | CTTCGGACTA TTTTCCAGCC TTCTGTCAGC AGTGAAACTT ATAAAATTTT TTGTGATTTC |
| 1861 | AATGTAGCTG TCTCCTCTTC AAATAAACAT GTCTGCCCTC AAAAAAAAAA AAAAAAAAAA |
| 1921 | AAAAAAAAAA AAAAAAAAAA AAAAAAAAAA AAAAAAAAAA AAAAAAAAAA AAAAAAAAAA |
| 1981 | AAAAAA |

An exemplary amino acid sequence and a corresponding encoding nucleic acid sequence of human KIR3DL2 (GENBANK: NP_006728.2; NM_006737.3) are provided below:

| (SEQ ID NO.: 9) | |
|---|---|
| 1 | MSLTVVSMAC VGFFLLQGAW PLMGGQDKPF LSARPSTVVP RGGHVALQCH YRRGFNNFML |
| 61 | YKEDRSHVPI FHGRIFQESF IMGPVTPAHA GTYRCRGSRP HSLTGWSAPS NPLVIMVTGN |
| 121 | HRKPSLLAHP GPLLKSGETV ILQCWSDVMF EHFFLHREGI SEDPSRLVGQ IHDGVSKANF |
| 181 | SIGPLMPVLA GTYRCYGSVP HSPYQLSAPS DPLDIVITGL YEKPSLSAQP GPTVQAGENV |
| 241 | TLSCSSWSSY DIYHLSREGE AHERRLRAVP KVNRTFQADF PLGPATHGGT YRCFGSFRAL |
| 301 | PCVWSNSSDP LLVSVTGNPS SSWPSPTEPS SKSGICRHLH VLIGTSVVIF LFILLLFFLL |
| 361 | YRWCSNKKNA AVMDQEPAGD RTVNRQDSDE QDPQEVTYAQ LDHCVFIQRK ISRPSQRPKT |
| 421 | PLTDTSVYTE LPNAEPRSKV VSCPRAPQSG LEGVF |
| (SEQ ID NO.: 10) | |
| 1 | GGGGCGCGGC CTCCTGTCTG CACCGGCAGC ACCATGTCGC TCACGGTCGT CAGCATGGCG |
| 61 | TGCGTTGGGT TCTTCTTGCT GCAGGGGGCC TGGCCACTCA TGGGTGGTCA GGACAAACCC |
| 121 | TTCCTGTCTG CCCGGCCCAG CACTGTGGTG CCTCGAGGAG GACACGTGGC TCTTCAGTGT |
| 181 | CACTATCGTC GTGGGTTTAA CAATTTCATG CTGTACAAAG AAGACAGAAG CCACGTTCCC |
| 241 | ATCTTCCACG GCAGAATATT CCAGGAGAGC TTCATCATGG GCCCTGTGAC CCCAGCACAT |
| 301 | GCAGGGACCT ACAGATGTCG GGGTTCACGC CCACACTCCC TCACTGGGTG GTCGGCACCC |
| 361 | AGCAACCCCC TGGTGATCAT GGTCACAGGA AACCACAGAA AACCTTCCCT CCTGGCCCAC |
| 421 | CCAGGGCCCC TGCTGAAATC AGGAGAGACA GTCATCCTGC AATGTTGGTC AGATGTCATG |
| 481 | TTTGAGCACT TCTTTCTGCA CAGAGAGGGG ATCTCTGAGG ACCCCTCACG CCTCGTTGGA |
| 541 | CAGATCCATG ATGGGGTCTC CAAGGCCAAC TTCTCCATCG GTCCCTTGAT GCCTGTCCTT |
| 601 | GCAGGAACCT ACAGATGTTA TGGTTCTGTT CCTCACTCCC CCTATCAGTT GTCAGCTCCC |
| 661 | AGTGACCCCC TGGACATCGT GATCACAGGT CTATATGAGA AACCTTCTCT CTCAGCCCAG |
| 721 | CCGGGCCCCA CGGTTCAGGC AGGAGAGAAC GTGACCTTGT CCTGTAGCTC CTGGAGCTCC |
| 781 | TATGACATCT ACCATCTGTC CAGGGAAGGG GAGGCCCATG AACGTAGGCT CCGTGCAGTG |
| 841 | CCCAAGGTCA ACAGAACATT CCAGGCAGAC TTTCCTCTGG GCCCTGCCAC CCACGGAGGG |
| 901 | ACCTACAGAT GCTTCGGCTC TTTCCGTGCC CTGCCCTGCG TGTGGTCAAA CTCAAGTGAC |
| 961 | CCACTGCTTG TTTCTGTCAC AGGAAACCCT TCAAGTAGTT GGCCTTCACC CACAGAACCA |
| 1021 | AGCTCCAAAT CTGGTATCTG CAGACACCTG CATGTTCTGA TTGGGACCTC AGTGGTCATC |
| 1081 | TTCCTCTTCA TCCTCCTCCT CTTCTTTCTC CTTTATCGCT GGTGCTCCAA CAAAAAGAAT |
| 1141 | GCTGCTGTAA TGGACCAAGA GCCTGCGGGG GACAGAACAG TGAATAGGCA GGACTCTGAT |
| 1201 | GAACAAGACC CTCAGGAGGT GACGTACGCA CAGTTGGATC ACTGCGTTTT CATACAGAGA |
| 1261 | AAAATCAGTC GCCCTTCTCA GAGGCCCAAG ACACCCCTAA CAGATACCAG CGTGTACACG |
| 1321 | GAACTTCCAA ATGCTGAGCC CAGATCCAAA GTTGTCTCCT GCCCACGAGC ACCACAGTCA |
| 1381 | GGTCTTGAGG GGGTTTTCTA GGGAGACAAC AGCCCTGTCT CAAAACCAGG TTGCCAGATC |
| 1441 | CAATGAACCA GCAGCTGGAA TCTGAAGGCA TCAGTCTGCA TCTTAGGGGA TCGCTCTTCC |
| 1501 | TCACACCACG AATCTGAACA TGCCTCTCTC TTGCTTACAA ATGCCTAAGG TCGCCACTGC |
| 1561 | CTGCTGCAGA GAAAACACAC TCCTTTGCTT AGCCCACAAG TATCTATTTC ACTTGACCCC |
| 1621 | TGCCCACCTC TCCAACCTAA CTGGCTTACT TCCTAGTCCT ACTTGAGGCT GCAATCACAC |
| 1681 | TGAGGAACTC ACAATTCCAA ACATACAAGA GGCTCCCTCT TAACACGGCA CTTACACACT |
| 1741 | TGCTGTTCCA CCTTCCCTCA TGCTGTTCCA CCTCCCCTCA GACTATCTTT CAGCCTTCTG |
| 1801 | TCATCAGTAA AATTTATAAA TTTTTTTTAT AACTTCAGTG TAGCTCTCTC CTCTTCAAAT |
| 1861 | AAACATGTCT GCCCTCATGG TTTCG |

The sequence listing of each of SEQ ID NO. 1-10 is also provided in Table 1.

As used herein, the term “KIR typing” refers to the process of determining the genotype of the KIR genes in a subject, including determining the presence and/or identification of one or more specific mutations (e.g., substitution, deletion, or frameshifts) of the KIR genes or alleles in the genome of the subject, and also including determining the presence or absence of one or more specific KIR genes or alleles in the genome of the subject. KIR typing can also include determining the copy number of one or more specific KIRs genes or alleles in the genome of the subject, and their respective mutant forms.

As used herein, the term “carrier” when used in connection with a KIR gene, such as a KIR mutant gene, refers to a subject whose genome includes at least one copy of the gene, and when used in connection with an allele of a gene refers to a subject whose genome includes at least one copy of the specific allele. For example, a carrier of KIR3DL2 refers to a subject whose genome includes at least one copy of KIR3DL2. If a gene has more than one alleles, a carrier of the gene refers to subject whose genome includes at least one copy of at least one allele of the gene.

As used herein, the term “variant allele frequency” or “VAF” refers to the incidence of a gene variant in a population of cells. Alleles are variant forms of a gene that are located at the same position, or genetic locus, on a chromosome. A variant allele frequency is calculated by dividing the number of times the allele of interest is observed in a population of cells by the total number of copies of all the alleles at that particular genetic locus in the population. A variant allele frequency of a particular gene mutation can refer to the amount of DNA present in a sample that contains the mutant allele over the total amount of DNA present in a sample, expressed as a percentage. For example, a VAF % leading to the observed mutation of C336R in KIR3DL2 protein (KIR3DL2 C336R), refers to the amount of DNA present in a sample that contains the mutant allele that leads to the expression of KIR3DL2 C336R mutant protein over the total amount of DNA present in a sample, expressed as a percentage. In some embodiments, the VAF of a particular allel in a sample from the subject may be determined by sequencing, such as by Next Generation Sequencing (NGS), Polymerase Chain Reaction (PCR), DNA microarray, Mass Spectrometry (MS), Single Nucleotide Polymorphism (SNP) assay, denaturing high-performance liquid chromatography (DHPLC), or Restriction Fragment Length Polymorphism (RFLP) assay.

### 2. FTIs, and Compositions Comprising FTIs, for Use in Cancer Treatment

### 2.1. Farnesyltransferase Inhibitors

Provided herein are methods for treating a cancer with a farnesyltransferase inhibitor (FTI) in a selected cancer patient or a selected population of cancer patients. The representative FTIs roughly belong to two classes (Shen et al., Drug Disc. Today 20:2 (2015)). The FTIs in the first class have the basic framework of farnesyldiphosphate (FPP). For instance, FPP analogs with a malonic acid group (Ta) were reported to be FTIs that compete with FPP (Duez, S. et al. Bioorg. Med. Chem. 18:543-556(2010)). In addition, imidazole-containing derivatives linked by an acidic substituent and a peptidyl chain were also synthesized as bisubstrate FTIs, and the designed bisubstrate inhibitors have better affinities than FPP. The FTIs in the second class are peptidomimetic molecules, which can be divided into two groups, namely thiol and non-thiol FTIs. Regarding the thiol FTIs, for instance L-739749, a selective peptidomimetic FTI shows potent antitumor activity in nude mice without system toxicity (Kohl, N. E. et al. PNAS 91:9141-9145(1994)). Additionally, a variety of thiol inhibitors were also developed, such as tripeptidyl FTIs (Lee, H-Y. et al. Bioorg. Med. Chem. Lett. 12:1599-1602(2002)).

For non-thiol FTIs, the heterocycles were therefore widely used to substitute the thiol group to contact with the zinc ion in the binding site. According to the structures of pharmacophoric groups, the nonthiol FTIs can be divided into three classes. The first class is featured by different monocyclic rings, such as L-778123, an FTI in Phase I clinical trials for solid tumors and lymphoma. L-778123 binds into the CAAX peptide site and competes with the CAAX substrate of farnesyltransferase. The second class is represented by tipifarnib in Phase III trials and BMS-214662 in Phase III trials, which are composed of diverse monocyclic rings and bicyclic rings (Harousseau et al. Blood 114:1166-1173 (2009)). The representative inhibitor of the third class is lonafarnib, which is active in Ras-dependent and -independent malignant tumors, and has entered Phase III clinical trials for combating carcinoma, leukemia, and myelodysplastic syndrome. Lonafarnib is an FTI with a tricycle core, which contains a central seven-membered ring fused with two six-membered aromatic rings.

Thus, FTIs as described herein can take on a multitude of forms but share the essential inhibitory function of interfering with or lessening the farnesylation of proteins implicated in cancer and proliferative diseases.

Numerous FTIs are within the scope of the invention and include those described in U.S. Pat. Nos. 5,976,851; 5,972,984; 5,972,966; 5,968,965; 5,968,952; 6,187,786; 6,169,096; 6,037,350; 6,177,432; 5,965,578; 5,965,539; 5,958,939; 5,939,557; 5,936,097; 5,891,889; 5,889,053; 5,880,140; 5,872,135; 5,869,682; 5,861,529; 5,859,015; 5,856,439; 5,856,326; 5,852,010; 5,843,941; 5,807,852; 5,780,492; 5,773,455; 5,767,274; 5,756,528; 5,750,567; 5,721,236; 5,700,806; 5,661,161; 5,602,098; 5,585,359; 5,578,629; 5,534,537; 5,532,359; 5,523,430; 5,504,212; 5,491,164; 5,420,245; and 5,238,922, the disclosures of which are hereby incorporated by reference in their entireties.

FTIs within the scope of the invention also include those described in Thomas et al., Biologics 1: 415-424 (2007); Shen et al., Drug Disc. Today 20:2 (2015); Appels et al., The Oncologist 10:565-578(2005), the disclosures of which are hereby incorporated by reference in their entireties.

In some embodiments, the FTIs include Arglabin (i.e. 1(R)-10-epoxy-5(S),7(S)-guaia-3(4),11(13)-dien-6,12-olide described in WO-98/28303 (NuOncology Labs); perrilyl alcohol described in WO-99/45912 (Wisconsin Genetics); SCH-66336 (lonafarnib), i.e. (+)-(R)-4-[2-[4-(3,10-dibromo-8-chloro-5,6-dihydro-11H-benzo[5,6]cyclohepta[1,2-b]pyridin-11-yl)piperidin-1-yl]-2-oxoethyl]piperidine-1-carboxamide, described in U.S. Pat. No. 5,874,442 (Schering); L778123, i.e. 1-(3-chlorophenyl)-4-[1-(4-cyanobenzyl)-5-imidazolylmethyl]-2-piperazinone, described in WO-00/01691 (Merck); L739749, i.e. compound 2(S)-[2(S)-[2(R)-amino-3-mercapto]propylamino-3(S)-methyl]-pentyloxy-3-phenylpropionyl-methionine sulfone described in WO-94/10138 (Merck); FTI-277, i.e., methyl {N-[2-phenyl-4-N [2(R)-amino-3-mecaptopropylamino] benzoyl]}-methionate (Calbiochem); L744832, i.e, 2S)-2-[[(2S)-2-[(2S,3 S)-2-[(2R)-2-amino-3-mercaptopropyl]amino]-3-methylpentyl]oxy]-1-oxo-3-phenylpropyl]amino]-4-(methylsulfonyl)-butanoic acid 1-methylethyl ester (Biomol International L.P.); CP-609,754 (Pfizer), i.e., (R)-6-[(4-chlorophenyl)-hydroxyl-(1-methyl-1-H-imidazol-5-yl)-methyl]-4-(3-ethynylphenyl)-1-methyl-2-(1H)-quinonlinone and (R)-6-[(4-chlorophenyl)-hydroxyl-(3-methyl-3-H-imidazol-4-yl)-methyl]-4-(3-ethynylphenyl)-1-methyl-2-(1H)-quinolinone; R208176 (Johnson & Johnson), i.e., JNJ-17305457, or (R)-1-(4-chlorophenyl)-1-[5-(3-chlorophenyl)tetrazolo[1,5-a]quinazolin-7-yl]-1-(1-methyl-1H-imidazol-5-yl)methanamine; AZD3409 (AstraZeneca), i.e. (S)-isopropyl 2-(2-(4-fluorophenethyl)-5-((((2S,4S)-4-(nicotinoylthio)pyrrolidin-2-yl)methyl)amino)benzamido)-4-(methylthio)butanoate; BMS 214662 (Bristol-Myers Squibb), i.e. (R)-2,3,4,5-tetrahydro-1-(IH-imidazol-4-ylmethyl)-3-(phenylmethyl)-4-(2-thienylsulphonyl)-1H-1,4-benzodiazapine-7-carbonitrile, described in WO 97/30992 (Bristol Myers Squibb) and Pfizer compounds (A) and (B) described in WO-00/12498 and WO-00/12499.

In some embodiments, the FTI are the non-peptidal, so-called “small molecule” therapeutics, such as are quinolines or quinoline derivatives including:
7-(3-chlorophenyl)-9-[(4-chlorophenyl)-1H-imidazol-1-ylmethyl]-2,3-dihydro-o-1H,5H-benzo[ij]quinolizin-5-one,
7-(3-chlorophenyl)-9-[(4-chlorophenyl)-1H-imidazol-1-ylmethyl]-1,2-dihydro-o-4H-pyrrolo[3,2,1-ij]quinoline-4-one,
8-[amino(4-chlorophenyl)(1-methyl-1H-imidazol-5-yl),methyl]-6-(3-chlorophenyl)-1,2-dihydro-4H-pyrrolo[3,2,1-ij]quinolin-4-one, and
8-[amino(4-chlorophenyl)(1-methyl-1H-imidazol-5-yl)methyl]-6-(3-chlorophenyl)-2,3-dihydro-1H,5H-benzo[ij]quinolizin-5-one.

Tipifarnib is a nonpeptidomimetic FTI (Thomas et al., Biologics 1: 415-424 (2007)). It is a 4,6-disubstituted-1-methylquinolin-2-one derivative ((B)-6-[amino(4-chlorophenyl)(1-methyl-1H-imidazol-5-yl)methyl]-4-(3-chlorophenyl)-1-methyl-2(1H)-quinolinone)) that was obtained by optimization of a quinolone lead identified from compound library screening. Tipifarnib competitively inhibits the CAAX peptide binding site of FTase and is extremely potent and highly selective inhibitor of farnesylation. Tipifarnib is not an inhibitor of geranylgeranyltransferase I. Tipifarnib has manageable safety profile as single agent therapy, is reasonably well tolerated in man and requires twice-daily dosing to obtain effective plasma concentrations.

Tipifarnib is synthesized by the condensation of the anion of 1-methylimidazole with a 6-(4-chlorobenzoyl) quinolone derivative, followed by dehydration. The quinolone intermediate was prepared in four steps by cyclization of N-phenyl-3-(3-chlorophenyl)-2-propenamide, acylation, oxidation and N-methylation. Tipifarnib was identified from Janssen's ketoconazole and retinoic acid catabolism programs as a key structural feature into Ras prenylation process. Tipifarnib is a potent inhibitor of FTase in vitro and is orally active in a variety of animal models. Single agent activity of tipifarnib was observed in unselected tumor populations (AML, MDS/CMML, urothelial cancer, breast cancer, PTCL/CTCL) although a phase III clinic study failed to demonstrate improvement in overall survival.

In some embodiments, provided herein is a method of treating cancer in a subject with an FTI or a pharmaceutical composition having FTI, or selecting a cancer patient for an FTI treatment. The pharmaceutical compositions provided herein contain therapeutically effective amounts of an FTI and a pharmaceutically acceptable carrier, diluent or excipient. In some embodiments, the FTI is tipifarnib; arglabin; perrilyl alcohol; lonafarnib (SCH-66336); L778123; L739749; FTI-277; L744832; R208176; BMS 214662; AZD3409; or CP-609,754. In some embodiments, the FTI is tipifarnib.

### 2.2. FTI Formulations

The FTI can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the FTI is formulated into pharmaceutical compositions using techniques and procedures well known in the art (see, e.g., Ansel Introduction to Pharmaceutical Dosage Forms, Seventh Edition 1999).

In the compositions, effective concentrations of the FTI and pharmaceutically acceptable salts is (are) mixed with a suitable pharmaceutical carrier or vehicle. In some embodiments, the concentrations of the FTI in the compositions are effective for delivery of an amount, upon administration, that treats, prevents, or ameliorates one or more of the symptoms and/or progression of cancer, including haematological cancers and solid tumors.

The compositions can be formulated for single dosage administration. To formulate a composition, the weight fraction of the FTI is dissolved, suspended, dispersed or otherwise mixed in a selected vehicle at an effective concentration such that the treated condition is relieved or ameliorated. Pharmaceutical carriers or vehicles suitable for administration of the FTI provided herein include any such carriers known to those skilled in the art to be suitable for the particular mode of administration.

In addition, the FTI can be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of an FTI provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The FTI is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of FTI in the pharmaceutical composition will depend on absorption, tissue distribution, inactivation and excretion rates of the FTI, the physicochemical characteristics of the FTI, the dosage schedule, and amount administered as well as other factors known to those of skill in the art. For example, the amount that is delivered is sufficient to ameliorate one or more of the symptoms of cancer, including hematopoietic cancers and solid tumors.

In some embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and In some embodiments, from about 10 to about 500 mg of the essential active ingredient or a combination of essential ingredients per dosage unit form.

The FTI may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfate; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the FTI exhibits insufficient solubility, methods for solubilizing compounds can be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, In some embodiments, about 0.1-85% or about 75-95%.

The FTI or pharmaceutically acceptable salts can be prepared with carriers that protect the compound against rapid elimination from the body, such as time release formulations or coatings.

The compositions can include other active compounds to obtain desired combinations of properties. The compounds provided herein, or pharmaceutically acceptable salts thereof as described herein, can also be administered together with another pharmacological agent known in the general art to be of value in treating one or more of the diseases or medical conditions referred to hereinabove, such as diseases related to oxidative stress.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non effervescent or effervescent form with the combination of other ingredients known to those skilled in the art.

In some embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Parenteral administration, generally characterized by injection, either subcutaneously, intramuscularly or intravenously is also provided herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the FTI is adjusted so that an injection provides an effective amount to produce the desired pharmacological effect. The exact dose depends on the age, weight and condition of the patient or animal as is known in the art. The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an FTI is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The FTI can be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Of interest herein are also lyophilized powders, which can be reconstituted for administration as solutions, emulsions and other mixtures. They can also be reconstituted and formulated as solids or gels.

The sterile, lyophilized powder is prepared by dissolving an FTI provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The FTI or pharmaceutical composition having an FTI can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The FTI or pharmaceutical composition having an FTI can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered. These solutions, particularly those intended for ophthalmic use, may be formulated as 0.01%-10% isotonic solutions, pH about 5-7, with appropriate salts.

Other routes of administration, such as transdermal patches, and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams. Tablets and capsules for rectal administration are manufactured using the same pharmaceutically acceptable substance and by the same methods as for formulations for oral administration.

The FTI or pharmaceutical composition having an FTI provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461,6,419,961, 6,589,548, 6,613,358, 6,699,500 and 6,740,634, each of which is incorporated herein by reference. Such dosage forms can be used to provide slow or controlled-release of FTI using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In some embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In some embodiments, the FTI can be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The F can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

The FTI or pharmaceutical composition of FTI can be packaged as articles of manufacture containing packaging material, a compound or pharmaceutically acceptable salt thereof provided herein, which is used for treatment, prevention or amelioration of one or more symptoms or progression of cancer, including hematological cancers and solid tumors, and a label that indicates that the compound or pharmaceutically acceptable salt thereof is used for treatment, prevention or amelioration of one or more symptoms or progression of cancer, including hematological cancers and solid tumors.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

### 2.3. Dosages

In some embodiments, a therapeutically effective amount of the pharmaceutical composition having an FTI is administered orally or parenterally. In some embodiments, the pharmaceutical composition having tipifarnib as the active ingredient and is administered orally in an amount of from 1 up to 1500 mg/kg daily, either as a single dose or subdivided into more than one dose, or more particularly in an amount of from 10 to 1200 mg/kg daily. In some embodiments, the pharmaceutical composition having tipifarnib as the active ingredient and is administered orally in an amount of 100 mg/kg daily, 200 mg/kg daily, 300 mg/kg daily, 400 mg/kg daily, 500 mg/kg daily, 600 mg/kg daily, 700 mg/kg daily, 800 mg/kg daily, 900 mg/kg daily, 1000 mg/kg daily, 1100 mg/kg daily, or 1200 mg/kg daily. In some embodiments, the FTI is tipifarnib.

In some embodiments, the FTI is administered at a dose of 200-1500 mg daily. In some embodiments, the FTI is administered at a dose of 200-1200 mg daily. In some embodiments, the FTI is administered at a dose of 200 mg daily. In some embodiments, the FTI is administered at a dose of 300 mg daily. In some embodiments, the FTI is administered at a dose of 400 mg daily. In some embodiments, the FTI is administered at a dose of 500 mg daily. In some embodiments, the FTI is administered at a dose of 600 mg daily. In some embodiments, the FTI is administered at a dose of 700 mg daily. In some embodiments, the FTI is administered at a dose of 800 mg daily. In some embodiments, the FTI is administered at a dose of 900 mg daily. In some embodiments, the FTI is administered at a dose of 1000 mg daily. In some embodiments, the FTI is administered at a dose of 1100 mg daily. In some embodiments, the FTI is administered at a dose of 1200 mg daily. In some embodiments, the FTI is administered at a dose of 1300 mg daily. In some embodiments, the FTI is administered at a dose of 1400 mg daily. In some embodiments, an FTI is administered at a dose of 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, or 1200 mg daily. In some embodiments, the FTI is tipifarnib.

In some embodiments, the FTI is administered at a dose of 200-1400 mg b.i.d. (i.e., twice a day). In some embodiments, the FTI is administered at a dose of 300-1200 mg b.i.d. In some embodiments, the FTI is administered at a dose of 300-900 mg b.i.d. In some embodiments, the FTI is administered at a dose of 600 mg b.i.d. In some embodiments, the FTI is administered at a dose of 700 mg b.i.d. In some embodiments, the FTI is administered at a dose of 800 mg b.i.d. In some embodiments, the FTI is administered at a dose of 900 mg b.i.d. In some embodiments, the FTI is administered at a dose of 1000 mg b.i.d. In some embodiments, the FTI is administered at a dose of 1100 mg b.i.d. In some embodiments, the FTI is administered at a dose of 1200 mg b.i.d. In some embodiments, an FTI is administered at a dose of 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, or 1200 mg b.i.d. In some embodiments, the FTI is tipifarnib.

As a person of ordinary skill in the art would understand, the dosage varies depending on the dosage form employed, condition and sensitivity of the patient, the route of administration, and other factors. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. During a treatment cycle, the daily dose could be varied. In some embodiments, a starting dosage can be titrated down within a treatment cycle. In some embodiments, a starting dosage can be titrated up within a treatment cycle. The final dosage can depend on the occurrence of dose limiting toxicity and other factors.

In some embodiments, the FTI is administered at a starting dose of 300 mg daily and escalated to a maximum dose of 400 mg, 500 mg, 600 mg, 700 mg, 800 mg, 900 mg, 1000 mg, 1100 mg, or 1200 mg daily. In some embodiments, the FTI is administered at a starting dose of 400 mg daily and escalated to a maximum dose of 500 mg, 600 mg, 700 mg, 800 mg, 900 mg, 1000 mg, 1100 mg, or 1200 mg daily. In some embodiments, the FTI is administered at a starting dose of 500 mg daily and escalated to a maximum dose of 600 mg, 700 mg, 800 mg, 900 mg, 1000 mg, 1100 mg, or 1200 mg daily. In some embodiments, the FTI is administered at a starting dose of 600 mg daily and escalated to a maximum dose of 700 mg, 800 mg, 900 mg, 1000 mg, 1100 mg, or 1200 mg daily. In some embodiments, the FTI is administered at a starting dose of 700 mg daily and escalated to a maximum dose of 800 mg, 900 mg, 1000 mg, 1100 mg, or 1200 mg daily. In some embodiments, the FTI is administered at a starting dose of 800 mg daily and escalated to a maximum dose of 900 mg, 1000 mg, 1100 mg, or 1200 mg daily. In some embodiments, the FTI is administered at a starting dose of 900 mg daily and escalated to a maximum dose of 1000 mg, 1100 mg, or 1200 mg daily. The dose escalation can be done at once, or step wise. For example, a starting dose at 600 mg daily can be escalated to a final dose of 1000 mg daily by increasing by 100 mg per day over the course of 4 days, or by increasing by 200 mg per day over the course of 2 days, or by increasing by 400 mg at once. In some embodiments, the FTI is tipifarnib.

In some embodiments, the FTI is administered at a relatively high starting dose and titrated down to a lower dose depending on the patient response and other factors. In some embodiments, the FTI is administered at a starting dose of 1200 mg daily and reduced to a final dose of 1100 mg, 1000 mg, 900 mg, 800 mg, 700 mg, 600 mg, 500 mg, 400 mg or 300 mg daily. In some embodiments, the FTI is administered at a starting dose of 1100 mg daily and reduced to a final dose of 1000 mg, 900 mg, 800 mg, 700 mg, 600 mg, 500 mg, 400 mg, or 300 mg daily. In some embodiments, the FTI is administered at a starting dose of 1000 mg daily and reduced to a final dose of 900 mg, 800 mg, 700 mg, 600 mg, 500 mg, 400 mg, or 300 mg daily. In some embodiments, the FTI is administered at a starting dose of 900 mg daily and reduced to a final dose of 800 mg, 700 mg, 600 mg, 500 mg, 400 mg, or 300 mg daily. In some embodiments, the FTI is administered at a starting dose of 800 mg daily and reduced to a final dose of 700 mg, 600 mg, 500 mg, 400 mg, or 300 mg daily. In some embodiments, the FTI is administered at a starting dose of 600 mg daily and reduced to a final dose of 500 mg, 400 mg, or 300 mg daily. The dose reduction can be done at once, or step wise. In some embodiments, the FTI is tipifarnib. For example, a starting dose at 900 mg daily can be reduced to a final dose of 600 mg daily by decreasing by 100 mg per day over the course of 3 days, or by decreasing by 300 mg at once.

In some embodiments, the FTI is administered at a starting dose of 300 mg twice a day (b.i.d.) and escalated to a maximum dose of 400 mg, 500 mg, 600 mg, 700 mg, 800 mg, 900 mg, 1000 mg, 1100 mg, or 1200 mg b.i.d. In some embodiments, the FTI is administered at a starting dose of 400 mg b.i.d. and escalated to a maximum dose of 500 mg, 600 mg, 700 mg, 800 mg, 900 mg, 1000 mg, 1100 mg, or 1200 mg b.i.d. In some embodiments, the FTI is administered at a starting dose of 500 mg b.i.d. and escalated to a maximum dose of 600 mg, 700 mg, 800 mg, 900 mg, 1000 mg, 1100 mg, or 1200 mg b.i.d. In some embodiments, the FTI is administered at a starting dose of 600 mg b.i.d. and escalated to a maximum dose of 700 mg, 800 mg, 900 mg, 1000 mg, 1100 mg, or 1200 mg b.i.d. In some embodiments, the FTI is administered at a starting dose of 700 mg b.i.d. and escalated to a maximum dose of 800 mg, 900 mg, 1000 mg, 1100 mg, or 1200 mg b.i.d. In some embodiments, the FTI is administered at a starting dose of 800 mg b.i.d. and escalated to a maximum dose of 900 mg, 1000 mg, 1100 mg, or 1200 mg b.i.d. In some embodiments, the FTI is administered at a starting dose of 900 mg bid and escalated to a maximum dose of 1000 mg, 1100 mg, or 1200 mg b.i.d. The dose escalation can be done at once, or step wise. For example, a starting dose at 600 mg b.i.d. can be escalated to a final dose of 1000 mg b.i.d. by increasing by 100 mg bid over the course of 4 days, or by increasing by 200 mg b.i.d. over the course of 2 days, or by increasing by 400 mg b.i.d. at once. In some embodiments, the FTI is tipifarnib.

In some embodiments, the FTI is administered at a relatively high starting dose and titrated down to a lower dose depending on the patient response and other factors. In some embodiments, the FTI is administered at a starting dose of 1200 mg b.i.d. and reduced to a final dose of 1100 mg, 1000 mg, 900 mg, 800 mg, 700 mg, 600 mg, 500 mg, 400 mg or 300 mg b.i.d. In some embodiments, the FTI is administered at a starting dose of 1100 mg b.i.d. and reduced to a final dose of 1000 mg, 900 mg, 800 mg, 700 mg, 600 mg, 500 mg, 400 mg, or 300 mg b.i.d. In some embodiments, the FTI is administered at a starting dose of 1000 mg b.i.d. and reduced to a final dose of 900 mg, 800 mg, 700 mg, 600 mg, 500 mg, 400 mg, or 300 mg b.i.d. In some embodiments, the FTI is administered at a starting dose of 900 mg b.i.d. and reduced to a final dose of 800 mg, 700 mg, 600 mg, 500 mg, 400 mg, or 300 mg b.i.d. In some embodiments, the FTI is administered at a starting dose of 800 mg b.i.d. and reduced to a final dose of 700 mg, 600 mg, 500 mg, 400 mg, or 300 mg b.i.d. In some embodiments, the FTI is administered at a starting dose of 600 mg b.i.d. and reduced to a final dose of 500 mg, 400 mg, or 300 mg b.i.d. The dose reduction can be done at once, or step wise. In some embodiments, the FTI is tipifarnib. For example, a starting dose at 900 mg b.i.d. can be reduced to a final dose of 600 mg bid by decreasing by 100 mg b.i.d. over the course of 3 days, or by decreasing by 300 mg b.i.d. at once.

A treatment cycle can have different length. In some embodiments, a treatment cycle can be one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In some embodiments, a treatment cycle is 4 weeks. A treatment cycle can have intermittent schedule. In some embodiments, a 2-week treatment cycle can have 5-day dosing followed by 9-day rest. In some embodiments, a 2-week treatment cycle can have 6-day dosing followed by 8-day rest. In some embodiments, a 2-week treatment cycle can have 7-day dosing followed by 7-day rest. In some embodiments, a 2-week treatment cycle can have 8-day dosing followed by 6-day rest. In some embodiments, a 2-week treatment cycle can have 9-day dosing followed by 5-day rest.

In some embodiments, the FTI is administered to a subject on days 1-21 of a 28-day treatment cycle (e.g., twice a day). In some embodiments, the FTI is administered on days 1-7 of a 28-day treatment cycle (e.g., twice a day). In some embodiments, the FTI is administered on days 1-7 and 15-21 of a 28-day treatment cycle (e.g., twice a day). In some embodiments, the FTI is administered for at least 3 cycles or at least 6 cycles (e.g., twice a day). In some of these embodiments, the FTI is tipifarnib, and the dose of tipifarnib is from 200 mg to 900 mg twice a day (e.g. 200 mg, 300 mg, 400 mg, 500 mg, 600 mg, 700 mg, 800 mg, or 900 mg). In some of these embodiments, the FTI is tipifarnib, and the dose of tipifarnib is from 250 mg to 1000 mg twice a day (e.g. 250 mg, 350 mg, 450 mg, 550 mg, 650 mg, 750 mg, 850 mg, 950 mg, or 1000 mg). In some embodiments, the FTI is administered to a subject for at least or more than 3 months, 4 months, 5 months, 6 months, 7 months, 8 months, 9 months, 1 year, 15 months, 1.5 years, 18 months, 2 years or 3 years. In some embodiments, the FTI is administered to a subject for at least or more than 3 months. In some embodiments, the FTI is administered to a subject for at least or more than 6 months. In some embodiments, the FTI is administered to a subject for at least or more than 1 year. In some embodiments, the subject remains responsive to treatment with an FTI for at least or more than 3 months, 4 months, 5 months, 6 months, 7 months, 8 months, 9 months, 1 year, 15 months, 1.5 years, 18 months, 2 years or 3 years. In some embodiments, the subject remains responsive to treatment with an FTI for at least or more than 3 months. In some embodiments, the subject remains responsive to treatment with an FTI for at least or more than 6 months. In some embodiments, the subject remains responsive to treatment with an FTI for at least or more than 1 year.

In some embodiments, the FTI is administered daily for 3 out of 4 weeks in repeated 4 week cycles. In some embodiments, the FTI is administered daily in alternate weeks (one week on, one week off) in repeated 4 week cycles. In some embodiments, the FTI is administered at a dose of 300 mg b.i.d. orally for 3 out of 4 weeks in repeated 4 week cycles. In some embodiments, the FTI is administered at a dose of 600 mg b.i.d. orally for 3 out of 4 weeks in repeated 4 week cycles. In some embodiments, the FTI is administered at a dose of 900 mg b.i.d. orally in alternate weeks (one week on, one week off) in repeated 4 week cycles. In some embodiments, the FTI is administered at a dose of 1200 mg b.i.d. orally in alternate weeks (days 1-7 and 15-21 of repeated 28-day cycles). In some embodiments, the FTI is administered at a dose of 1200 mg b.i.d. orally for days 1-5 and 15-19 out of repeated 28-day cycles.

In some embodiments, a 900 mg b.i.d. tipifarnib alternate week regimen can be used. Under the regimen, patients receive a starting dose of 900 mg, po, b.i.d. on days 1-7 and 15-21 of 28-day treatment cycles. In some embodiments, patients receive two treatment cycles. In some embodiments, patients receive three treatment cycles. In some embodiments, patients receive four treatment cycles. In some embodiments, patients receive five treatment cycles. In some embodiments, patients receive six treatment cycles. In some embodiments, patients receive seven treatment cycles. In some embodiments, patients receive eight treatment cycles. In some embodiments, patients receive nine treatment cycles. In some embodiments, patients receive ten treatment cycles. In some embodiments, patients receive eleven treatment cycles. In some embodiments, patients receive twelve treatment cycles. In some embodiments, patients receive more than twelve treatment cycles.

In the absence of unmanageable toxicities, subjects can continue to receive the tipifarnib treatment for up to 12 months. The dose can also be increased to 1200 mg b.i.d. if the subject is tolerating the treatment well. Stepwise 300 mg dose reductions to control treatment-related, treatment-emergent toxicities can also be included.

In some other embodiments, tipifarnib is given orally at a dose of 300 mg b.i.d. daily for 21 days, followed by 1 week of rest, in 28-day treatment cycles (21-day schedule; Cheng D T, et al., J Mol Diagn. (2015) 17(3):251-64). In some embodiments, a 5-day dosing ranging from 25 to 1300 mg b.i.d. followed by 9-day rest is adopted (5-day schedule; Zujewski J., J Clin Oncol., (2000) February; 18(4):927-41). In some embodiments, a 7-day b.i.d. dosing followed by 7-day rest is adopted (7-day schedule; Lara P N Jr., Anticancer Drugs., (2005) 16(3):317-21; Kirschbaum M R, Leukemia., (2011) October; 25(10):1543-7). In the 7-day schedule, the patients can receive a starting dose of 300 mg b.i.d. with 300 mg dose escalations to a maximum planned dose of 1800 mg b.i.d. In the 7-day schedule study, patients can also receive tipifarnib b.i.d. on days 1-7 and days 15-21 of 28-day cycles at doses up to 1600 mg b.i.d.

In previous studies FTI were shown to inhibit the growth of mammalian tumors when administered as a twice daily dosing schedule. It was found that administration of an FTI in a single dose daily for one to five days produced a marked suppression of tumor growth lasting out to at least 21 days. In some embodiments, FTI is administered at a dosage range of 50-400 mg/kg. In some embodiments, FTI is administered at 200 mg/kg. Dosing regimen for specific FTIs are also well known in the art (e.g., U.S. Pat. No. 6,838,467, which is incorporated herein by reference in its entirety). For example, suitable dosages for the compounds Arglabin (WO98/28303), perrilyl alcohol (WO 99/45712), SCH-66336 (U.S. Pat. No. 5,874,442), L778123 (WO 00/01691), 2(S)-[2(S)-[2(R)-amino-3-mercapto]propylamino-3(S)-methyl]-pentyloxy-3-phenylpropionyl-methionine sulfone (WO94/10138), BMS 214662 (WO 97/30992), AZD3409; Pfizer compounds A and B (WO 00/12499 and WO 00/12498) are given in the aforementioned patent specifications which are incorporated herein by reference or are known to or can be readily determined by a person skilled in the art.

In relation to perrilyl alcohol, the medicament may be administered 1-4 g per day per 150 lb human patient. Preferably, 1-2 g per day per 150 lb human patient. SCH-66336 typically can be administered in a unit dose of about 0.1 mg to 100 mg, more preferably from about 1 mg to 300 mg according to the particular application. Compounds L778123 and 1-(3-chlorophenyl)-4-[1-(4-cyanobenzyl)-5-imidazolylmethyl]-2-piperazinone may be administered to a human patient in an amount between about 0.1 mg/kg of body weight to about 20 mg/kg of body weight per day, preferably between 0.5 mg/kg of bodyweight to about 10 mg/kg of body weight per day.

Pfizer compounds A and B may be administered in dosages ranging from about 1.0 mg up to about 500 mg per day, preferably from about 1 to about 100 mg per day in single or divided (i.e. multiple) doses. Therapeutic compounds will ordinarily be administered in daily dosages ranging from about 0.01 to about 10 mg per kg body weight per day, in single or divided doses. BMS 214662 may be administered in a dosage range of about 0.05 to 200 mg/kg/day, preferably less than 100 mg/kg/day in a single dose or in 2 to 4 divided doses.

### 2.4. Combination Therapies

In some embodiments, the FTI treatment is administered in combination with radiotherapy, or radiation therapy. Radiotherapy includes using γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287; all of which are hereby incorporated by references in their entireties), and UV-irradiation. It is most likely that all of these factors affect a broad range of damage on DNA, on the precursors of DNA, on the replication and repair of DNA, and on the assembly and maintenance of chromosomes.

In some embodiments, a therapeutically effective amount of the pharmaceutical composition having an FTI is administered that effectively sensitizes a tumor in a host to irradiation. (U.S. Pat. No. 6,545,020, which is hereby incorporated by reference in its entirety). Irradiation can be ionizing radiation and in particular gamma radiation. In some embodiments, the gamma radiation is emitted by linear accelerators or by radionuclides. The irradiation of the tumor by radionuclides can be external or internal.

Irradiation can also be X-ray radiation. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the administration of the pharmaceutical composition commences up to one month, in particular up to 10 days or a week, before the irradiation of the tumor. Additionally, irradiation of the tumor is fractionated the administration of the pharmaceutical composition is maintained in the interval between the first and the last irradiation session.

The amount of FTI, the dose of irradiation and the intermittence of the irradiation doses will depend on a series of parameters such as the type of tumor, its location, the patients' reaction to chemo- or radiotherapy and ultimately is for the physician and radiologists to determine in each individual case.

In some embodiments, the methods provided herein further include administering a therapeutically effective amount of a second active agent or a support care therapy. The second active agent can be a chemotherapeutic agent. A chemotherapeutic agent or drug can be categorized by its mode of activity within a cell, for example, whether and at what stage they affect the cell cycle. Alternatively, an agent can be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabine, navelbine, transplatinum, and pharmaceutically acceptable salts, acids, or derivatives of any of the above.

The second active agents can be large molecules (e.g., proteins) or small molecules (e.g., synthetic inorganic, organometallic, or organic molecules). In some embodiments, the second active agent is a DNA-hypomethylating agent, a therapeutic antibody that specifically binds to a cancer antigen, a hematopoietic growth factor, cytokine, anti-cancer agent, antibiotic, cox-2 inhibitor, immunomodulatory agent, anti-thymocyte globulin, immunosuppressive agent, corticosteroid or a pharmacologically active mutant or derivative thereof.

In some embodiments, the second active agent is a DNA hypomethylating agent, such as a cytidine analog (e.g., azacitidine) or a 5-azadeoxycytidine (e.g. decitabine). In some embodiments, the second active agent is a cytoreductive agent, including but not limited to Induction, Topotecan, Hydrea, PO Etoposide, Lenalidomide, LDAC, and Thioguanine. In some embodiments, the second active agent is Mitoxantrone, Etoposide, Cytarabine, or Valspodar. In some embodiment, the second active agent is Mitoxantrone plus Valspodar, Etoposide plus Valspodar, or Cytarabine plus Valspodar. In some embodiment, the second active agent is idarubicin, fludarabine, topotecan, or ara-C. In some other embodiments, the second active agent is idarubicin plus ara-C, fludarabine plus ara-C, mitoxantrone plus ara-C, or topotecan plus ara-C. In some embodiments, the second active agent is a quinine. In some embodiments, the second active agent is dasatinib or imatinib. Other combinations of the agents specified above can be used, and the dosages can be determined by the physician.

For any specific cancer type described herein, treatments as described herein or otherwise available in the art can be used in combination with the FTI treatment. For example, drugs that can be used in combination with the FTI include belinostat (Beleodaq®) and pralatrexate (Folotyn®), marketed by Spectrum Pharmaceuticals, romidepsin (Istodax®), marketed by Celgene, and brentuximab vedotin (Adcetris®) (for ALCL), marketed by Seattle Genetics; drugs that can be used in combination with the FTI include azacytidine (Vidaza®) and lenalidomide (Revlimid®), marketed by Celgene, and decitabine (Dacogen®) marketed by Otsuka and Johnson & Johnson; drugs that can be used in combination with the FTI for thyroid cancer include AstraZeneca's vandetanib (Caprelsa), Bayer's sorafenib (Nexavar), Exelixis' cabozantinib (Cometriq®) and Eisai's lenvatinib (Lenvima®).

Non-cytotoxic therapies such as tpralatrexate (Folotyn®), romidepsin (Istodax®) and belinostat (Beleodaq®) can also be used in combination with the FTI treatment.

In some embodiments, the second active agent is an immunotherapy agent. In some embodiments, the second active agent is anti-PD1 antibody or anti-PDL1 antibody.

In some embodiments, it is contemplated that the second active agent or second therapy used in combination with an FTI can be administered before, at the same time, or after the FTI treatment. In some embodiments, the second active agent or second therapy used in combination with an FTI can be administered before the FTI treatment. In some embodiments, the second active agent or second therapy used in combination with an FTI can be administered at the same time as FTI treatment. In some embodiments, the second active agent or second therapy used in combination with an FTI can be administered after the FTI treatment.

The FTI treatment can also be administered in combination with a bone marrow transplant. In some embodiments, the FTI is administered before the bone marrow transplant. In other embodiments, the FTI is administered after the bone marrow transplant.

A person of ordinary skill in the art would understand that the methods described herein include using any permutation or combination of the specific FTI, formulation, dosing regimen, additional therapy to treat a subject described herein.

### 3. Treatment of Cancer Based on the Mutation Status of MR

Provided herein are methods of selection of cancer patients for treatment with an FTI which are based, in part, on the discovery that the mutation status in a member of the KIR family is associated with clinical benefits of FTI and can be used to predict the responsiveness of a cancer patient to an FTI treatment. Accordingly, provided herein are methods for predicting responsiveness of a cancer patient to an FTI treatment, methods for cancer patient population selection for an FTI treatment, and methods for treating cancer in a subject with a therapeutically effective amount of an FTI, based on the mutation status of a member of the KIR family (e.g., KIR2DL1, KIR2DL3, KIR2DL4, KIR3DL1, and/or KIR3DL2) in a sample from the patient. In particular, provided herein are methods for treating a KIR-mutant cancer, i.e., a cancer known to have or determined to have a mutation in a member of the KIR family. Also provided herein are methods for treating patients having a cancer and a mutation in a member of the KIR family (such as a mutation in a member of the KIR family in a tumor cell or tissue). Provided herein are also methods for treating a premalignant condition in a subject with an FTI, and methods for selecting patients with a premalignant condition for an FTI treatment based on the mutation status of a member of the KIR family (e.g., KIR2DL1, KIR2DL3, KIR2DL4, KIR3DL1, and/or KIR3DL2). In some embodiments, the method includes predicting the responsiveness of a subject having cancer for an FTI treatment, selecting a cancer patient for an FTI treatment, stratifying cancer patients for an FTI treatment, and/or increasing the responsiveness of a cancer patient population for an FTI treatment based on identification of specific KIR family member(s) mutations. In some embodiments, the methods include analyzing a sample from the subject having cancer to determining that the subject has KIR-mutant cancer prior to administering the FTI to the subject. In some embodiments, the method further includes determining a KIR-mutant cancer variant allele frequency (VAF) in a sample from the cancer subject, wherein the KIR-mutant cancer is selected from the group consisting of: a KIR2DL1-mutant, a KIR2DL3-mutant, a KIR2DL4-mutant, a KIR3DL1-mutant, and/or a KIR3DL2-mutant. In some embodiments, the method further provides determining the VAF of a mutation of a member of the KIR family (e.g., KIR2DL1, KIR2DL3, KIR2DL4, KIR3DL1, and/or KIR3DL2) from the sample from the cancer subject. In some embodiments, the method further provides determining the VAF of a KIR3DL2 mutation from the sample from the cancer subject. In some embodiments, the method further provides determining the VAF of the KIR3DL2 mutation selected from the group consisting of: a KIR3DL2 C336R mutation, a KIR3DL2 Q386E mutation, or a KIR3DL2 C336R/Q386E mutation, from the sample from the cancer subject. In some embodiments, the FTI is tipifarnib. In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, provided herein are methods for predicting responsiveness of a MDS patient to an FTI treatment, methods for MDS patient population selection for an FTI treatment, and methods for treating MDS in a subject with a therapeutically effective amount of an FTI, based on the mutation status of a member of the KIR family (such as KIR2DL1, KIR2DL3, KIR2DL4, KIR3DL1, and/or KIR3DL2) in a sample from the patient (e.g., tumor sample). In some embodiments, provided herein are methods for predicting responsiveness of a MPN patient to an FTI treatment, methods for MPN patient population selection for an FTI treatment, and methods for treating MPN in a subject with a therapeutically effective amount of an FTI, based on the mutation status of a member of the KIR family (such as KIR2DL1, KIR2DL3, KIR2DL4, KIR3DL1, and/or KIR3DL2) in a sample from the patient (e.g., tumor sample). In some embodiments, provided herein are methods for predicting responsiveness of an AML patient to an FTI treatment, methods for AML patient population selection for an FTI treatment, and methods for treating AML in a subject with a therapeutically effective amount of an FTI, based on the mutation status of a member of the KIR family (such as KIR2DL1, KIR2DL3, KIR2DL4, KIR3DL1, and/or KIR3DL2) in a sample from the patient (e.g., tumor sample). In some embodiments, provided herein are methods for predicting responsiveness of a JMML patient to an FTI treatment, methods for JMML patient population selection for an FTI treatment, and methods for treating JMML in a subject with a therapeutically effective amount of an FTI, based on the mutation status of a member of the KIR family (such as KIR2DL1, KIR2DL3, KIR2DL4, KIR3DL1, and/or KIR3DL2) in a sample from the patient (e.g., tumor sample).

### 3.1. KIR Mutation Status

In some embodiments, the cancer to be treated by methods provided herein can have a KIR mutation or mutations (e.g., one or more mutations in a member of the KIR family such as KIR2DL1, KIR2DL3, KIR2DL4, KIR3DL1, and/or KIR3DL2). In some embodiments, mutation status of a gene of the KIR family can be determined in the form of a companion diagnostic to the FTI treatment, such as the tipifarnib treatment. The companion diagnostic can be performed at the clinic site where the patient receives the tipifarnib treatment, or at a separate site. Methods provided herein or otherwise known in the art can be used to determine the mutation status of a member of the KIR family (e.g., KIR2DL1, KIR2DL3, KIR2DL4, KIR3DL1, and/or KIR3DL2). In some embodiments, the mutation status of a gene of the KIR family (e.g., KIR2DL1, KIR2DL3, KIR2DL4, KIR3DL1, and/or KIR3DL2) can be determined by a next generation sequencing (NGS)-based assay. In some embodiments, the mutation status of a gene of the KIR family (e.g., KIR2DL1, KIR2DL3, KIR2DL4, KIR3DL1, and/or KIR3DL2) can be determined by a qualitative PCR-based assay.

Provided herein are methods of selection of cancer patients for treatment with an FTI based on the presence of a mutation in a member of the KIR family (e.g., KIR2DL1, KIR2DL3, KIR2DL4, KIR3DL1, and/or KIR3DL2). In some embodiments, provided herein is a method of treating a cancer in a subject based on the presence of a mutation in a member of the KIR family (e.g., KIR2DL1, KIR2DL3, KIR2DL4, KIR3DL1, and/or KIR3DL2). The method provided herein includes (a) determining the presence or absence of a mutation in a member of the KIR family (e.g., KIR2DL1, KIR2DL3, KIR2DL4, KIR3DL1, and/or KIR3DL2) in a sample from the subject, and subsequently (b) administering a therapeutically effective amount of an FTI to the subject if the sample is determined to have a mutation in a member of the KIR family. The sample can be a tumor sample, a bone marrow sample or a plasma sample. In some embodiments, the methods include (a) determining a cancer patient to have a mutation in a member of the KIR family (e.g., KIR2DL1, KIR2DL3, KIR2DL4, KIR3DL1, and/or KIR3DL2), and subsequently (b) administering a therapeutically effective amount of an FTI to the subject.

In some embodiments, the method of treating a cancer in a subject in need thereof, comprises administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2. In some embodiments, the method includes predicting the responsiveness of a subject having cancer for an FTI treatment, selecting a cancer patient for an FTI treatment, stratifying cancer patients for an FTI treatment, and/or increasing the responsiveness of a cancer patient population for an FTI treatment based on identification of specific KIR family member(s) mutations. In some embodiments, the methods include analyzing a sample from the subject having cancer to determining that the subject has KIR-mutant cancer prior to administering the FTI to the subject. In some embodiments, the method further provides determining the VAF of a mutation of the KIR family member (e.g., KIR2DL1, KIR2DL3, KIR2DL4, KIR3DL1, and/or KIR3DL2) from the sample from the cancer subject. In some embodiments, the FTI is tipifarnib. In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, the method of treating a cancer in a subject in need thereof, comprises administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, and wherein the KIR2DL1, KIR2DL3, KIR2DL4, KIR3DL1, and/or KIR3DL2, has two of more mutations comprising two or more modifications at two or more codons that endode two or more amino acids in the extracellular domain, at two or more codons that endode two or more amino acids in the cytoplasmic domain, or combinations thereof.

In some embodiments, the method of treating a cancer in a subject in need thereof, comprises administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, and wherein the KIR2DL1, KIR2DL3, KIR2DL4, KIR3DL1, and/or KIR3DL2, has three of more mutations comprising three or more modifications at three or more codons that endode three or more amino acids in the extracellular domain, at three or more codons that endode three or more amino acids in the cytoplasmic domain, or combinations thereof.

In some embodiments, the method of treating a cancer in a subject in need thereof, comprises administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, and wherein the KIR2DL1, KIR2DL3, KIR2DL4, KIR3DL1, and/or KIR3DL2, has four of more mutations comprising four or more modifications at four or more codons that endode four or more amino acids in the extracellular domain, at four or more codons that endode four or more amino acids in the cytoplasmic domain, or combinations thereof.

In some embodiments, the method of treating a cancer in a subject in need thereof, comprises administering a therapeutically effective amount of an FTI to said subject, wherein the cancer is a cancer known to have or determined to have a mutation in a member of the KIR family selected from the group consisting of: KIR2DL1, KIR2DL3, KIR2DL4, KIR3DL1, and KIR3DL2, and wherein the KIR-mutant cancer is a cancer known to have or determined to have a mutation in two, three, four, or each of the members of the KIR family selected from the group consisting of KIR2DL1, KIR2DL3, KIR2DL4, KIR3DL1, and KIR3DL2.

In some embodiments, provided herein are methods for treating cancer in a subject by administering a therapeutically effective amount of an FTI to the subject, wherein the subject (e.g., a human) is a carrier of a mutation in a member of the KIR family selected from the group consisting of: KIR2DL1, KIR2DL3, KIR2DL4, KIR3DL1, and KIR3DL2. In some embodiments, provided herein is a method for treating cancer in a subject by KIR typing the subject, and administering a therapeutically effective amount of an FTI to the subject, wherein the subject is a carrier of a KIR mutation (e.g., a mutation at amino acid) in a KIR family selected from the group consisting of: KIR2DL1, KIR2DL3, KIR2DL4, KIR3DL1, and KIR3DL2.

In some embodiments, the method of treating a cancer in a subject in need thereof, comprises administering a therapeutically effective amount of an FTI, optionally tipifarnib, to said subject, wherein the cancer is a cancer known to have or determined to have a mutation in KIR2DL1, such as two, three, four, or more mutations, in KIR2DL1. In some embodiments, the methods provided herein include determining the presence of the mutation in KIR2DL1 (e.g., determining the presence of the two, three, four, or more mutations, in KIR2DL1) in a sample from a subject having cancer, and administering a therapeutically effective amount of an FTI to said subject if the mutation in KIR2DL1 is present (e.g., if the two, three, four, or more mutations, in KIR2DL1 are present). In some embodiments, the mutation is or comprises a modification in a codon of KIR2DL1 encoding an amino acid in the extracellular domain, in the cytoplasmic domain, or combinations thereof. In some embodiments, the methods provided herein include determining the presence of has two, three, four, or more, mutations in the KIR2DL1 comprising two, three, four, or more, modifications at two, three, four, or more, codons that endode two, three, four, or more, amino acids in the extracellular domain, at two, three, four, or more, codons that endode two, three, four, or more, amino acids in the cytoplasmic domain, or combinations thereof. In some embodiments, the mutation is or comprises a modification in a codon of KIR2DL1 encoding an amino acid in the extracellular domain selected from a group consisting of: M65, H77, A83, S88, T91, L140, N178, G179, D184, R197, F202, and H203. In some embodiments, the mutation in the extracellular domain of KIR2DL1 is selected from a group consisting of: M65T, H77N, H77L, A83G, S88G, T91K, L140Q, N178D, G179R, D184N, R197T, F202L, and H203R. In some embodiments, the mutation is or comprises a modification in a codon of KIR2DL1 encoding an amino acid in the extracellular D2 domain selected from a group consisting of: N178, G179, D184, R197, F202, and H203. In some embodiments, the mutation in the extracellular D2 domain of KIR2DL1 is selected from a group consisting of: N178D, G179R, D184N, R197T, F202L, and H203R. In some embodiments, the mutation is or comprises a modification in a codon of KIR2DL1 encoding an amino acid in the extracellular D2 domain selected from a group consisting of: N178, G179, D184, R197, and F202. In some embodiments, the mutation results in a change in amino acid in KIR2DL1 (SEQ ID NO.: 1) selected from a group consisting of: M65T, H77N, H77L, A83G, S88G, T91K, L140Q, N178D, G179R, D184N, R197T, F202L, and H203R. In some embodiments, the mutation is or comprises a modification in a codon of KIR2DL1 encoding the amino acid M65. In some embodiments, the mutation is or comprises a modification in a codon of KIR2DL1 encoding the amino acid H77. In some embodiments, the mutation is or comprises a modification in a codon of KIR2DL1 encoding the amino acid A83. In some embodiments, the mutation is or comprises a modification in a codon of KIR2DL1 encoding the amino acid S88. In some embodiments, the mutation is or comprises a modification in a codon of KIR2DL1 encoding the amino acid T91. In some embodiments, the mutation is or comprises a modification in a codon of KIR2DL1 encoding the amino acid L140. In some embodiments, the mutation is or comprises a modification in a codon of KIR2DL1 encoding the amino acid N178. In some embodiments, the mutation is or comprises a modification in a codon of KIR2DL1 encoding the amino acid G179. In some embodiments, the mutation is or comprises a modification in a codon of KIR2DL1 encoding the amino acid D184. In some embodiments, the mutation is or comprises a modification in a codon of KIR2DL1 encoding the amino acid R197. In some embodiments, the mutation is or comprises a modification in a codon of KIR2DL1 encoding the amino acid F202. In some embodiments, the mutation is or comprises a modification in a codon of KIR2DL1 encoding the amino acid H203. In some embodiments, the mutation results in a change (e.g., a substitution or deletion) in amino acid N178 (e.g., N178D mutation) of KIR2DL1. In some embodiments, the mutation in KIR2DL1 is M65T. In some embodiments, the mutation in KIR2DL1 is H77N. In some embodiments, the mutation in KIR2DL1 is H77L. In some embodiments, the mutation in KIR2DL1 is A83G. In some embodiments, the mutation in KIR2DL1 is S88G. In some embodiments, the mutation in KIR2DL1 is T91K. In some embodiments, the mutation in KIR2DL1 is L140Q. In some embodiments, the mutation in KIR2DL1 is N178D. In some embodiments, the mutation in KIR2DL1 is G179R. In some embodiments, the mutation in KIR2DL1 is D184N. In some embodiments, the mutation in KIR2DL1 is R197T. In some embodiments, the mutation in KIR2DL1 is F202L. In some embodiments, the mutation in KIR2DL1 is H203R. In some embodiments, the mutation in the extracellular D2 domain of KIR2DL1 is selected from a group consisting of: N178D, G179R, D184N, R197T, and F202L. In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, the method of treating a cancer in a subject in need thereof, comprises administering a therapeutically effective amount of an FTI, optionally tipifarnib, to said subject, wherein the cancer is a cancer known to have or determined to have a mutation in KIR2DL3, such as two, three, four, or more mutations, in KIR2DL3. In some embodiments, the methods provided herein include determining the presence of the mutation in KIR2DL3 (e.g., determining the presence of the two, three, four, or more mutations, in KIR2DL3) in a sample from a subject having cancer, and administering a therapeutically effective amount of an FTI to said subject if the mutation in KIR2DL3 is present (e.g., if the two, three, four, or more mutations, in KIR2DL3 are present). In some embodiments, the mutation is or comprises a modification in a codon of KIR2DL3 encoding an amino acid in the extracellular domain, in the cytoplasmic domain, or combinations thereof. In some embodiments, the methods provided herein include determining the presence of has two, three, four, or more, mutations in the KIR2DL3 comprising two, three, four, or more, modifications at two, three, four, or more, codons that endode two, three, four, or more, amino acids in the extracellular domain, at two, three, four, or more, codons that endode two, three, four, or more, amino acids in the cytoplasmic domain, or combinations thereof. In some embodiments, the mutation is or comprises a modification in a codon of KIR2DL3 encoding an amino acid selected from a group consisting of: F66, R162, R169, F171, S172, E295, R318, I330, I331, and V332. In some embodiments, the mutation in KIR2DL3 is selected from a group consisting of: F66Y, R162T, R169C, F171L, S172P, E295D, R318C, I330T, I331T, and V332M. In some embodiments, the mutation is or comprises a modification in a codon of KIR2DL3 encoding the amino acid R162 and/or E295. In some embodiments, the mutation in KIR2DL3 is or comprises the R162T and/or the E295D. In some embodiments, the mutation is or comprises a modification in a codon of KIR2DL3 encoding an amino acid in the extracellular D2 domain selected from a group consisting of: F66, R162, R169, F171, and S172. In some embodiments, the mutation in the extracellular D2 domain of KIR2DL3 is selected from a group consisting of: F66Y, R162T, R169C, F171L, and S172P. In some embodiments, the mutation in KIR2DL3 in the extracellular D2 domain is or comprises an amino acid modification at the codon R162. In some embodiments, the mutation in the extracellular D2 domain of KIR2DL3 is R162T. In some embodiments, the mutation is or comprises a modification in a codon of KIR2DL3 encoding an amino acid in the cytoplasmic domain selected from a group consisting of: E295, R318, I330, I331, and V332. In some embodiments, the mutation in the cytoplasmic domain of KIR2DL3 is selected from a group consisting of: E295D, R318C, I330T, I331T, and V332M. In some embodiments, the mutation in the cytoplasmic domain of KIR2DL3 is within or near the CK2 site, the PKC site, and/or the immunoreceptor tyrosine-based inhibitory motif 2 (ITIM 2), of said cytoplasmic domain. In some embodiments, the mutation is or comprises a modification in a codon of KIR2DL3 encoding the amino acid within or near the CK2 site of the cytoplasmic domain, such as E295. In some embodiments, the mutation within or near the CK2 site of the cytoplasmic domain of KIR2DL3 is E295D. In some embodiments, the mutation is or comprises a modification in a codon of KIR2DL3 encoding the amino acid within or near the PKC site of the cytoplasmic domain, such as R318. In some embodiments, the mutation within or near the PKC site of the cytoplasmic domain of KIR2DL3 is R318C. In some embodiments, the mutation is or comprises a modification in a codon of KIR2DL3 encoding the amino acids within or near the ITIM 2 of the cytoplasmic domain selected from a group consisting of: I330, I331, and V332. In some embodiments, the mutation within or near the ITIM 2 of the cytoplasmic domain of KIR2DL3 is selected from a group consisting of: I330T, I331T, and V332M. In some embodiments, the mutation results in a change in amino acid in KIR2DL3 (SEQ ID NO.: 3) selected from a group consisting of: F66, R162, R169, F171, S172, E295, R318, I330, I331, and V332. In some embodiments, the mutation is or comprises a modification in a codon of KIR2DL3 encoding the amino acid F66. In some embodiments, the mutation is or comprises a modification in a codon of KIR2DL3 encoding the amino acid R162. In some embodiments, the mutation is or comprises a modification in a codon of KIR2DL3 encoding the amino acid R169. In some embodiments, the mutation is or comprises a modification in a codon of KIR2DL3 encoding the amino acid F171. In some embodiments, the mutation is or comprises a modification in a codon of KIR2DL3 encoding the amino acid S172. In some embodiments, the mutation is or comprises a modification in a codon of KIR2DL3 encoding the amino acid E295. In some embodiments, the mutation is or comprises a modification in a codon of KIR2DL3 encoding the amino acid R318. In some embodiments, the mutation is or comprises a modification in a codon of KIR2DL3 encoding the amino acid I330. In some embodiments, the mutation is or comprises a modification in a codon of KIR2DL3 encoding the amino acid I331. In some embodiments, the mutation is or comprises a modification in a codon of KIR2DL3 encoding the amino acid V332. In some embodiments, the mutation results in a change (e.g., a substitution or deletion) in amino acid E295 (e.g., E295D mutation) of KIR2DL3. In some embodiments, the mutation in KIR2DL3 is F66Y. In some embodiments, the mutation in KIR2DL3 is R162T. In some embodiments, the mutation in KIR2DL3 is R169C. In some embodiments, the mutation in KIR2DL3 is F171L. In some embodiments, the mutation in KIR2DL3 is S172P. In some embodiments, the mutation in KIR2DL3 is E295D. In some embodiments, the mutation in KIR2DL3 is R318C. In some embodiments, the mutation in KIR2DL3 is I330T. In some embodiments, the mutation in KIR2DL3 is I331T. In some embodiments, the mutation in KIR2DL3 is V332M. In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, the method of treating a cancer in a subject in need thereof, comprises administering a therapeutically effective amount of an FTI, optionally tipifarnib, to said subject, wherein the cancer is a cancer known to have or determined to have a mutation in KIR2DL4, such as two, three, four, or more mutations, in KIR2DL4. In some embodiments, the methods provided herein include determining the presence of the mutation in KIR2DL4 (e.g., determining the presence of the two, three, four, or more mutations, in KIR2DL4) in a sample from a subject having cancer, and administering a therapeutically effective amount of an FTI to said subject if the mutation in KIR2DL4 is present (e.g., if the two, three, four, or more mutations, in KIR2DL4 are present). In some embodiments, the mutation is or comprises a modification in a codon of KIR2DL4 encoding an amino acid in the extracellular domain, in the cytoplasmic domain, or combinations thereof. In some embodiments, the methods provided herein include determining the presence of has two, three, four, or more, mutations in the KIR2DL4 comprising two, three, four, or more, modifications at two, three, four, or more, codons that endode two, three, four, or more, amino acids in the extracellular domain, at two, three, four, or more, codons that endode two, three, four, or more, amino acids in the cytoplasmic domain, or combinations thereof. In some embodiments, the mutation is or comprises a modification in a codon of KIR2DL4 encoding an amino acid selected from a group consisting of: R50, H52, R55, N58, T61, K65, Q149, I154, E162, L166, I174, A238, and S267. In some embodiments, the mutation in KIR2DL4 is selected from a group consisting of: R50L, H52R, R55L, N58T, T61R, K65E, Q149K, Q149R, I154M, E162K, E162G, L166P, I174V, A238P, and S267fs. In some embodiments, the mutation is or comprises a modification in a codon of KIR2DL4 encoding an amino acid in the extracellular domain selected from a group consisting of: R50, H52, R55, N58, T61, K65, Q149, I154, E162, L166, and I174. In some embodiments, the mutation in the extracellular domain of KIR2DL4 is selected from a group consisting of: R50L, H52R, R55L, N58T, T61R, K65E, Q149K, Q149R, I154M, E162K, E162G, L166P, and I174V. In some embodiments, the mutation is or comprises a modification in a codon of KIR2DL4 encoding an amino acid in the extracellular D2 domain selected from a group consisting of: Q149, I154, E162, L166, and I174. In some embodiments, the mutation in the extracellular D2 domain of KIR2DL4 is selected from a group consisting of: Q149K, Q149R, I154M, E162K, E162G, L166P, and I174V. In some embodiments, the mutation is or comprises a modification in a codon of KIR2DL4 encoding the amino acid Q149 and/or I154 in the extracellular D2 domain. In some embodiments, the mutation in the extracellular D2 domain of KIR2DL4 is or comprises the Q149K, Q149R, and/or I154M. In some embodiments, the mutation is or comprises a modification in a codon of KIR2DL4 encoding an amino acid in the cytoplasmic domain selected from a group consisting of: A238 and S267. In some embodiments, the mutation in the cytoplasmic domain of KIR2DL4 is selected from a group consisting of: A238P and S267fs. In some embodiments, the mutation results in a change in amino acid in KIR2DL4 (SEQ ID NO.: 5) selected from a group consisting of: R50, H52, R55, N58, T61, K65, Q149, I154, E162, L166, I174, A238, and S267. In some embodiments, the mutation is or comprises a modification in a codon of KIR2DL4 encoding an amino acid R50. In some embodiments, the mutation is or comprises a modification in a codon of KIR2DL4 encoding the amino acid H52. In some embodiments, the mutation is or comprises a modification in a codon of KIR2DL4 encoding the amino acid R55. In some embodiments, the mutation is or comprises a modification in a codon of KIR2DL4 encoding the amino acid N58. In some embodiments, the mutation is or comprises a modification in a codon of KIR2DL4 encoding the amino acid T61. In some embodiments, the mutation is or comprises a modification in a codon of KIR2DL4 encoding the amino acid K65. In some embodiments, the mutation is or comprises a modification in a codon of KIR2DL4 encoding the amino acid Q149. In some embodiments, the mutation is or comprises a modification in a codon of KIR2DL4 encoding the amino acid I154. In some embodiments, the mutation is or comprises a modification in a codon of KIR2DL4 encoding the amino acid E162. In some embodiments, the mutation is or comprises a modification in a codon of KIR2DL4 encoding the amino acid L166. In some embodiments, the mutation is or comprises a modification in a codon of KIR2DL4 encoding the amino acid I174. In some embodiments, the mutation is or comprises a modification in a codon of KIR2DL4 encoding the amino acid A238. In some embodiments, the mutation is or comprises a modification in a codon of KIR2DL4 encoding the amino acid S267. In some embodiments, the mutation results in a change (e.g., a substitution or deletion) in amino acid Q149 (e.g., Q149K mutation) of KIR2DL4. In some embodiments, the mutation in KIR2DL4 is R50L. In some embodiments, the mutation in KIR2DL4 is H52R. In some embodiments, the mutation in KIR2DL4 is R55L. In some embodiments, the mutation in KIR2DL4 is N58T. In some embodiments, the mutation in KIR2DL4 is T61R. In some embodiments, the mutation in KIR2DL4 is K65E. In some embodiments, the mutation in KIR2DL4 is Q149K. In some embodiments, the mutation in KIR2DL4 is Q149R. In some embodiments, the mutation in KIR2DL4 is I154M. In some embodiments, the mutation in KIR2DL4 is E162K. In some embodiments, the mutation in KIR2DL4 is E162G. In some embodiments, the mutation in KIR2DL4 is L166P. In some embodiments, the mutation in KIR2DL4 is I174V. In some embodiments, the mutation in KIR2DL4 is A238P. In some embodiments, the mutation in KIR2DL4 is S267fs. In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, the method of treating a cancer in a subject in need thereof, comprises administering a therapeutically effective amount of an FTI, optionally tipifarnib, to said subject, wherein the cancer is a cancer known to have or determined to have a mutation in KIR3DL1, such as two, three, four, or more mutations, in KIR3DL1. In some embodiments, the methods provided herein include determining the presence of the mutation in KIR3DL1 (e.g., determining the presence of the two, three, four, or more mutations, in KIR3DL1) in a sample from a subject having cancer, and administering a therapeutically effective amount of an FTI to said subject if the mutation in KIR3DL1 is present (e.g., if the two, three, four, or more mutations, in KIR3DL1 are present). In some embodiments, the mutation is or comprises a modification in a codon of KIR3DL1 encoding an amino acid in the extracellular domain, in the cytoplasmic domain, or combinations thereof. In some embodiments, the methods provided herein include determining the presence of has two, three, four, or more, mutations in the KIR3DL1 comprising two, three, four, or more, modifications at two, three, four, or more, codons that endode two, three, four, or more, amino acids in the extracellular domain, at two, three, four, or more, codons that endode two, three, four, or more, amino acids in the cytoplasmic domain, or combinations thereof. In some embodiments, the mutation is or comprises a modification in a codon of KIR3DL1 encoding an amino acid selected from a group consisting of: R292, F297, P336, R409, R413, I426, L427, T429, and V440. In some embodiments, the mutation in KIR3DL1 is selected from a group consisting of: R292T, F297L, P336R, R409T, R413C, I426T, L427M, T429M, and V440I. In some embodiments, the mutation is or comprises a modification in a codon of KIR3DL1 encoding an amino acid selected from a group consisting of: R292, F297, I426, L427, and T429. In some embodiments, the mutation in KIR3DL1 is selected from a group consisting of: R292T, F297L, I426T, L427M, and T429M. In some embodiments, the mutation is or comprises a modification in a codon of KIR3DL1 encoding an amino acid in the extracellular domain selected from a group consisting of: R292, F297, and P336. In some embodiments, the mutation in the extracellular domain of KIR3DL1 is selected from a group consisting of: R292T, F297L, and P336R. In some embodiments, the mutation is or comprises a modification in a codon of KIR3DL1 encoding the amino acid R292 and/or F297 in the extracellular domain. In some embodiments, the mutation in the extracellular domain of KIR3DL1 is or comprises the R292T and/or the F297L. In some embodiments, the mutation is or comprises a modification in a codon of KIR3DL1 encoding an amino acid in the cytoplasmic domain selected from a group consisting of: R409, R413, I426, L427, T429, and V440. In some embodiments, the mutation in the cytoplasmic domain of KIR3DL1 is selected from a group consisting of: R409T, R413C, I426T, L427M, T429M, and V440I. In some embodiments, the mutation in the cytoplasmic domain of KIR3DL1 is within or near the PKC site, the PDK site, and/or the immunoreceptor tyrosine-based inhibitory motif 2 (ITIM 2), of said cytoplasmic domain. In some embodiments, the mutation is or comprises a modification in a codon of KIR3DL1 encoding the amino acid within or near PKC site of the cytoplasmic domain, such as R409 and/or R413. In some embodiments, the mutation within or near the PKC site of the cytoplasmic domain of KIR3DL1 is or comprises R409T and/or R413C. In some embodiments, the mutation is or comprises a modification in a codon of KIR3DL1 encoding the amino acid within or near the ITIM 2 of the cytoplasmic domain selected from a group consisting of: I426, L427, and T429. In some embodiments, the mutation within or near the ITIM 2 of the cytoplasmic domain of KIR3DL1 is selected from a group consisting of: I426T, L427M, and T429M. In some embodiments, the mutation results in a change in amino acid in KIR3DL1 (SEQ ID NO.: 7) selected from a group consisting of: R292, F297, P336, R409, R413, I426, L427, T429, and V440. In some embodiments, the mutation is or comprises a modification in a codon of KIR3DL1 encoding the amino acid R292. In some embodiments, the mutation is or comprises a modification in a codon of KIR3DL1 encoding the amino acid F297. In some embodiments, the mutation is or comprises a modification in a codon of KIR3DL1 encoding the amino acid P336. In some embodiments, the mutation is or comprises a modification in a codon of KIR3DL1 encoding the amino acid R409. In some embodiments, the mutation is or comprises a modification in a codon of KIR3DL1 encoding the amino acid R413. In some embodiments, the mutation is or comprises a modification in a codon of KIR3DL1 encoding the amino acid I426. In some embodiments, the mutation is or comprises a modification in a codon of KIR3DL1 encoding the amino acid L427. In some embodiments, the mutation is or comprises a modification in a codon of KIR3DL1 encoding the amino acid T429. In some embodiments, the mutation is or comprises a modification in a codon of KIR3DL1 encoding an amino acid V440. In some embodiments, the mutation results in a change (e.g., a substitution or deletion) in amino acid R292 (e.g., R292T mutation) of KIR3DL1. In some embodiments, the mutation in KIR3DL1 is R292T. In some embodiments, the mutation in KIR3DL1 is F297L. In some embodiments, the mutation in KIR3DL1 is P336R. In some embodiments, the mutation in KIR3DL1 is R409T. In some embodiments, the mutation in KIR3DL1 is R413C. In some embodiments, the mutation in KIR3DL1 is I426T. In some embodiments, the mutation in KIR3DL1 is L427M. In some embodiments, the mutation in KIR3DL1 is T429M. In some embodiments, the mutation in KIR3DL1 is V440I. In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, the method of treating a cancer in a subject in need thereof, comprises administering a therapeutically effective amount of an FTI, optionally tipifarnib, to said subject, wherein the cancer is a cancer known to have or determined to have a mutation in KIR3DL2, such as two, three, four, or more mutations, in KIR3DL2. In some embodiments, the methods provided herein include determining the presence of the mutation in KIR3DL2 (e.g., determining the presence of the two, three, four, or more mutations, in KIR3DL2) in a sample from a subject having cancer, and administering a therapeutically effective amount of an FTI to said subject if the mutation in KIR3DL2 is present (e.g., if the two, three, four, or more mutations, in KIR3DL2 are present). In some embodiments, the mutation is or comprises a modification in a codon of KIR3DL2 encoding an amino acid in the extracellular domain, in the cytoplasmic domain, or combinations thereof. In some embodiments, the methods provided herein include determining the presence of has two, three, four, or more, mutations in the KIR3DL2 comprising two, three, four, or more, modifications at two, three, four, or more, codons that endode two, three, four, or more, amino acids in the extracellular domain, at two, three, four, or more, codons that endode two, three, four, or more, amino acids in the cytoplasmic domain, or combinations thereof. In some embodiments, the mutation is or comprises a modification in a codon of KIR3DL2 encoding an amino acid selected from a group consisting of: P319, W323, P324, S333, C336, V341, and Q386. In some embodiments, the mutation in KIR3DL2 is selected from a group consisting of: P319S, W323S, P324S, S333T, C336R, V341I, and Q386E. In some embodiments, the mutation is or comprises a modification in a codon of KIR3DL2 encoding the amino acid C336 and/or Q386. In some embodiments, the mutation in KIR3DL2 is or comprises the C336R and/or the Q386E. In some embodiments, the mutation is or comprises a modification in a codon of KIR3DL2 encoding an amino acid in the extracellular domain selected from a group consisting of: P319, W323, P324, S333, C336, and V341. In some embodiments, the mutation in the extracellular domain of KIR3DL2 is selected from a group consisting of: P319S, W323S, P324S, S333T, C336R, and V341I. In some embodiments, the mutation is or comprises a modification in a codon of KIR3DL2 encoding the extracellular domain amino acid C336. In some embodiments, the mutation in the extracellular domain of KIR3DL2 is C336R. In some embodiments, the mutation is or comprises a modification in a codon of KIR3DL2 encoding the cytoplasmic domain amino acid Q386. In some embodiments, the mutation in the cytoplasmic domain of KIR3DL2 is Q386E. In some embodiments, the mutation results in a change in amino acid in KIR3DL2 (SEQ ID NO.: 9) selected from a group consisting of: P319, W323, P324, S333, C336, V341, and Q386. In some embodiments, the mutation is or comprises a modification in a codon of KIR3DL2 encoding the amino acid P319. In some embodiments, the mutation is or comprises a modification in a codon of KIR3DL2 encoding the amino acid W323. In some embodiments, the mutation is or comprises a modification in a codon of KIR3DL2 encoding the amino acid P324. In some embodiments, the mutation is or comprises a modification in a codon of KIR3DL2 encoding the amino acid S333. In some embodiments, the mutation is or comprises a modification in a codon of KIR3DL2 encoding the amino acid C336. In some embodiments, the mutation is or comprises a modification in a codon of KIR3DL2 encoding the amino acid V341. In some embodiments, the mutation is or comprises a modification in a codon of KIR3DL2 encoding the amino acid Q386. In some embodiments, the mutation results in a change (e.g., a substitution or deletion) in amino acid Q386 (e.g., Q386E mutation) of KIR3DL2. In some embodiments, the mutation in KIR3DL2 is P319S. In some embodiments, the mutation in KIR3DL2 is W323S. In some embodiments, the mutation in KIR3DL2 is P324S. In some embodiments, the mutation in KIR3DL2 is S333T. In some embodiments, the mutation in KIR3DL2 is C336R. In some embodiments, the mutation in KIR3DL2 is V341I. In some embodiments, the mutation in KIR3DL2 is Q386E. In some embodiments, the method further provides determining the VAF of a KIR3DL2 mutation from the sample from the cancer subject. In some embodiments, the method further provides determining the VAF of the KIR3DL2 mutation selected from the group consisting of: a KIR3DL2 C336R mutation, a KIR3DL2 Q386E mutation, or a KIR3DL2 C336R/Q386E mutation, from the sample from the cancer subject, such as wherein the cancer is AITL, for example, wherein the cancer is relapsed or refractory AITL. In some embodiments, the VAF is determined by a NGS assay. In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, the method of treating a cancer in a subject in need thereof, comprises administering a therapeutically effective amount of an FTI, optionally tipifarnib, to said subject, wherein the cancer is a cancer known to have or determined to have a mutation in KIR2DL3 and KIR3DL2, such as two, three, four, or more mutations, in KIR2DL3 and KIR3DL2. In some embodiments, the methods provided herein include determining the presence of the mutation(s) in KIR2DL3 and KIR3DL2 (e.g., determining the presence of the two, three, four, or more mutations, in KIR2DL3 and KIR3DL2) in a sample from a subject having cancer, and administering a therapeutically effective amount of an FTI to said subject if the mutations in KIR2DL3 and KIR3DL2 are present (e.g., if the two, three, four, or more mutations, in KIR2DL3 and KIR3DL2 are present). In some embodiments, the mutation(s) in KIR2DL3 and KIR3DL2 is or comprises a modification in a codon that encodes an amino acid in the extracellular domain, in the cytoplasmic domain, or combinations thereof, of the KIR2DL3 and KIR3DL2. In some embodiments, the mutation is or comprises a modification in a codon of KIR2DL3 encoding the amino acid R162 and/or E295, and the mutation is or comprises a modification in a codon of KIR3DL2 encoding the amino acid C336 and/or Q386. In some embodiments, the mutation in KIR2DL3 is or comprises R162T and/or E295D, and the mutation in KIR3DL2 is or comprises C336R and/or Q386E. In specific embodiments, the cancer is hematological (or hematogenous) cancer (e.g., leukemia, lymphoma, myeloproliferative neoplasm (MPN), or myelodysplastic syndrome (MDS)) or a solid tumor. In specific embodiments, the cancer is a solid tumor. In specific embodiments, the cancer is lymphoma. In specific embodiments, the cancer is T-cell lymphoma. In specific embodiments, the cancer is PTCL. In specific embodiments, the cancer is AITL. In specific embodiments, the cancer is CTCL. In specific embodiments, the cancer is relapsed or refractory PTCL. In specific embodiments, the cancer is PTCL-NOS. In specific embodiments, the cancer is relapsed or refractory AITL. In specific embodiments, the cancer is AITL-NOS. In specific embodiments, the cancer is ALCL-ALK positive. In specific embodiments, the cancer is ALCL-ALK negative. In specific embodiments, the cancer is enteropathy-associated T-cell lymphoma. In specific embodiments, the cancer is NK lymphoma. In specific embodiments, the cancer is extranodal natural killer cell (NK) T-cell lymphoma—nasal type. In specific embodiments, the cancer is hepatosplenic T-cell lymphoma. In specific embodiments, the cancer is subcutaneous panniculitis-like T-cell lymphoma. In specific embodiments, the cancer is EBV associated lymphoma. In specific embodiments, the cancer is leukemia. In specific embodiments, the cancer is NK leukemia. In specific embodiments, the cancer is AML. In specific embodiments, the leukemia is T-ALL. In specific embodiments, the cancer is CML. In specific embodiments, the cancer is MDS. In specific embodiments, the cancer is MPN. In specific embodiments, the cancer is CMML. In specific embodiments, the cancer is JMML.

In some embodiments, the KIR-mutant cancer can include at least one mutation that is or comprises a modification in a codon that encodes an amino acid selected from the group consisting of M65, H77, A83, S88, T91, L140, N178, G179, D184, R197, F202, and H203 of KIR2DL1 (SEQ ID NO:1). In some embodiments, the KIR-mutant cancer can include at least two mutations that are or comprise modifications in codons that encode amino acids selected from the group consisting of M65, H77, A83, S88, T91, L140, N178, G179, D184, R197, F202, and H203 of KIR2DL1 (SEQ ID NO:1).

The mutations in a KIR2DL1 gene can be point mutations resulting in an amino acid substitution or can be frameshift mutations (fs) resulting in a shift of the reading frame. For example, a mutation in a KIR2DL1 gene can be a mutation leading to substitution of an amino acid M65, H77, A83, S88, T91, L140, N178, G179, D184, R197, F202, or H203 of KIR2DL1 (SEQ ID NO:1).

In some embodiments, the KIR-mutant cancer can include at least one mutation that is or comprises a modification in a codon that encodes an amino acid selected from the group consisting of: F66, R162, R169, F171, S172, E295, R318, I330, I331, and V332 of KIR2DL3 (SEQ ID NO:1). In some embodiments, the KIR-mutant cancer can include at least two mutations that are or comprise modifications in codons that encode amino acids selected from the group consisting of F66, R162, R169, F171, S172, E295, R318, I330, I331, and V332 of KIR2DL3 (SEQ ID NO:3).

The mutations in a KIR2DL3 gene can be point mutations resulting in an amino acid substitution or can be frameshift mutations (fs) resulting in a shift of the reading frame. For example, a mutation in a KIR2DL3 gene can be a mutation leading to substitution of an amino acid F66, R162, R169, F171, S172, E295, R318, I330, I331, or V332 of KIR2DL3 (SEQ ID NO: 3).

In some embodiments, the KIR-mutant cancer can include at least one mutation that is or comprises a modification in a codon that encodes an amino acid selected from the group consisting of: R50, H52, R55, N58, T61, K65, Q149, I154, E162, L166, I174, A238, and S267 of KIR2DL4 (SEQ ID NO:1). In some embodiments, the KIR-mutant cancer can include at least two mutations that are or comprise modifications in codons that encode amino acids selected from the group consisting of R50, H52, R55, N58, T61, K65, Q149, I154, E162, L166, I174, A238, and S267 of KIR2DL4 (SEQ ID NO:5).

The mutations in a KIR2DL4 gene can be point mutations resulting in an amino acid substitution or can be frameshift mutations (fs) resulting in a shift of the reading frame. For example, a mutation in a KIR2DL4 gene can be a mutation leading to substitution of an amino acid R50, H52, R55, N58, T61, K65, Q149, I154, E162, L166, I174, A238, or S267 of KIR2DL4 (SEQ ID NO: 5).

In some embodiments, the KIR-mutant cancer can include at least one mutation that is or comprises a modification in a codon that encodes an amino acid selected from the group consisting of: R292, F297, P336, R409, R413, I426, L427, T429, and V440 of KIR3DL1 (SEQ ID NO:1). In some embodiments, the KIR-mutant cancer can include at least two mutations that are or comprise modifications in codons that encode amino acids selected from the group consisting of R292, F297, P336, R409, R413, I426, L427, T429, and V440 of KIR3DL1 (SEQ ID NO: 7).

The mutations in a KIR3DL1 gene can be point mutations resulting in an amino acid substitution or can be frameshift mutations (fs) resulting in a shift of the reading frame. For example, a mutation in a KIR3DL1 gene can be a mutation leading to substitution of an amino acid R292, F297, P336, R409, R413, I426, L427, T429, or V440 of KIR3DL1 (SEQ ID NO: 7).

In some embodiments, the KIR-mutant cancer can include at least one mutation that is or comprises a modification in a codon that encodes an amino acid selected from the group consisting of: P319, W323, P324, S333, C336, V341, and Q386 of KIR3DL2 (SEQ ID NO:1). In some embodiments, the KIR-mutant cancer can include at least two mutations that are or comprise modifications in codons that encode amino acids selected from the group consisting of P319, W323, P324, S333, C336, V341, and Q386 of KIR3DL2 (SEQ ID NO: 9).

The mutations in a KIR3DL2 gene can be point mutations resulting in an amino acid substitution or can be frameshift mutations (fs) resulting in a shift of the reading frame. For example, a mutation in a KIR3DL2 gene can be a mutation leading to substitution of an amino acid P319, W323, P324, S333, C336, V341, or Q386 of KIR3DL2 (SEQ ID NO: 9).

In some embodiments, the cancer treated in accordance with the methods described herein has a mutation in a gene encoding SEQ ID NO:1 or carries a mutant SEQ ID NO:1. In some embodiments, the cancer treated in accordance with the methods described herein has a mutation in a gene encoding SEQ ID NO:3 or carries a mutant SEQ ID NO:3. In some embodiments, the cancer treated in accordance with the methods described herein has a mutation in a gene encoding SEQ ID NO:5 or carries a mutant SEQ ID NO:5. In some embodiments, the cancer treated in accordance with the methods described herein has a mutation in a gene encoding SEQ ID NO:7 or carries a mutant SEQ ID NO:7. In some embodiments, the cancer treated in accordance with the methods described herein has a mutation in a gene encoding SEQ ID NO:9 or carries a mutant SEQ ID NO:9.

In some embodiments, a sample from the subject treated in accordance with the methods described herein is detected to have a mutation in a gene encoding SEQ ID NO:1 or a mutant SEQ ID NO:1. In some embodiments, a sample from the subject treated in accordance with the methods described herein is detected to have a mutation in a gene encoding SEQ ID NO:3 or a mutant SEQ ID NO:3. In some embodiments, a sample from the subject treated in accordance with the methods described herein is detected to have a mutation in a gene encoding SEQ ID NO:5 or a mutant SEQ ID NO:5. In some embodiments, a sample from the subject treated in accordance with the methods described herein is detected to have a mutation in a gene encoding SEQ ID NO:7 or a mutant SEQ ID NO:7. In some embodiments, a sample from the subject treated in accordance with the methods described herein is detected to have a mutation in a gene encoding SEQ ID NO:9 or a mutant SEQ ID NO:9.

In some embodiments, the subject treated in accordance with the methods described herein has two or more mutations in one or more genes of the KIR family (e.g., two, three, four, five or six mutations in one or more of KIR2DL1, KIR2DL3, KIR2DL4, KIR3DL1, and/or KIR3DL2). In some embodiments, the subject treated in accordance with the methods described herein has one or more mutations in two or more genes of the KIR family (e.g., one or more mutations in two or more KIR2DL1, KIR2DL3, KIR2DL4, KIR3DL1, and KIR3DL2 genes).

Provided herein are methods for predicting responsiveness of a cancer patient to an FTI treatment, methods for cancer patient population selection for an FTI treatment, and methods for treating cancer in a subject with a therapeutically effective amount of an FTI, based on the mutation status of KIR2DL1, KIR2DL3, KIR2DL4, KIR3DL1, and/or KIR3DL2 in a sample from the patient. In some embodiments, the method includes determining the presence or absence of a mutation in KIR2DL1, KIR2DL3, KIR2DL4, KIR3DL1, and/or KIR3DL2 in a sample from the subject prior to beginning treatment. In some embodiments, patients are selected based on the presence of a KIR2DL1, KIR2DL3, KIR2DL4, KIR3DL1, and/or KIR3DL2 mutation. Tumors or cancers that have a KIR2DL1, KIR2DL3, KIR2DL4, KIR3DL1, and/or KIR3DL2 mutation indicate that the patients will likely be responsive to the FTI treatment.

Provided herein are methods for predicting responsiveness of a cancer patient to an FTI treatment, methods for cancer patient population selection for an FTI treatment, and methods for treating cancer in a subject with a therapeutically effective amount of an FTI, based on the mutation status of KIR (e.g., KIR2DL1, KIR2DL3, KIR2DL4, KIR3DL1, and/or KIR3DL2) in a sample from the patient. In some embodiments, the method includes determining the presence or absence of a mutation in KIR (e.g., KIR2DL1, KIR2DL3, KIR2DL4, KIR3DL1, and/or KIR3DL2) in a sample from the subject prior to beginning treatment. In some embodiments, patients are selected based on the presence of a KIR mutation (e.g., mutation of KIR2DL1, KIR2DL3, KIR2DL4, KIR3DL1, and/or KIR3DL2). Tumors or cancers that have a KIR mutation (e.g., mutations of KIR2DL1, KIR2DL3, KIR2DL4, KIR3DL1, and/or KIR3DL2) indicate that the patients will likely be responsive to the FTI treatment.

As a person of ordinary skill in the art would understand, any methods described herein or otherwise known in the art for analyzing mutations can be used for determining the presence or absence of a mutation in KIR2DL1, KIR2DL3, KIR2DL4, KIR3DL1, and/or KIR3DL2. The mutation status of KIR2DL1, KIR2DL3, KIR2DL4, KIR3DL1, and/or KIR3DL2 can be detected at the nucleic acid or protein level. In some embodiments, the mutation status is determined by analyzing nucleic acids obtained from the sample. In some embodiments, the mutation status is determined by analyzing protein obtained from the sample.

In some embodiments, the mutation status of KIR2DL1, KIR2DL3, KIR2DL4, KIR3DL1, and/or KIR3DL2 is determined by analyzing nucleic acids obtained from the sample. In some embodiments, the determined mutation status of KIR2DL1, KIR2DL3, KIR2DL4, KIR3DL1, and/or KIR3DL2 is a variant allele frequency (VAF). The nucleic acids may be mRNA or genomic DNA molecules from the test subject. Methods for determining the mutation status by analyzing nucleic acids are well known in the art. In some embodiments, the methods include sequencing, Polymerase Chain Reaction (PCR), DNA microarray, Mass Spectrometry (MS), Single Nucleotide Polymorphism (SNP) assay, denaturing high-performance liquid chromatography (DHPLC), or Restriction Fragment Length Polymorphism (RFLP) assay. In some embodiments, the mutation status is determined using standard sequencing methods, including, for example, Sanger sequencing, next generation sequencing (NGS). In some embodiments, the mutation status is determined using MS.

In some embodiments, the method includes determining the presence or absence of a KIR2DL1, KIR2DL3, KIR2DL4, KIR3DL1, and/or KIR3DL2 mutation by amplifying the respective KIR2DL1, KIR2DL3, KIR2DL4, KIR3DL1, and/or KIR3DL2 nucleic acid from a sample by PCR. For example, PCR technology and primer pairs that can be used are known to the person skilled in the art. In some embodiments, primers selected for gene amplification evaluation are highly specific to avoid detecting closely related homologous genes. Following multiplex PCR amplification, the products can be purified to remove the primers and unincorporated deoxynucleotide triphosphates using PCR-M™ Clean Up System (Viogenebiotek Co., Sunnyvale, Calif., USA). Purified DNA can then be semiquantified on a 1% agarose gel in 0.5×TBE and visualized by staining with ethidium bromide. The products can then be subjected to primer extension analysis. The primer extension reaction products can then be resolved by automated capillary electrophoresis on a capillary electrophoresis platform, e.g. 14 μl of Hi-Di™ Formamide (Applied Biosystems) and 0.28 μl of GeneScan™-120LIZ® Size Standard (Applied Biosystems) were added to 6 μl of primer extension products. All samples may then e.g. be analyzed on an ABI Prism 310 DNA Genetic Analyzer (Applied Biosystems) according to manufacturer's instructions using GeneScan™ 3.1 (Applied Biosystems).

Provided herein are methods of selecting a cancer patient who is likely to benefit from an FTI treatment, include determining the presence or absence of a KIR2DL1, KIR2DL3, KIR2DL4, KIR3DL1, and/or KIR3DL2 mutation by amplifying the respective KIR2DL1, KIR2DL3, KIR2DL4, KIR3DL1, and/or KIR3DL2 nucleic acid from the patient's tumor sample and sequencing the amplified nucleic acid.

In the methods provided herein, KIR2DL1, KIR2DL3, KIR2DL4, KIR3DL1, and/or KIR3DL2 nucleic acid can be obtained from the patient's tumor sample by any method known to the person skilled in the art. For example, any commercial kit may be used to isolate the genomic DNA, or mRNA from a tumor sample, such as e.g. the Qlamp DNA mini kit, or RNeasy mini kit (Qiagen, Hilden, Germany). For example, if mRNA was isolated from the patient's tumor sample, cDNA synthesis can be carried out prior to the methods as disclosed herein, according to any known technology in the art.

For example, the nucleic acid to be isolated from a tumor can for example be one of genomic DNA, total RNA, mRNA or poly(A)+ mRNA. For example, if mRNA has been isolated from the patient's tumor sample, the mRNA (total mRNA or poly(A)+ mRNA) may be used for cDNA synthesis according to well established technologies in prior art, such as those provided in commercial cDNA synthesis kits, e.g. Superscript® III First Strand Synthesis Kit. The cDNA can then be further amplified by means of e.g. PCR and subsequently subjected to sequencing by e.g. Sanger sequencing or pyro-sequencing to determine the nucleotide sequence of KIR2DL1, KIR2DL3, KIR2DL4, KIR3DL1, and/or KIR3DL2 gene. Alternatively, the PCR product can e.g. also be subcloned into a TA TOPO cloning vector for sequencing. Other technologies than sequencing to determine the absence or presence of KIR2DL1, KIR2DL3, KIR2DL4, KIR3DL1, and/or KIR3DL2 mutations can be used in the methods provided herein such as e.g. Single Nucleotide Primer Extension (SNPE) (PLoS One. 2013 Aug. 21; 8(8):e72239); DNA microarray, Mass Spectrometry (MS) (e.g. matrix-assisted laser desorption/ionization time-of-flight (MALDI-TOF) mass spectrometry), Single Nucleotide Polymorphism (SNP), denaturing high-performance liquid chromatography (DHPLC), or Restriction Fragment Length Polymorphism (RFLP) assay.

For example, Single Nucleotide Polymorphism (SNP) Assay can be used for determining KIR2DL1, KIR2DL3, KIR2DL4, KIR3DL1, and/or KIR3DL2 mutation status in a sample. The SNP assay can be performed on the HT7900 from Applied Biosystems, following the allelic discrimination assay protocol provided by the manufacturer. KIR2DL1, KIR2DL3, KIR2DL4, KIR3DL1, and/or KIR3DL2 mutation status can also be determined by DHPLC or RFLP, or any other methods known in the art. Bowen et al., Blood, 106:2113-2119 (2005); Bowen et al., Blood, 101:2770-2774 (2003); Nishikawa et al., Clin Chim Acta., 318:107-112 (2002); Lin S Y et al., Am J Clin Pathol. 100:686-689 (1993); O'Leary J J et al., J Clin Pathol. 51:576-582 (1998).

In some embodiments, the mutation status of KIR2DL1, KIR2DL3, KIR2DL4, KIR3DL1, and/or KIR3DL2 is determined by analyzing protein obtained from the sample. The mutated protein can be detected by a variety of immunohistochemistry (IHC) approaches, Immunoblotting assay, Enzyme-Linked Immunosorbent Assay (ELISA) or other immunoassay methods known in the art.

IHC staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Thus, antibodies or antisera, preferably polyclonal antisera, and most preferably monoclonal antibodies that specifically target mutant KIR2DL1, KIR2DL3, KIR2DL4, KIR3DL1, and/or KIR3DL2 can be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available. Automated systems for slide preparation and IHC processing are available commercially. The Ventana® BenchMark XT system is an example of such an automated system.

Standard immunological and immunoassay procedures can be found in Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Ten, eds., 7th ed. 1991).

Assays to detect KIR2DL1, KIR2DL3, KIR2DL4, KIR3DL1, and/or KIR3DL2 mutations include noncompetitive assays, e.g., sandwich assays, and competitive assays. Typically, an assay such as an ELISA assay can be used. ELISA assays are known in the art, e.g., for assaying a wide variety of tissues and samples, including blood, plasma, serum or bone marrow.

A wide range of immunoassay techniques using such an assay format are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target mutant KIR2DL1, KIR2DL3, KIR2DL4, KIR3DL1, and/or KIR3DL2 protein. Sandwich assays are commonly used assays. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the mutant KIR protein is either covalently or passively bound to a solid surface. The solid surface may be glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the mutant KIR protein. The second antibody is linked to a reporter molecule which is used to indicate the binding of the second antibody to the mutant KIR protein.

In some embodiments, flow cytometry (FACS) can be used to detect the mutant KIR2DL1, KIR2DL3, KIR2DL4, KIR3DL1, and/or KIR3DL2 using antibodies that specifically target the mutant KIR2DL1, KIR2DL3, KIR2DL4, KIR3DL1, and/or KIR3DL2. The flow cytometer detects and reports the intensity of the fluorichrome-tagged antibody, which indicates the presence of the mutant KIR2DL1, KIR2DL3, KIR2DL4, KIR3DL1, and/or KIR3DL2. Non-fluorescent cytoplasmic proteins can also be observed by staining permeablized cells. The stain can either be a fluorescence compound able to bind to certain molecules, or a fluorichrome-tagged antibody to bind the molecule of choice.

An alternative method involves immobilizing the target KIR2DL1, KIR2DL3, KIR2DL4, KIR3DL1, and/or KIR3DL2 protein in the sample and then exposing the immobilized target to mutant specific antibody which may or may not be labeled with a reporter molecule. Depending on the amount of target and the strength of the reporter molecule signal, a bound target can be detectable by direct labeling with the antibody. Alternatively, a second labeled antibody, specific to the first antibody is exposed to the target-first antibody complex to form a target-first antibody-second antibody tertiary complex. The complex is detected by the signal emitted by a labeled reporter molecule.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, and other are discussed herein. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of mutant KIR2DL1, KIR2DL3, KIR2DL4, KIR3DL1, and/or KIR3DL2 protein which was present in the sample. Alternately, fluorescent compounds, such as fluorescein and rhodamine, can be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art and are discussed herein.

In some embodiments, the determination of the KIR2DL1, KIR2DL3, KIR2DL4, KIR3DL1, and/or KIR3DL2 mutation status is performed as a companion diagnostic to the FTI treatment. The companion diagnostic can be performed at the clinic site where the subject is treated. The companion diagnostic can also be performed at a site separate from the clinic site where the subject is treated.

As a person of ordinary skill in the art would understand, methods provided herein are for predicting responsiveness of a cancer patient to an FTI treatment, methods for cancer patient population selection for an FTI treatment, and methods for treating cancer in a subject with a therapeutically effective amount of an FTI, based on the mutation status of KIR2DL1, KIR2DL3, KIR2DL4, KIR3DL1, and/or KIR3DL2 in a sample from the patient. Any methods described herein or otherwise known in the art for determining the mutation status of KIR2DL1, KIR2DL3, KIR2DL4, KIR3DL1, and/or KIR3DL2 can be applied. In a preferred embodiment, methods provided herein are for predicting responsiveness of a cancer patient to an FTI treatment, methods for cancer patient population selection for an FTI treatment, and methods for treating cancer in a subject with a therapeutically effective amount of an FTI, based on the mutation status of KIR in a sample from the patient.

As provided herein, the genotype of a KIR mutant (e.g., KIR2DL1, KIR2DL3, KIR2DL4, KIR3DL1, and/or KIR3DL2) of a subject can be indicative of the likelihood of the subject to respond to an FTI treatment. A cancer patient who is a carrier of a mutation in KIR2DL1, KIR2DL3, KIR2DL4, KIR3DL1, and/or KIR3DL2 is likely to be responsive to an FTI treatment. Accordingly, KIR2DL1, KIR2DL3, KIR2DL4, KIR3DL1, and/or KIR3DL2 typing cancer patients, and selectively treating those who are carriers of one or more mutations in KIR2DL1, KIR2DL3, KIR2DL4, KIR3DL1, and/or KIR3DL2, can increase the overall response rate of the cancer patients to an FTI treatment.

As provided herein, the VAF of a KIR mutant (e.g., KIR2DL1, KIR2DL3, KIR2DL4, KIR3DL1, and/or KIR3DL2) of a cancer subject (such as from a sample of the cancer subject) can be indicative of the likelihood of the cancer subject to respond to an FTI treatment. In some embodiments, a cancer subject having a KIR3DL2 C336R mutation VAF of greater than 10%, greater than 15%, or greater than 20%, is likely to be responsive to an FTI treatment. In some embodiments, a cancer subject having a KIR3DL2 Q386E mutation VAF of greater than 5%, greater than 6%, greater than 7%, greater than 8%, or greater than 9%, is likely to be responsive to an FTI treatment. In some embodiments, a cancer subject having a KIR3DL2 C336R/Q386E mutation, with a KIR3DL2 C336R mutation VAF of greater than 10%, greater than 15%, or greater than 20%, and a KIR3DL2 Q386E mutation VAF of greater than 5%, greater than 6%, greater than 7%, greater than 8%, or greater than 9%, is likely to be responsive to an FTI treatment. In specific embodiments, the KIR3DL2 C336R mutation VAF of a subject is greater than 10%. In specific embodiments, the KIR3DL2 C336R mutation VAF of a subject is greater than 15%. In specific embodiments, the KIR3DL2 C336R mutation VAF of a subject is greater than 20%. In specific embodiments, the KIR3DL2 Q386E mutation VAF of a subject is greater than 6%. In specific embodiments, the KIR3DL2 Q386E mutation VAF of a subject is greater than 7%. In specific embodiments, the KIR3DL2 Q386E mutation VAF of a subject is greater than 8%. In specific embodiments, the KIR3DL2 Q386E mutation VAF of a subject is greater than 9%. In specific embodiments, the VAF is determined by NGS. Accordingly, KIR3DL2 typing cancer subjects, and selectively treating those who are carriers of mutations in KIR3DL2, with a KIR3DL2 C336R mutation VAF of greater than 10%, greater than 15%, or greater than 20%, and/or with a KIR3DL2 Q386E mutation VAF of greater than 5%, greater than 6%, greater than 7%, greater than 8%, or greater than 9%, can increase the overall response rate of the cancer patients to an FTI treatment. In some embodiments, the AITL is refractory and resistant to a prior standard of care (SOC) treatment selected from the group consisting of: Nivolumab, BEAM/ASCT, DICE, CHOP-E, Brentuximab ved., CEOP, and GemDOX. In some embodiments, the refractory and resistant AITL has a KIR3DL2 Q386E mutation VAF of greater than 5%, 6%, 7%, 8%, or 9%. In some embodiments, the refractory and resistant AITL has a KIR3DL2 Q386E mutation VAF of greater than 5%. In some embodiments, the subject has an improved overall response rate to tipifarnib administration relative to the overall response rate of the prior SOC treatment.

In some embodiments, the subject who is a carrier of a KIR2DL1 is homozygous for that mutation. In some embodiments, the subject who is a carrier of a KIR2DL1 mutation is heterozygous for that mutation. In some embodiments, the subject who is a carrier of a KIR2DL3 is homozygous for that mutation. In some embodiments, the subject who is a carrier of a KIR2DL3 mutation is heterozygous for that mutation. In some embodiments, the subject who is a carrier of a KIR2DL4 is homozygous for that mutation. In some embodiments, the subject who is a carrier of a KIR2DL4 mutation is heterozygous for that mutation. In some embodiments, the subject who is a carrier of a KIR3DL1 is homozygous for that mutation. In some embodiments, the subject who is a carrier of a KIR3DL1 mutation is heterozygous for that mutation. In some embodiments, the subject who is a carrier of a KIR3DL2 is homozygous for that mutation. In some embodiments, the subject who is a carrier of a KIR3DL2 mutation is heterozygous for that mutation.

The methods provided herein can be performed by any method described herein or otherwise known in the art. In some embodiments, provided herein is a method for treating cancer in a subject with an FTI by KIR typing, or selecting a cancer patient for an FTI treatment by KIR typing, wherein the KIR typing is performed by sequencing, Polymerase Chain Reaction (PCR), DNA microarray, Mass Spectrometry (MS), Single Nucleotide Polymorphism (SNP) assay, Immunoblotting assay, or Enzyme-Linked Immunosorbent Assay (ELISA). In some embodiments, the KIR typing is performed by DNA microarray. In some embodiments, the KIR typing is performed by ELISA. In some embodiments, the KIR typing is performed by sequencing. In some embodiments, the KIR typing is performed by next generation sequencing (NGS). As a person of ordinary skill in the art would understand, the KIR typing can be performed by any method described herein or otherwise known in the art.

### 3.2. Samples

In some embodiments, methods provided herein include obtaining a sample from the subject. The sample used in the methods provided herein includes body fluids from a subject. Non-limiting examples of body fluids include blood (e.g., peripheral whole blood, peripheral blood), blood plasma, bone marrow, amniotic fluid, aqueous humor, bile, lymph, menses, serum, urine, cerebrospinal fluid surrounding the brain and the spinal cord, synovial fluid surrounding bone joints.

In one embodiment, the sample is a bone marrow sample. Procedures to obtain a bone marrow sample are well known in the art, including but not limited to bone marrow biopsy and bone marrow aspiration. Bone marrow has a fluid portion and a more solid portion. In bone marrow biopsy, a sample of the solid portion is taken. In bone marrow aspiration, a sample of the fluid portion is taken. Bone marrow biopsy and bone marrow aspiration can be done at the same time and referred to as a bone marrow exam.

In some embodiments, the sample is a blood sample. The blood sample can be obtained using conventional techniques as described in, e.g. Innis et al, editors, PCR Protocols (Academic Press, 1990). White blood cells can be separated from blood samples using convention techniques or commercially available kits, e.g. RosetteSep kit (Stein Cell Technologies, Vancouver, Canada). Sub-populations of white blood cells, e.g. mononuclear cells, NK cells, B cells, T cells, monocytes, granulocytes or lymphocytes, can be further isolated using conventional techniques, e.g. magnetically activated cell sorting (MACS) (Miltenyi Biotec, Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, 0.4, 0.5, 0.6, 0.7, 0.8, 0.9, 1.0, 1.5, 2.0, 2.5, 3.0, 3.5, 4.0, 4.5, 5.0, 6.0, 7.0, 8.0, 9.0 or 10.0 mL.

In some embodiments, methods provided herein include obtaining a sample from the subject. In some embodiments, the sample is a tumor sample. In some embodiments, the sample used in the present methods includes a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In some embodiments, the sample used in the methods provided herein includes a plurality of cells. Such cells can include any type of cells, e.g., stem cells, blood cells (e.g., PBMCs), lymphocytes, NK cells, B cells, T cells, monocytes, granulocytes, immune cells, or tumor or cancer cells. Specific cell populations can be obtained using a combination of commercially available antibodies (e.g., Quest Diagnostic (San Juan Capistrano, Calif.); Dako (Denmark)).

Samples can be analyzed at a time during an active phase of a cancer (e.g., lymphoma, MDS, or leukemia), or when the cancer is inactive. In some embodiments, more than one sample from a subject can be obtained.

In some embodiments, the sample used in the methods provided herein is from a diseased tissue, e.g., from an individual having cancer (e.g., lymphoma, MDS, or leukemia). In certain embodiments. In some embodiments, the cells can be obtained from the tumor or cancer cells or a tumor tissue, such as a tumor biopsy or a tumor explants. In some embodiments, the number of cells used in the methods provided herein can range from a single cell to about 10⁹ cells. In some embodiments, the number of cells used in the methods provided herein is about 1×10⁴, 5×10⁴, 1×10⁵, 5×10⁵, 1×10⁶, 5×10⁶, 1×10⁷, 5×10⁷, 1×10⁸, or 5×10⁸.

In one embodiment, the sample used in the methods provided herein is obtained from the subject prior to the subject receiving a treatment for the disease or disorder. In another embodiment, the sample is obtained from the subject during the subject receiving a treatment for the disease or disorder. In another embodiment, the sample is obtained from the subject after the subject receiving a treatment for the disease or disorder. In various embodiments, the treatment includes administering an FTI to the subject.

The number and type of cells collected from a subject can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In some embodiments, subsets of cells are used in the methods provided herein. Methods to sort and isolate specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc.

The sample can be a whole blood sample, a bone marrow sample, a partially purified blood sample, or PBMC. The sample can be a tissue biopsy or a tumor biopsy. In some embodiments, the sample is a bone marrow sample from a cancer patient. In some embodiments, the sample is PBMCs from a cancer patient.

Methods of obtaining a sample from a subject and methods to prepare the sample for determining the mutation status of a gene or protein are well known in the art.

### 3.3 Cancers

Provided herein are methods for treating a cancer in a subject with an FTI, and methods for selecting cancer patients for an FTI treatment, based on the presence of a mutation in a member of the KIR family (e.g., KIR2DL1, KIR2DL3, KIR2DL4, KIR3DL1, and/or KIR3DL2). Provided herein are also methods for treating a premalignant condition in a subject with an FTI, and methods for selecting patients with a premalignant condition for an FTI treatment, based on the presence of a mutation in a member of the KIR family (e.g., KIR2DL1, KIR2DL3, KIR2DL4, KIR3DL1, and/or KIR3DL2).

In some embodiments, the methods for treating cancer in a subject include KIR2DL1, KIR2DL3, KIR2DL4, KIR3DL1, and/or KIR3DL2-typing the subject, and administering a therapeutically effective amount of tipifarnib to the subject, wherein the subject is a carrier of a mutation in KIR2DL1, KIR2DL3, KIR2DL4, KIR3DL1, and/or KIR3DL2.

In some embodiments, the methods for treating cancer in a subject include KIR2DL1, KIR2DL3, KIR2DL4, KIR3DL1, and/or KIR3DL2-typing the cancer in the subject, and administering a therapeutically effective amount of tipifarnib to the subject, wherein the cancer has a mutation in KIR2DL1, KIR2DL3, KIR2DL4, KIR3DL1, and/or KIR3DL2.

In some embodiments, provided herein are methods for treating a hematological or hematopoietic cancer in a subject with an FTI or selecting cancer patients for an FTI treatment based on the presence of a mutation in a member of the KIR family (e.g., KIR2DL1, KIR2DL3, KIR2DL4, KIR3DL1, and/or KIR3DL2). Hematologic cancers are cancers of the blood or bone marrow. Examples of hematological (or hematogenous) cancers include myeloproliferative neoplasm (MPN), myelodysplastic syndrome (MDS), leukemia, and lymphoma. In some embodiments, the cancer is acute myeloid leukemia (AML), natural killer cell lymphoma (NK lymphoma), natural killer cell leukemia (NK leukemia), cutaneous T-Cell lymphoma (CTCL), juvenile myelomonocytic leukemia (JMML), peripheral T-cell lymphoma (PTCL), angioimmunoblastic T-cell lymphoma (AITL), T-cell lymphoma, chronic myeloid leukemia (CML) or chronic myelomonocytic leukemia (CMML). In some embodiments, the cancer is CMML. In some embodiments, the cancer is JMML. In some embodiments, the hematological or hematopoietic cancer is HPV negative.

Hematological cancers include leukemias, including acute leukemias (such as acute lymphocytic leukemia, acute myelocytic leukemia, acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia, chronic myeloic leukemia, and chronic lymphocytic leukemia), chronic myelomonocytic leukemia, juvenile myelomonocytic leukemia, polycythemia vera, NK cell leukemia, lymphoma, NK cell lymphoma, Hodgkin's disease, non-Hodgkin's lymphoma (indolent and high grade forms), multiple myeloma, peripheral T-cell lymphomas, cutaneous T-Cell lymphoma, Waldenstrom's macroglobulinemia, heavy chain disease, myeiodysplastic syndrome, agnogenic myeloid metaplasia, familial erythrophagocytic lymphohistiocytosis, hairy cell leukemia and myelodysplasia.

In some embodiments, the hematopoietic cancer to be treated by methods provided herein can be lymphoma, T-cell lymphoma, PTCL, AITL, CTCL, relapsed or refractory PTCL, PTCL-NOS, relapsed or refractory AITL, AITL-NOS, ALCL-ALK positive, ALCL-ALK negative, enteropathy-associated T-cell lymphoma, NK lymphoma, extranodal natural killer cell (NK) T-cell lymphoma—nasal type, hepatosplenic T-cell lymphoma, subcutaneous panniculitis-like T-cell lymphoma, EBV associated lymphoma, leukemia, NK leukemia, AML, T-ALL, CML, MDS, MPN, CMML, or JMML. In some embodiments, the hematopoietic cancer is a MDS. The MDS patient can have very low risk MDS, low risk MDS, intermediate risk MDS, or high risk MDS. In some embodiments, the patient is a lower risk MDS patient, which can have a very low risk MDS, low risk MDS, intermediate risk MDS. In some embodiments, the hematopoietic cancer is CMML. The CMML can be low risk CMML, intermediate risk CMML, or high risk CMML. The CMML can be myelodysplastic CMML or myeloproliferative CMML. In some embodiments, the CMML is a KIR-mutant CMML. In some embodiments, the CMML is NRAS/KRAS wild type CMML. In some embodiments, the hematopoietic cancer is NK lymphoma. In some embodiments, the hematopoietic cancer is NK leukemia. In some embodiments, the hematopoietic cancer is CTCL. In some embodiments, the hematopoietic cancer is PTCL. In some embodiments, the PTCL is refractory or relapsed PTCL.

In some embodiments, provided herein are methods for treating MDS in a subject with an FTI or selecting MDS patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant MDS in a subject by administering a therapeutically effective amount of the FTI to the subject. In some embodiments, the FTI is tipifarnib.

MDS refers to a diverse group of hematopoietic stem cell disorders. MDS can be characterized by a cellular marrow with impaired morphology and maturation (dysmyelopoiesis), ineffective blood cell production, or hematopoiesis, leading to low blood cell counts, or cytopenias (including anemia, leukopenia, and thrombocytopenia), and high risk of progression to acute myeloid leukemia, resulting from ineffective blood cell production. See The Merck Manual 953 (17th ed. 1999) and List et al., 1990, J Clin. Oncol. 8:1424.

MDS can be divided into a number of subtypes depending on at least 1) whether increased numbers of blast cells are present in bone marrow or blood, and what percentage of the marrow or blood is made up of these blasts; 2) whether the marrow shows abnormal growth (dysplasia) in only one type of blood cell (unilineage dysplasia) or in more than one type of blood cell (multilineage dysplasia); and 3) whether there are chromosome abnormalities in marrow cells and, if so, which type or types of abnormalities. MDS can also categorized based on the surface markers of the cancer cells. According to the World Health Organization, MDS subtypes include refractory cytopenia with unilineage dysplasia (RCUD), also known as refractory anemia, refractory neutropenia, or refractory thrombocytopenia; refractory anemia with ring sideroblasts (RARS); refractory cytopenia with multilineage dysplasia (RCMD), which includes RCMD-RS if multilineage dysplasia and ring sideroblasts both are present; refractory anemia with excess blasts-1 (RAEB-1) and refractory anemia with excess blasts-2 (RAEB-2) (These subtypes mean that the patients have at least 5 percent (RAEB-1) or at least 10 percent (RAEB-2) but less than 20 percent blasts in their marrow); MDS associated with isolated abnormality of chromosome 5 [del(5q)]; and unclassifiable MDS (MDS-U).

As a group of hematopoietic stem cell malignancies with significant morbidity and mortality, MDS is a highly heterogeneous disease, and the severity of symptoms and disease progression can vary widely among patients. The current standard clinical tool to evaluate risk stratification and treatment options is the revised International Prognostic Scoring System, or IPSS-R. The IPSS-R differentiates patients into five risk groups (Very Low, Low, Intermediate, High, Very High) based on evaluation of cytogenetics, percentage of blasts (undifferentiated blood cells) in the bone marrow, hemoglobin levels, and platelet and neutrophil counts. The WHO also suggested stratifying MDS patients by a del (5q) abnormality.

According to the ACS, the annual incidence of MDS is approximately 13,000 patients in the United States, the majority of which are 60 years of age or older. The estimated prevalence is over 60,000 patients in the United States. Approximately 75% of patients fall into the IPSS-R risk categories of Very Low, Low, and Intermediate, or collectively known as lower risk MDS.

The initial hematopoietic stem cell injury can be from causes such as, but not limited to, cytotoxic chemotherapy, radiation, virus, chemical exposure, and genetic predisposition. A clonal mutation predominates over bone marrow, suppressing healthy stem cells. In the early stages of MDS, the main cause of cytopenias is increased programmed cell death (apoptosis). As the disease progresses and converts into leukemia, gene mutation rarely occurs and a proliferation of leukemic cells overwhelms the healthy marrow. The disease course differs, with some cases behaving as an indolent disease and others behaving aggressively with a very short clinical course that converts into an acute form of leukemia.

An international group of hematologists, the French-American-British (FAB) Cooperative Group, classified MDS disorders into five subgroups, differentiating them from AML. The Merck Manual 954 (17th ed. 1999); Bennett J. M., et al., Ann. Intern. Med. 1985 October, 103(4): 620-5; and Besa E. C., Med. Clin. North Am. 1992 May, 76(3): 599-617. An underlying trilineage dysplastic change in the bone marrow cells of the patients is found in all subtypes.

There are two subgroups of refractory anemia characterized by five percent or less myeloblasts in bone marrow: (1) refractory anemia (RA) and; (2) RA with ringed sideroblasts (RARS), defined morphologically as having 15% erythroid cells with abnormal ringed sideroblasts, reflecting an abnormal iron accumulation in the mitochondria. Both have a prolonged clinical course and low incidence of progression to acute leukemia. Besa E. C., Med. Clin. North Am. 1992 May, 76(3): 599-617.

There are two subgroups of refractory anemias with greater than five percent mycloblasts: (1) RA with excess blasts (RAEB), defined as 6-20% myeloblasts, and (2) RAEB in transformation (RAEB-T), with 21-30% myeloblasts. The higher the percentage of myeloblasts, the shorter the clinical course and the closer the disease is to acute myelogenous leukemia. Patient transition from early to more advanced stages indicates that these subtypes are merely stages of disease rather than distinct entities. Elderly patients with MDS with trilineage dysplasia and greater than 30% myeloblasts who progress to acute leukemia are often considered to have a poor prognosis because their response rate to chemotherapy is lower than de novo acute myeloid leukemia patients. The fifth type of MDS, the most difficult to classify, is CMML. This subtype can have any percentage of myeloblasts but presents with a monocytosis of 1000/dL or more. It may be associated with splenomegaly. This subtype overlaps with a myeloproliferative disorder and may have an intermediate clinical course. It is differentiated from the classic CML that is characterized by a negative Ph chromosome.

MDS is primarily a disease of elderly people, with the median onset in the seventh decade of life. The median age of these patients is 65 years, with ages ranging from the early third decade of life to as old as 80 years or older. The syndrome may occur in any age group, including the pediatric population. Patients who survive malignancy treatment with alkylating agents, with or without radiotherapy, have a high incidence of developing MDS or secondary acute leukemia. About 60-70% of patients do not have an obvious exposure or cause for MDS, and are classified as primary MDS patients.

The treatment of MDS is based on the stage and the mechanism of the disease that predominates the particular phase of the disease process. Bone marrow transplantation has been used in patients with poor prognosis or late-stage MDS. Epstein and Slease, 1985, Surg. Ann. 17:125. An alternative approach to therapy for MDS is the use of hematopoietic growth factors or cytokines to stimulate blood cell development in a recipient. Dexter, 1987, J. Cell Sci. 88:1; Moore, 1991, Annu. Rev. Immunol. 9:159; and Besa E. C., Med. Clin. North Am. 1992 May, 76(3): 599-617. The treatment of MDS using immunomodulatory compounds is described in U.S. Pat. No. 7,189,740, the entirety of which is hereby incorporated by reference.

Therapeutic options fall into three categories including supportive care, low intensity and high intensity therapy. Supportive care includes the use red blood cell and platelet transfusions and hematopoietic cytokines such as erythropoiesis stimulating agents or colony stimulating factors to improve blood counts. Low intensity therapies include hypomethylating agents such as azacytidine (Vidaza®) and decitabine (Dacogen®), biological response modifiers such as lenalidomide (Revlimid®), and immunosuppressive treatments such as cyclosporine A or antithymocyte globulin. High intensity therapies include chemotherapeutic agents such as idarubicin, azacytidine, fludarabine and topotecan, and hematopoietic stem cell transplants, or HSCT.

National Comprehensive Cancer Network, or NCCN, guidelines recommend that lower risk patients (IPSS-R groups Very Low, Low, Intermediate) receive supportive care or low intensity therapies with the major therapeutic goal of hematologic improvement, or HI. NCCN guidelines recommend that higher risk patients (IPSS-R groups High, Very High) receive more aggressive treatment with high intensity therapies. In some cases, high risk patients are unable to tolerate chemotherapy, and may elect lower intensity regimens. Despite currently available treatments, a substantial portion of MDS patients lack effective therapies and NCCN guidelines recommend clinical trials as additional therapeutic options. Treatment of MDS remains a significant unmet need requiring the development of novel therapies.

In some embodiments, provided herein are methods for treating MPN in a subject with an FTI or selecting MPN patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant MPN in a subject by administering a therapeutically effective amount of the FTI to the subject. In some embodiments, the FTI is tipifarnib.

MPN is a group of diseases that affect blood-cell formation. In all forms of MPN, stem cells in the bone marrow develop genetic defects (called acquired defects) that cause them to grow and survive abnormally. This results in unusually high numbers of blood cells in the bone marrow (hypercellular marrow) and in the bloodstream. Sometimes in MPN, the abnormal stem cells cause scarring in the marrow, called myelofibrosis. Myelofibrosis can lead to low levels of blood cells, especially low levels of red blood cells (anemia). In MPN, the abnormal stem cells can also grow in the spleen, causing the spleen to enlarge (splenomegaly), and in other sites outside the marrow, causing enlargement of other organs.

There are several types of chronic MPN, based on the cells affected. Three classic types of MPN include polycythemia vera (PV), in which there are too many RBCs; essential thrombocythemia (ET), in which there are too many platelets; primary myelofibrosis (PMF), in which fibers and blasts (abnormal stem cells) build up in the bone marrow. Other types of MPN include: chronic myeloid leukemia, in which there are too many white blood cells; chronic neutrophilic leukemia, in which there are too many neutrophils; chronic eosinophilic leukemia, not otherwise specified, in which there are too many eosinophils (hypereosinophilia); mastocytosis, also called mast cell disease, in which there are too many mast cells, which are a type of immune system cell found in tissues, like skin and digestive organs, rather than in the bloodstream; myeloid and lymphoid neoplasms with eosinophilia and abnormalities of the PDGFRA, PDGFRB, and FGFR1 genes; and other unclassifiable myeloproliferative neoplasms.

In some embodiments, provided herein are methods for treating hematological cancer (e.g., leukemia, lymphoma, myeloproliferative neoplasm (MPN), or myelodysplastic syndrome (MDS)) or a solid tumor in a subject with an FTI or selecting the patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant hematological cancer (e.g., leukemia, lymphoma, myeloproliferative neoplasm (MPN), or myelodysplastic syndrome (MDS)) or a solid tumor in a subject by administering a therapeutically effective amount of the FTI to the subject. In some embodiments, the FTI is tipifarnib. In some embodiments, the KIR-mutant hematological cancer is a leukemia that has a mutation in a member of the KIR family (e.g., KIR2DL1, KIR2DL3, KIR2DL4, KIR3DL1, and/or KIR3DL2). In some embodiments, the KIR-mutant hematological cancer is lymphoma (e.g., T-cell lymphoma) that has a mutation in KIR2DL1, KIR2DL3, KIR2DL4, KIR3DL1, and/or KIR3DL2.

In some embodiments, provided herein are methods for treating PTCL (e.g., PTCL-NOS or AITL) in a subject with an FTI or selecting PTCL patients (e.g., PTCL-NOS or AITL patients) for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant PTCL in a subject by administering a therapeutically effective amount of the FTI to the subject. In some embodiments, the FTI is tipifarnib. In some embodiments, the KIR-mutant PTCL is a PTCL-NOS that has a mutation in a member of the KIR family (e.g., KIR2DL1, KIR2DL3, KIR2DL4, KIR3DL1, and/or KIR3DL2). In some embodiments, the KIR-mutant PTCL is an AITL that has a mutation in KIR2DL1, KIR2DL3, KIR2DL4, KIR3DL1, and/or KIR3DL2.

In some embodiments, provided herein are methods for predicting responsiveness of a CMML patient to an FTI treatment (e.g., tipifarnib), methods for CMML patient population selection for an FTI treatment, and methods for treating CMML in a subject with a therapeutically effective amount of an FTI, based on the mutation status of a member of the KIR family (e.g., KIR2DL1, KIR2DL3, KIR2DL4, KIR3DL1, and/or KIR3DL2) in a sample from the patient. In some embodiments, provided herein is a method of treating CMML in a subject based on the mutation status of KIR2DL1, KIR2DL3, KIR2DL4, KIR3DL1, and/or KIR3DL2. The method provided herein includes (a) determining the presence or absence of a mutation in KIR2DL1, KIR2DL3, KIR2DL4, KIR3DL1, and/or KIR3DL2 in a sample from the subject, and subsequently (b) administering a therapeutically effective amount of an FTI (e.g., tipifarnib) to said subject if said sample is determined to have a mutation in KIR2DL1, KIR2DL3, KIR2DL4, KIR3DL1, and/or KIR3DL2.

In some embodiments, provided herein are methods for treating leukemia in a subject with an FTI or selecting leukemia patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant leukemia in a subject by administering a therapeutically effective amount of the FTI to the subject. In some embodiments, the FTI is tipifarnib.

Leukemia refers to malignant neoplasms of the blood-forming tissues. Various forms of leukemias are described, for example, in U.S. Pat. No. 7,393,862 and U.S. provisional patent application No. 60/380,842, filed May 17, 2002, the entireties of which are incorporated herein by reference. Although viruses reportedly cause several forms of leukemia in animals, causes of leukemia in humans are to a large extent unknown. The Merck Manual, 944-952 (17th ed. 1999). Transformation to malignancy typically occurs in a single cell through two or more steps with subsequent proliferation and clonal expansion. In some leukemias, specific chromosomal translocations have been identified with consistent leukemic cell morphology and special clinical features (e.g., translocations of 9 and 22 in chronic myelocytic leukemia, and of 15 and 17 in acute promyelocytic leukemia). Acute leukemias are predominantly undifferentiated cell populations and chronic leukemias more mature cell forms.

Acute leukemias are divided into lymphoblastic (ALL) and non-lymphoblastic (ANLL) types. The Merck Manual, 946-949 (17^{th} ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease which is established by laboratory findings and bone marrow examination. ANLL, also known as acute myelogenous leukemia or AML, occurs at all ages and is the more common acute leukemia among adults; it is the form usually associated with irradiation as a causative agent. In some embodiments, provided herein are methods for treating a AML patient with an FTI, or methods for selecting patients for FTI treatment.

In some embodiments, provided herein are methods for treating AML in a subject with an FTI or selecting AML patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant AML in a subject by administering a therapeutically effective amount of the FTI to the subject. In some embodiments, the FTI is tipifarnib.

Standard procedures treat AML patients usually include 2 chemotherapy (chemo) phases: remission induction (or induction) and consolidation (post-remission therapy). The first part of treatment (remission induction) is aimed at getting rid of as many leukemia cells as possible. The intensity of the treatment can depend on a person's age and health. Intensive chemotherapy is often given to people under the age of 60. Some older patients in good health can benefit from similar or slightly less intensive treatment. People who are much older or are in poor health are not suitable for intensive chemotherapies.

In some embodiments, the AML patient is post-remission induction. In some embodiments, the AML patient post-transplantation. In some embodiments, the AML patient is over age 60 or otherwise unfit for remission induction. In some embodiments, the AML patient is over age 65, 70, or 75. In some embodiments, the AML patient is refractory to standard chemotherapy. In some embodiments, the AML patient is a relapsed patient.

In younger patients, such as those under 60, induction often involves treatment with 2 chemo drugs, cytarabine (ara-C) and an anthracycline drug such as daunorubicin (daunomycin) or idarubicin. Sometimes a third drug, cladribine (Leustatin, 2-CdA), is given as well. The chemo is usually given in the hospital and lasts about a week. In rare cases where the leukemia has spread to the brain or spinal cord, chemo may also be given into the cerebrospinal fluid (CSF). Radiation therapy might be used as well.

Induction is considered successful if remission is achieved. However, the AML in some patients can be refractory to induction. In patients who respond to induction, further treatment is then given to try to destroy remaining leukemia cells and help prevent a relapse, which is called consolidation. For younger patients, the main options for consolidation therapy are: several cycles of high-dose cytarabine (ara-C) chemo (sometimes known as HiDAC); allogeneic (donor) stem cell transplant; and autologous stem cell transplant.

Chronic leukemias are described as being lymphocytic (CLL) or myelocytic (CML). The Merck Manual, 949-952 (17^{th} ed. 1999). CLL is characterized by the appearance of mature lymphocytes in blood, bone marrow, and lymphoid organs. The hallmark of CLL is sustained, absolute lymphocytosis (>5,000/μL) and an increase of lymphocytes in the bone marrow. Most CLL patients also have clonal expansion of lymphocytes with B-cell characteristics. CLL is a disease of middle or old age. In CML, the characteristic feature is the predominance of granulocytic cells of all stages of differentiation in blood, bone marrow, liver, spleen, and other organs. In the symptomatic patient at diagnosis, the total white blood cell (WBC) count is usually about 200,000/μL, but may reach 1,000,000/μL. CML is relatively easy to diagnose because of the presence of the Philadelphia chromosome. Bone marrow stromal cells are well known to support CLL disease progression and resistance to chemotherapy. Disrupting the interactions between CLL cells and stromal cells is an additional target of CLL chemotherapy.

Additionally, other forms of CLL include prolymphocytic leukemia (PLL), Large granular lymphocyte (LGL) leukemia, Hairy cell leukemia (HCL). The cancer cells in PLL are similar to normal cells called prolymphocytes—immature forms of B lymphocytes (B-PLL) or T lymphocytes (T-PLL). Both B-PLL and T-PLL tend to be more aggressive than the usual type of CLL. The cancer cells of LGL are large and have features of either T cells or NK cells. Most LGL leukemias are slow-growing, but a small number are more aggressive. HCL is another cancer of lymphocytes that tends to progress slowly, and accounts for about 2% of all leukemias. The cancer cells are a type of B lymphocyte but are different from those seen in CLL.

In some embodiments, provided herein are methods for treating chronic leukemia (e.g., CML) in a subject with an FTI or selecting a chronic leukemia patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant chronic leukemia (e.g., CML) in a subject by administering a therapeutically effective amount of the FTI to the subject. In some embodiments, the FTI is tipifarnib.

Juvenile myelomonocytic leukemia (JMML) is a serious chronic leukemia that affects children mostly aged 4 and under. The average age of patients at diagnosis is 2 years old. The World Health Organization has categorized JMML as a mixed myelodysplastic and myeloproliferative disorder. The JMML encompasses diagnoses formerly referred to as Juvenile Chronic Myeloid Leukemia (JCML), Chronic Myelomonocytic Leukemia of Infancy, and Infantile Monosomy 7 Syndrome.

In some embodiments, provided herein are methods for treating JMML in a subject with an FTI or selecting JMML patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant JMML in a subject by administering a therapeutically effective amount of the FTI to the subject. In some embodiments, the FTI is tipifarnib.

In some embodiments, provided herein are methods for treating a lymphoma in a subject with an FTI or selecting lymphoma patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant lymphoma in a subject by administering a therapeutically effective amount of the FTI to the subject. In some embodiments, the FTI is tipifarnib.

Lymphoma refers to cancers that originate in the lymphatic system. Lymphoma is characterized by malignant neoplasms of lymphocytes—B lymphocytes (B cell lymphoma), T lymphocytes (T-cell lymphoma), and natural killer cells (NK cell lymphoma). Lymphoma generally starts in lymph nodes or collections of lymphatic tissue in organs including, but not limited to, the stomach or intestines. Lymphoma may involve the marrow and the blood in some cases. Lymphoma may spread from one site to other parts of the body.

The treatments of various forms of lymphomas are described, for example, in U.S. Pat. No. 7,468,363, the entirety of which is incorporated herein by reference. Such lymphomas include, but are not limited to, Hodgkin's lymphoma, non-Hodgkin's lymphoma, cutaneous B-cell lymphoma, activated B-cell lymphoma, Diffuse Large B-Cell Lymphoma (DLBCL), mantle cell lymphoma (MCL), follicular lymphoma (FL; including but not limited to FL grade I, FL grade II), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma (CTCL) and mantle zone lymphoma and low grade follicular lymphoma.

Non-Hodgkin's lymphoma (NHL) is the fifth most common cancer for both men and women in the United States, with an estimated 63,190 new cases and 18,660 deaths in 2007. Jemal A, et al., CA Cancer J Clin 2007; 57(1):43-66. The probability of developing NHL increases with age and the incidence of NHL in the elderly has been steadily increasing in the past decade, causing concern with the aging trend of the U.S. population. Id. Clarke C A, et al., Cancer 2002; 94(7):2015-2023.

DLBCL accounts for approximately one-third of non-Hodgkin's lymphomas. While some DLBCL patients are cured with traditional chemotherapy, the remainders die from the disease. Anticancer drugs cause rapid and persistent depletion of lymphocytes, possibly by direct apoptosis induction in mature T and B cells. See K. Stahnke. et al., Blood 2001, 98:3066-3073. Absolute lymphocyte count (ALC) has been shown to be a prognostic factor in follicular non-Hodgkin's lymphoma and recent results have suggested that ALC at diagnosis is an important prognostic factor in DLBCL.

DLBCL can be divided into distinct molecular subtypes according to their gene profiling patterns: germinal-center B-cell-like DLBCL (GCB-DLBCL), activated B-cell-like DLBCL (ABC-DLBCL), and primary mediastinal B-cell lymphoma (PMBL) or unclassified type. These subtypes are characterized by distinct differences in survival, chemo-responsiveness, and signaling pathway dependence, particularly the NF-κB pathway. See D. Kim et al., Journal of Clinical Oncology, 2007 ASCO Annual Meeting Proceedings Part I. Vol 25, No. 18S (June 20 Supplement), 2007: 8082. See Bea S, et al., Blood 2005; 106: 3183-90; Ngo V. N. et al., Nature 2011; 470: 115-9. Such differences have prompted the search for more effective and subtype-specific treatment strategies in DLBCL. In addition to the acute and chronic categorization, neoplasms are also categorized based upon the cells giving rise to such disorder into precursor or peripheral. See e.g., U.S. patent Publication No. 2008/0051379, the disclosure of which is incorporated herein by reference in its entirety. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, Diffuse large B-cell lymphoma (DLBCL) and Burkitt lymphoma. In over 95 percent of CLL cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype. Notwithstanding these classifications, however, the pathological impairment of normal hematopoiesis is the hallmark of all leukemias.

PTCL consists of a group of rare and usually aggressive (fast-growing) NHLs that develop from mature T-cells. PTCLs collectively account for about 4 to 10 percent of all NHL cases, corresponding to an annual incidence of 2,800-7,200 patients per year in the United States. By some estimates, the incidence of PTCL is growing significantly, and the increasing incidence may be driven by an aging population. PTCLs are sub-classified into various subtypes, each of which are typically considered to be separate diseases based on their distinct clinical differences. Most of these subtypes are rare; the three most common subtypes of PTCL not otherwise specified, anaplastic large-cell lymphoma, or ALCL, and angioimmunoblastic T-cell lymphoma, that collectively account for approximately 70 percent of all PTCLs in the United States. ALCL can be cutaneous ALCL or systemic ALCL.

For most PTCL subtypes, the frontline treatment regimen is typically combination chemotherapy, such as CHOP (cyclophosphamide, doxorubicin, vincristine, prednisone), EPOCH (etoposide, vincristine, doxorubicin, cyclophosphamide, prednisone), or other multi-drug regimens. Patients who relapse or are refractory to frontline treatments are typically treated with gemcitabine in combination with other chemotherapies, including vinorelbine (Navelbine®) and doxorubicin (Doxil®) in a regimen called GND, or other chemotherapy regimens such as DHAP (dexamethasone, cytarabine, cisplatin) or ESHAP (etoposide, methylprednisolone, cytarabine, and cisplatin).

Because most patients with PTCL will relapse, some oncologists recommend giving high-dose chemotherapy followed by an autologous stem cell transplant to some patients who had a good response to their initial chemotherapy. Recent, non-cytotoxic therapies that have been approved for relapsed or refractory PTCL, such as pralatrexate (Folotyn®), romidepsin (Istodax®) and belinostat (Beleodaq®), are associated with relatively low objective response rates (25-27% overall response rate, or ORR) and relatively short durations of response (8.2-9.4 months). Accordingly, the treatment of relapsed/refractory PTCL remains a significant unmet medical need.

In some embodiments, provided herein are methods for treating multiple myeloma in a subject with an FTI or selecting multiple myeloma patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant multiple myeloma in a subject by administering a therapeutically effective amount of the FTI to the subject. In some embodiments, the FTI is tipifarnib.

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin. M-protein, short for monoclonal protein, also known as paraprotein, is a particularly abnormal protein produced by the myeloma plasma cells and can be found in the blood or urine of almost all patients with multiple myeloma.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

Bone marrow stromal cells are well known to support multiple myeloma disease progression and resistance to chemotherapy. Disrupting the interactions between multiple myeloma cells and stromal cells is an additional target of multiple myeloma chemotherapy.

In some embodiments, provided herein are methods for treating a solid tumor with an FTI based on the presence of a mutation in a member of the KIR family (such as KIR2DL1, KIR2DL3, KIR2DL4, KIR3DL1, and/or KIR3DL2). In some embodiments, provided herein are methods selecting multiple solid tumor patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant solid tumor in a subject by administering a therapeutically effective amount of the FTI to the subject. In some embodiments, the FTI is tipifarnib.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). The solid tumor to be treated with the methods of the invention can be sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, meduloblastoma, Schwannoma craniopharyogioma, ependymoma, pineaioma, hemangioblastoma, acoustic neuroma, oligodendroglioma, menangioma, neuroblastoma, retinoblastoma and brain metastases). In some embodiments, the FTI is tipifarnib.

In some embodiments, provided herein are methods for treating a solid tumor with an FTI based on the presence of a mutation in a member of the KIR family (such as KIR2DL1, KIR2DL3, KIR2DL4, KIR3DL1, and/or KIR3DL2), wherein the solid tumor is malignant melanoma, adrenal carcinoma, breast carcinoma, renal cell cancer, carcinoma of the pancreas, non-small-cell lung carcinoma (NSCLC) or carcinoma of unknown primary. In some embodiments, the FTI is tipifarnib. Drugs commonly administered to patients with various types or stages of solid tumors include, but are not limited to, celebrex, etoposide, cyclophosphamide, docetaxel, apecitabine, IFN, tamoxifen, IL-2, GM-CSF, or a combination thereof.

In some embodiments, the solid tumor to be treated by methods provided herein can be thyroid cancer, head and neck cancers, urothelial cancers, salivary cancers, cancers of the upper digestive tract, bladder cancer, breast cancer, ovarian cancer, brain cancer, gastric cancer, prostate cancer, lung cancer, colon cancer, skin cancer, liver cancer, and pancreatic cancer. In some embodiments, the bladder cancer to be treated by methods provided herein can be transitional cell carcinoma. In some embodiments, the FTI is tipifarnib.

In some embodiments, the solid tumor to be treated by methods provided herein can be selected from the groups consisting of carcinoma, melanoma, sarcoma, or chronic granulomatous disease.

In some embodiments, the solid tumor to be treated by methods provided herein can be selected from the groups consisting of thyroid cancer, head and neck cancers, or salivary gland cancer. In some embodiments, the solid tumor is thyroid cancer. In some embodiments, the thyroid cancer can be relapsed/recurrent thyroid cancer. In some embodiments, the thyroid cancer can be metastatic thyroid cancer. In some embodiments, the thyroid cancer can be advanced thyroid cancer. In some embodiments, the solid tumor is head and neck squamous cell carcinoma (HNSCC) (e.g., HPV negative HSNCC or HPV positive HSNCC). In some embodiments, the HNSCC can be HPV negative HNSCC. In some embodiments, the HNSCC can be relapsed/recurrent HNSCC. In some embodiments, the HNSCC can be metastatic HNSCC. In some embodiments, the solid tumor is salivary gland cancer. In some embodiments, the salivary gland cancer can be advanced salivary gland cancer. In some embodiments, the salivary gland cancer can be metastatic salivary gland cancer.

In some embodiments, provided herein are methods for treating premalignant conditions in a subject with an FTI or selecting premalignant condition patients for an FTI treatment based on the presence of a mutation in a member of the KIR family (e.g., KIR2DL1, KIR2DL3, KIR2DL4, KIR3DL1, and/or KIR3DL2). In some embodiments, provided herein are methods of treating a KIR-mutant premalignant condition in a subject by administering a therapeutically effective amount of the FTI to the subject. In some embodiments, the FTI is tipifarnib. In some embodiments, the premalignant conditions to be treated by methods provided herein can be actinic cheilitis, Barrett's esophagus, atrophic gastritis, ductal carcinoma in situ, Dyskeratosis congenita, Sideropenic dysphagia, Lichen planus, Oral submucous fibrosis, Solar elastosis, cervical dysplasia, polyps, leukoplakia, erythroplakia, squamous intraepithelial lesion, a pre-malignant disorder, or a pre-malignant immunoproliferative disorder.

In some embodiments, the cancer to be treated by methods provided herein can have a KIR2DL1, KIR2DL3, KIR2DL4, KIR3DL1, and/or KIR3DL2 mutation. In some embodiments, the cancer to be treated by methods provided herein can be a hematologic or hematopoietic cancer with a KIR2DL1, KIR2DL3, KIR2DL4, KIR3DL1, and/or KIR3DL2 mutation. The hematopoietic cancer with a KIR2DL1, KIR2DL3, KIR2DL4, KIR3DL1, and/or KIR3DL2 mutation can be any of the hematologic or hematopoietic cancers described above. In some embodiments, the cancer to be treated by methods provided herein can be a solid tumor with a KIR2DL1, KIR2DL3, KIR2DL4, KIR3DL1, and/or KIR3DL2 mutation. The solid tumor with a KIR2DL1, KIR2DL3, KIR2DL4, KIR3DL1, and/or KIR3DL2 mutation can be any of the solid tumors described above. Methods provided herein or otherwise known in the art can be used to determine the mutation status of a KIR2DL1, KIR2DL3, KIR2DL4, KIR3DL1, and/or KIR3DL2 gene. In some embodiments, the mutation status can be determined an NGS-based assay. In some embodiments, the mutation status can be determined by a qualitative PCR-based assay. In some embodiments, mutation status of a KIR2DL1, KIR2DL3, KIR2DL4, KIR3DL1, and/or KIR3DL2 gene can be determined in the form of a companion diagnostic to the FTI treatment, such as the tipifarnib treatment.

In some embodiments, the treatment of cancer in accordance with the methods described herein achieves at least one, two, three, four or more of the following effects: (i) inhibition of cancer progression, (ii) increase in progression free survival, (iii) increase in tumor-free survival rate of patients; (iv) increase in duration of response to treatment, (v) reduction of tumor growth, (vi) decrease in tumor size (e.g., volume or diameter); (vii) prevention of metastasis, (viii) decrease in metastases (e.g., decrease in the number of metastases); (ix) increase in relapse free survival; (x) alleviation or reduction of one or more symptoms of cancer, and (xi) increase in symptom-free survival.

### 3.4. Exemplary FTIs and Dosages

In some embodiments, provided herein is a method of treating a cancer in a subject with an FTI based on the mutation status of a member of the KIR family (such as KIR2DL1, KIR2DL3, KIR2DL4, KIR3DL1, and/or KIR3DL2). The FTI can be any FTI described herein or otherwise known in the art. In some embodiments, the FTI is selected from the group consisting of tipifarnib, arglabin, perrilyl alcohol, lonafarnib(SCH-66336), L778123, L739749, FTI-277, L744832, CP-609,754, R208176, AZD3409, and BMS-214662. In some embodiments, the FTI is tipifarnib.

In some embodiments, provided herein is a method of treating a hematological or hematopoietic cancer in a subject based on the mutation status of KIR2DL1, KIR2DL3, KIR2DL4, KIR3DL1, and/or KIR3DL2. The method provided herein includes (a) determining the presence or absence of a mutation in KIR2DL1, KIR2DL3, KIR2DL4, KIR3DL1, and/or KIR3DL2 in a sample from the subject, and subsequently (b) administering a therapeutically effective amount of tipifarnib to said subject if said sample is determined to have a mutation in KIR2DL1, KIR2DL3, KIR2DL4, KIR3DL1, and/or KIR3DL2. In some embodiments, the methods include administering the subject with another FTI described herein or otherwise known in the art. In some embodiments, the FTI is selected from the group consisting of tipifarnib, arglabin, perrilyl alcohol, lonafarnib(SCH-66336), L778123, L739749, FTI-277, L744832, CP-609,754, R208176, AZD3409, and BMS-214662.

In some embodiments, provided herein is a method of treating CMML in a subject based on the mutation status of KIR2DL1, KIR2DL3, KIR2DL4, KIR3DL1, and/or KIR3DL2. The method provided herein includes (a) determining the presence or absence of a mutation in KIR2DL1, KIR2DL3, KIR2DL4, KIR3DL1, and/or KIR3DL2 in a sample from the subject, and subsequently (b) administering a therapeutically effective amount of tipifarnib to said subject if said sample is determined to have a mutation in KIR2DL1, KIR2DL3, KIR2DL4, KIR3DL1, and/or KIR3DL2. In some embodiments, the methods include administering the subject with another FTI described herein or otherwise known in the art. In some embodiments, the FTI is selected from the group consisting of tipifarnib, arglabin, perrilyl alcohol, lonafarnib(SCH-66336), L778123, L739749, FTI-277, L744832, CP-609,754, R208176, AZD3409, and BMS-214662.

In some embodiments, the FTI is administered orally, parenterally, rectally, or topically. In some embodiments, the FTI is administered orally. In some embodiments, tipifarnib is administered orally, parenterally, rectally, or topically. In some embodiments, tipifarnib is administered orally.

In some embodiments, the FTI is administered at a dose of 1-1000 mg/kg body weight. In some embodiments, the FTI is administered twice a day. In some embodiments, the FTI is administered at a dose of 200-1200 mg twice a day. In some embodiments, the FTI is administered at a dose of 600 mg twice a day. In some embodiments, the FTI is administered at a dose of 900 mg twice a day. In some embodiments, tipifarnib is administered at a dose of 1-1000 mg/kg body weight. In some embodiments, tipifarnib is administered twice a day. In some embodiments, tipifarnib is administered at a dose of 200-1200 mg twice a day. In some embodiments, tipifarnib is administered at a dose of 300 mg twice a day. In some embodiments, tipifarnib is administered at a dose of 600 mg twice a day. In some embodiments, tipifarnib is administered at a dose of 900 mg twice a day. In some embodiments, tipifarnib is administered at a dose in the range of 200 to 900 mg twice a day.

In some embodiments, the FTI is administered at a dose of 1-1000 mg/kg body weight. In some embodiments, the FTI is administered twice a day. In some embodiments, the FTI is administered at a dose of 200-1200 mg twice a day. In some embodiments, the FTI is administered at a dose of 300 mg twice a day. In some embodiments, the FTI is administered at a dose of 600 mg twice a day. In some embodiments, the FTI is administered at a dose of 900 mg twice a day. In some embodiments, the FTI is administered at a dose in the range of 200 to 900 mg twice a day. In some embodiments, tipifarnib is administered in treatment cycles. In some embodiments, tipifarnib is administered in alternative weeks. In some embodiments, tipifarnib is administered on days 1-7 and 15-21 of a 28-day treatment cycle. In some embodiments, tipifarnib is administered orally at a dose of 900 mg twice a day on days 1-7 and 15-21 of a 28-day treatment cycle.

In some embodiments, the FTI is administered in treatment cycles. In some embodiments, the FTI is administered in alternative weeks. In some embodiments, the FTI is administered on days 1-7 and 15-21 of a 28-day treatment cycle. In some embodiments, the FTI is administered orally at a dose of 900 mg twice a day on days 1-7 and 15-21 of a 28-day treatment cycle. In some embodiments, the FTI is administered on days 1-21 of a 28-day treatment cycle (e.g, orally at a dose of 900 mg twice a day). In some embodiments, the FTI is administered on days 1-7 of a 28-day treatment cycle (e.g, orally at a dose of 900 mg twice a day). In some embodiments, tipifarnib is administered in treatment cycles. In some embodiments, tipifarnib is administered in alternative weeks. In some embodiments, tipifarnib is administered on days 1-7 and 15-21 of a 28-day treatment cycle. In some embodiments, tipifarnib is administered orally at a dose of 900 mg twice a day on days 1-7 and 15-21 of a 28-day treatment cycle. In some embodiments, tipifarnib is administered on days 1-21 of a 28-day treatment cycle (e.g, orally at a dose of 900 mg twice a day). In some embodiments, tipifarnib is administered on days 1-7 of a 28-day treatment cycle (e.g, orally at a dose of 900 mg twice a day).

In some embodiments, the FTI is administered for at least 3 cycles. In some embodiments, the FTI is administered for at least 6 cycles. In some embodiments, the FTI is administered for up to 12 cycles. In some embodiments, the FTI is administered orally at a dose of 900 mg twice a day on days 1-7 and 15-21 of a 28-day treatment cycle for at least three cycles. In some embodiments, tipifarnib is administered for at least 3 cycles. In some embodiments, tipifarnib is administered for at least 6 cycles. In some embodiments, tipifarnib is administered for up to 12 cycles. In some embodiments, tipifarnib is administered orally at a dose of 900 mg twice a day on days 1-7 and 15-21 of a 28-day treatment cycle for at least three cycles.

In some embodiments, the FTI is administered for at least 3 cycles. In some embodiments, the FTI is administered for at least 6 cycles. In some embodiments, the FTI is administered for up to 12 cycles. In some embodiments, the FTI is administered orally at a dose in the range of 200 mg to 900 mg twice a day on days 1-21 of a 28-day treatment cycle for at least three cycles. In some embodiments, tipifarnib is administered for at least 3 cycles. In some embodiments, tipifarnib is administered for at least 6 cycles. In some embodiments, tipifarnib is administered for up to 12 cycles. In some embodiments, tipifarnib is administered orally at a dose in the range of 200 mg to 900 mg twice a day on days 1-21 of a 28-day treatment cycle for at least three cycles.

In some embodiments, the FTI is administered for at least 3 cycles. In some embodiments, the FTI is administered for at least 6 cycles. In some embodiments, the FTI is administered for up to 12 cycles. In some embodiments, the FTI is administered orally at a dose in the range of 200 mg to 900 mg twice a day on days 1-7 of a 28-day treatment cycle for at least three cycles. In some embodiments, tipifarnib is administered for at least 3 cycles. In some embodiments, tipifarnib is administered for at least 6 cycles. In some embodiments, tipifarnib is administered for up to 12 cycles. In some embodiments, tipifarnib is administered orally at a dose in the range of 200 mg to 900 mg twice a day on days 1-7 of a 28-day treatment cycle for at least three cycles.

In some embodiments, provided herein are methods for treating CMML in a subject with a therapeutically effective amount of an tipifarnib, based on the mutation status of KIR2DL1, KIR2DL3, KIR2DL4, KIR3DL1, and/or KIR3DL2 in a sample from the patient. In some embodiments, provided herein is a method of treating CMML in a subject including (a) determining a sample from the subject to have a mutant KIR2DL1, KIR2DL3, KIR2DL4, KIR3DL1, and/or KIR3DL2, and subsequently (b) administering tipifarnib to the subject at a dose in the range of 200 to 900 mg twice a day on days 1-7 and 15-21 of a 28-day treatment cycle.

In some embodiments, provided herein are methods for treating CMML in a subject with a therapeutically effective amount of an tipifarnib, based on the mutation status of KIR in a sample from the patient. In some embodiments, provided herein is a method of treating CMML in a subject including (a) determining a sample from the subject to have a mutant KIR, and subsequently (b) administering tipifarnib to the subject at a dose in the range of 200 to 900 mg twice a day on days 1-7 and 15-21 of a 28-day treatment cycle.

In some embodiments, provided herein are methods for treating CMML in a subject with a therapeutically effective amount of an tipifarnib, based on the mutation status of KIR2DL1, KIR2DL3, KIR2DL4, KIR3DL1, and/or KIR3DL2 in a sample from the patient. In some embodiments, provided herein is a method of treating CMML in a subject including (a) determining a sample from the subject to have a mutant KIR2DL1, KIR2DL3, KIR2DL4, KIR3DL1, and/or KIR3DL2, and subsequently (b) administering tipifarnib to the subject at a dose in the range of 200 to 900 mg twice a day on days 1-21 of a 28-day treatment cycle.

In some embodiments, provided herein are methods for treating CMML in a subject with a therapeutically effective amount of an tipifarnib, based on the mutation status of KIR in a sample from the patient. In some embodiments, provided herein is a method of treating CMML in a subject including (a) determining a sample from the subject to have a mutant KIR, and subsequently (b) administering tipifarnib to the subject at a dose in the range of 200 to 900 mg twice a day on days 1-21 of a 28-day treatment cycle.

In some embodiments, provided herein are methods for treating CMML in a subject with a therapeutically effective amount of an tipifarnib, based on the mutation status of KIR2DL1, KIR2DL3, KIR2DL4, KIR3DL1, and/or KIR3DL2 in a sample from the patient. In some embodiments, provided herein is a method of treating CMML in a subject including (a) determining a sample from the subject to have a mutant KIR2DL1, KIR2DL3, KIR2DL4, KIR3DL1, and/or KIR3DL2, and subsequently (b) administering tipifarnib to the subject at a dose in the range of 200 to 900 mg twice a day on days 1-7 of a 28-day treatment cycle.

In some embodiments, provided herein are methods for treating CMML in a subject with a therapeutically effective amount of an tipifarnib, based on the mutation status of KIR in a sample from the patient. In some embodiments, provided herein is a method of treating CMML in a subject including (a) determining a sample from the subject to have a mutant KIR (e.g., a mutant KIR2DL1, KIR2DL3, KIR2DL4, KIR3DL1, and/or KIR3DL2), and subsequently (b) administering tipifarnib to the subject at a dose in the range of 200 to 900 mg twice a day on days 1-7 of a 28-day treatment cycle.

In some embodiments, the subject having a KIR-mutant cancer (e.g., a KIR-mutant CMML) who is selected for tipifarnib treatment receives a dose of 900 mg b.i.d. orally in alternate weeks (one week on, one week off) in repeated 4 week cycles.

In some embodiments, the subject having a KIR-mutant cancer (e.g., a KIR-mutant CMML) who is selected for tipifarnib treatment receives a dose of 600 mg b.i.d. orally in alternate weeks (one week on, one week off) in repeated 4 week cycles.

In some embodiments, the subject having a KIR-mutant cancer (e.g., a KIR-mutant CMML) who is selected for tipifarnib treatment receives a dose of 300 mg b.i.d. orally in alternate weeks (one week on, one week off) in repeated 4 week cycles.

In some embodiments, the methods further comprise administering a second therapy to the patient having a solid tumor with a mutation in a member of the KIR family (e.g., KIR2DL1, KIR2DL3, KIR2DL4, KIR3DL1, and/or KIR3DL2). In some embodiments, the second therapy is a chemotherapy, such as cisplatin, 5-FU, carboplatin, paclitaxel, or platinum-based doublet (e.g., cisplatin/5-FU or carboplatin/paclitaxel). In some embodiments, the second therapy is an anti-EGFR antibody therapy (e.g. Cetuximab, Panitumumab, Afatinib). In some embodiments, the second therapy is taxanes, methotrexate, and/or cetuximab. In some embodiments, the second therapy is a radiation therapy. In some embodiments, the second therapy include those targeting PI3K pathway: BKM120 (buparlisib)+cetuximab, BYL719+cetuximab, Temsirolimus+cetuximab, Rigosertib+cetuximab; those targeting MET pathway: Tivantinib+cetuximab, Ficlatuzumab+cetuximab; those targeting EGFR/HER3 pathway Afatinib+cetuximab±paclitaxel, Patritumab; those targeting FGFR pathway: BGJ398; those targeting CDK4/6-cell cycle pathway: Palbociclib, LEE011; RTK inhibitor: Anlotinib and chemotherapy: Oral Azacitidine. In some embodiments, the second therapy is an immunotherapy, such as anti-PD1 or anti-PDL1 antibodies. In some embodiments, the second therapy is a SRC family kinase inhibitor and/or a tyrosine kinase inhibitor (e.g, dasatinib). In some embodiments, the second therapy is dasatinib. In some embodiments, the second therapy is imatinib.

### 6. Examples

It is understood that modifications which do not substantially affect the activity of the various embodiments of this invention are also provided within the definition of the invention provided herein. Accordingly, the following examples are intended to illustrate but not limit the present invention. All of the references cited to herein are incorporated by reference in their entireties.

### Example I

### Tipifarnib Clinical Trial in PTCL.Patients

This example describes an ongoing Phase 2 clinical study of tipifarnib with the primary objective being to assess the antitumor activity in terms of Overall Response Rate (ORR) of tipifarnib in approximately 18-30 eligible subjects eligible subjects with Peripheral T-Cell Lymphoma (PTCL) (ClinicalTRials.gov identifier: NCT02464228).

Subjects receive tipifarnib administered at a starting dose of 300 mg, orally with food, twice a day (bid) on Days 1-21 in 28 day cycles (i.e. 3 weeks on/1 week off). Stepwise 100 mg dose reductions to control treatment-related, treatment-emergent toxicities were also allowed. Subjects who received tipifarnib bid on days 1-7 and days 15-21 in 28 day cycles during the conduct of earlier versions of this protocol were permitted to remain on that dose regimen at the discretion of the investigator. Alternatively, the subject was permitted to transition to receive a dose of 300 mg, orally with food, bid on days 1-21 of 28 day treatment cycles beginning on Day 1 of their next cycle. In the absence of unmanageable toxicities, subjects may continue to receive tipifarnib treatment for up to 12 months in the absence of disease progression and unmanageable toxicity. Treatment was permitted to continue beyond 12 months upon agreement of the Investigator and Sponsor.

Tumor assessments are performed at screening, at the Day 22 visit (±5 days) performed during Cycles 2, 4, 6 and once every approximately 12 weeks (cycles 9, 12, 15, etc.) thereafter, until disease progression.

Determination of objective tumor response is performed based on the Lugano Classification (Cheson 2014, Appendix II: The Lugano Classification) and/or measurable cutaneous disease according to the modified Severity Weighted Assessment Tool (mSWAT, Olsen 2011, Appendix III: Modified Severity Weighted Assessment Tool).

Upon disease progression, subjects are followed approximately every 12 weeks for survival until either death or 12 months after accrual of the last study subject, whichever occurs first. Information on subsequent anticancer therapy is also collected.

Primary outcome measures: objective response rate (ORR) following treatment with tipfarnib. [Time Frame: 2 years]. Response assessments according to IWC and/or mSWAT.

Secondary outcome measures: rate of progression free survival (PFS) [Time Frame: 2 years]; duration of response [Time Frame: 1 year]; number of patients that experience Adverse Events (AEs) [Time Frame: Until 30 days following end of study].

Detailed Description:

This Phase II study investigates the antitumor activity in terms of ORR of tipifarnib in approximately 18-30 eligible subjects with relapsed or refractory PTCL. The first 18 subjects were permitted to have the following PTCL sub-types: PTCL, not otherwise specified (PTCL-NOS), angioimmunoblastic T-cell lymphoma (AITL), ALK-positive and -negative anaplastic large cell lymphoma (ALCL), hepatosplenic T-cell lymphoma, enteropathy-associate T-cell lymphoma (EATL), extranodal natural killer (NK) T-cell lymphoma, nasal type and subcutaneous panniculitis-like T-cell lymphoma. The AITL expansion cohort (N=12) enrolls only subjects with AITL. Only consented subjects who meet all eligibility criteria were enrolled in the study. Eligible subjects received tipifarnib administered at a starting dose of 300 mg, orally with food, twice a day (bid) on Days 1-21 in 28 day cycles (i.e. 3 weeks on/1 week off). Stepwise 100 mg dose reductions to control treatment-related, treatment-emergent toxicities were also allowed. Subjects who received tipifarnib bid on days 1-7 and days 15-21 in 28 day cycles during the conduct of earlier versions of this protocol were permitted to remain on that dose regimen at the discretion of the investigator. Alternatively, the subject was permitted to transition to receive a dose of 300 mg, orally with food, bid on days 1-21 of 28 day treatment cycles beginning on Day 1 of their next cycle. In the absence of unmanageable toxicities, subjects may continue to receive tipifarnib treatment for up to 12 months in the absence of disease progression and unmanageable toxicity. Treatment was permitted to continue beyond 12 months upon agreement of the Investigator and Sponsor.

A two-stage study design was used for the first 18 subjects in order to minimize the number of study subjects treated if tipifarnib were considered not sufficiently efficacious to grant further development in this subject population. Tumor response assessments were conducted according to IWC and/or mSWAT criteria.

Tumor assessments were performed approximately every 8 weeks on cycles 2-6 and at least once approximately every 12 weeks thereafter (Cycles 9, 12, 15, etc.), and continued until disease progression. Upon disease progression, all subjects were followed approximately every 12 weeks for survival and the use of subsequent therapy until either death or 12 months after accrual of the last study subject, whichever occurred first. All subjects were followed-up for safety during treatment and up to approximately 30 days (30+/−7 days) after treatment discontinuation or until immediately before the initiation of another anti-cancer therapy, whichever occurred first.

Inclusion Criteria:

(1) Subject is at least 18 years of age.
(2) Diagnosis of PTCL according to the most recent edition of the World Health Organization (WHO) Classification of Tumors of Hematopoietic or Lymphoid Tissues, as follows: (a) Anaplastic large cell lymphoma (ALCL), ALK positive; (b) ALCL, ALK negative; (c) Angioimmunoblastic T-cell lymphoma (AITL); (d) Enteropathy-associated T-cell lymphoma; (e) Extranodal natural killer (NK) T-cell lymphoma, nasal type; (f) Hepatosplenic T-cell lymphoma; (g) Peripheral T-cell lymphoma, no otherwise specified (NOS); and (h) Subcutaneous panniculitis-like T-cell lymphoma. For enrollment into the AITL expansion cohort, subjects must have the diagnosis of AITL.
(3) Subject has relapsed or are refractory to at least 1 prior systemic cytotoxic therapy. Subjects must have received conventional therapy as a prior therapy.
(4) Subject has measurable disease according to the Lugano Classification and/or mSWAT.
(5) At least 2 weeks since the last systemic therapy regimen prior to enrollment. Subjects must have recovered to NCI CTCAE v. 4.03<Grade 2 from all acute toxicities (excluding Grade 2 toxicities that are not considered a safety risk by the Sponsor and Investigator) or toxicity must be deemed irreversible by the Investigator.
(6) At least 2 weeks since last radiotherapy if radiation was localized to the only site of measurable disease, unless there is documentation of disease progression of the irradiated site. Subjects must have recovered from all acute toxicities from radiotherapy.
(7) ECOG performance status of 0-2.
(8) Acceptable liver function: (a) Bilirubin ≤1.5 times upper limit of normal (x ULN); does not apply to subjects with Gilbert's syndrome diagnosed as per institutional guidelines, (b) AST (SGOT) and ALT (SGPT)≤3×ULN; if liver lymphoma present then ≤5×ULN is allowed.
(9) Acceptable renal function with serum creatinine ≤1.5×ULN or a calculated creatinine clearance ≥60 mL/min using the Cockcroft-Gault or Modification of Diet in Renal Disease formulas.
(10) Acceptable hematologic status: (a) ANC≥1000 cells/μL; (b) Platelet count ≥50,000/μL; (c) Hemoglobin ≥8.0 g/dL.
(11) Female subjects must be: Of non-child-bearing potential (surgically sterilized or at least 2 years post-menopausal); or If of child-bearing plf of child-bearing potential, subject must use an adequate method of contraception consisting of two-barrier method or one barrier method with a spermicide or intrauterine device. Both females and male subjects with female partners of child-bearing potential must agree to use an adequate method of contraception for 2 weeks prior to screening, during, and at least 4 weeks after last dose of study medication. Female subjects must have a negative serum or urine pregnancy test within 72 hours prior to start of study medication. Not breast feeding at any time during the study.
(12) Written and voluntary informed consent understood, signed and dated.

Exclusion Criteria:

(1) Diagnosis of any of the following: Precursor T-cell lymphoma or leukemia, AITL, T-cell prolymphocytic leukemia, T-cell large granular lymphocytic leukemia, Primary cutaneous type anaplastic large cell lymphoma, or Mycosis fungoide/Sezary syndrome.
(2) Ongoing treatment with an anticancer agent not contemplated in this protocol.
(3) Prior treatment (at least 1 full treatment cycle) with an FTase inhibitor.
(4) Any history of clinically relevant coronary artery disease or myocardial infarction within the last 3 years, New York Heart Association (NYHA) grade III or greater congestive heart failure, cerebro-vascular attack within the prior year, or current serious cardiac arrhythmia requiring medication except atrial fibrillation.
(5) Known central nervous system lymphoma.
(6) Stem cell transplant less than 3 months prior to enrollment.
(7) Non-tolerable ≥Grade 2 neuropathy or evidence of unstable neurological symptoms within 4 weeks of Cycle 1 Day 1. Non-tolerable grade 2 toxicities are defined as those with moderate symptoms that the subject is not able to endure for the conduct of instrumental activities of daily life or that persists ≥7 days.
(8) Major surgery, other than diagnostic surgery, within 2 weeks prior to Cycle 1 Day 1, without complete recovery.
(9) Other active malignancy requiring therapy such as radiation, chemotherapy, or immunotherapy.
(10) Active and uncontrolled bacterial, viral, or fungal infections, requiring systemic therapy. Known infection with human immunodeficiency virus (HIV), or an active infection with hepatitis B or hepatitis C.
(11) Subjects who have exhibited allergic reactions to tipifarnib, or structural compounds similar to tipifarnib or to its excipients. This includes hypersensitivity to imidazoles, such as clotrimazole, ketoconazole, miconazole and others in this drug class. Subjects with hypersensitivity to these agents will be excluded from enrollment.
(12) Concomitant disease or condition that could interfere with the conduct of the study, or that would, in the opinion of the investigator, pose an unacceptable risk to the subject in this study.
(13) The subject has legal incapacity or limited legal capacity.
(13) Dementia or significantly altered mental status that would limit the understanding or rendering of informed consent and compliance with the requirements of this protocol. Unwillingness or inability to comply with the study protocol for any reason.

Tumor assessments can be performed at screening, at the Day 22 visit (±5 days) performed during Cycles 2, 4, 6 and once every approximately 12 weeks (cycles 9, 12, 15, etc.) thereafter, until disease progression. Tumor assessments can be performed more frequently if deemed necessary by the investigator. A tumor assessment can be performed upon treatment discontinuation (End of Treatment visit) if the reason for discontinuation is other than disease progression and no tumor assessment was performed in the prior 8 weeks. Subjects who discontinued treatment for reasons other than disease progression were required to continue tumor assessments until disease progression, withdrawal of subject's consent or initiation of another anticancer therapy. Determination of objective tumor response is performed by the Investigator according to the Lugano Classification and/or mSWAT criteria.

### Example II

### Durable Responses in MR-Mutant PTCL Patients

In the Phase 2 clinical study of tipifarnib in patients with PTCL described in Example 1, KIR gene status was determined for 33 patients (PTCL-NOS (N=18) and AITL (N=15)). The KIR gene status of pretreatment biopsies from the 33 patients was determined using next generation whole exome sequencing, sometimes referred to as Next Generation Sequencing (“NGS”), and the single nucleotide variations (SNV) were analyzed according to the primary study endpoint of objective response. A high rate of inhibitory KIR mutation (SNV of expected maximal population frequency <1%) was observed in 16 AITL patients, and in particular, increased KIR-DL gene variation was observed in AITL patients who responded to tipifarnib treatment.

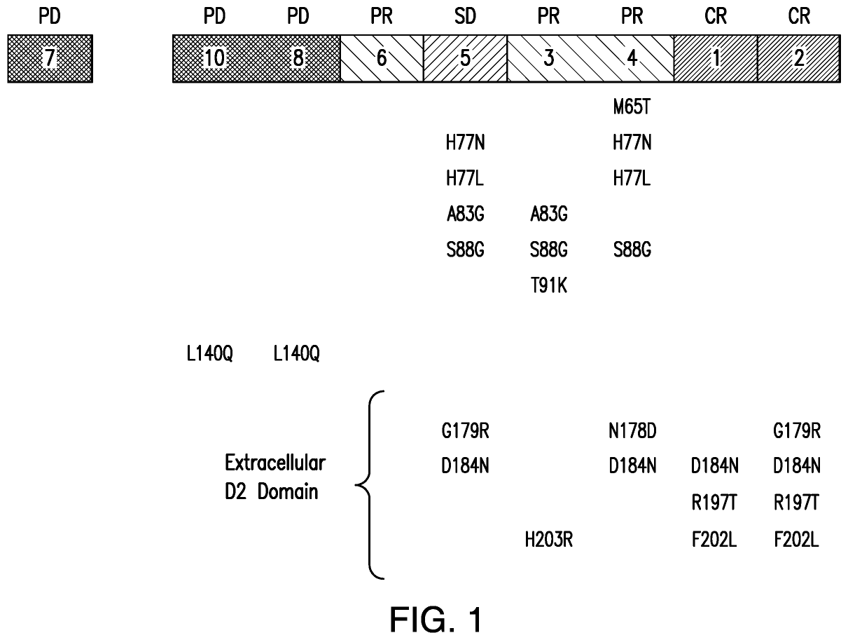

FIGS. 1-5 shows a graph for 9 patients of the total 33 patients and listing the mutations in KIR2DL1, KIR2DL3, KIR2DL4, KIR3DL1, and/or KIR3DL2, respectively, that were determined to be present in samples obtained from these patients (each of patients 1-8 and 10 having AITL), and the resulting response of said patients to treatment with tipifarnib. These data indicate that subjects with mutant KIR-DL genes, particularly in AITL patients, was associated with response to tipifarnib.

For example, FIG. 1 shows that the objective responses of these 9 patients carrying a D184N mutant of KIR2DL1 were 2 complete responses (CR), 1 partial response (PR), and 1 stabile disease (SD); that the objective responses of these 9 patients carrying a R197T mutant of KIR2DL1 were 2 complete responses (CR), that the objective responses of these 9 patients carrying a F202L mutant of KIR2DL1 were 2 complete responses (CR); that the objective responses of these 9 patients carrying a G179R mutant of KIR2DL1 were 1 complete response (CR) and 1 stabile disease response (SD); and that the objective responses of these 9 patients carrying a N178D mutant of KIR2DL1 was 1 partial response (PR).

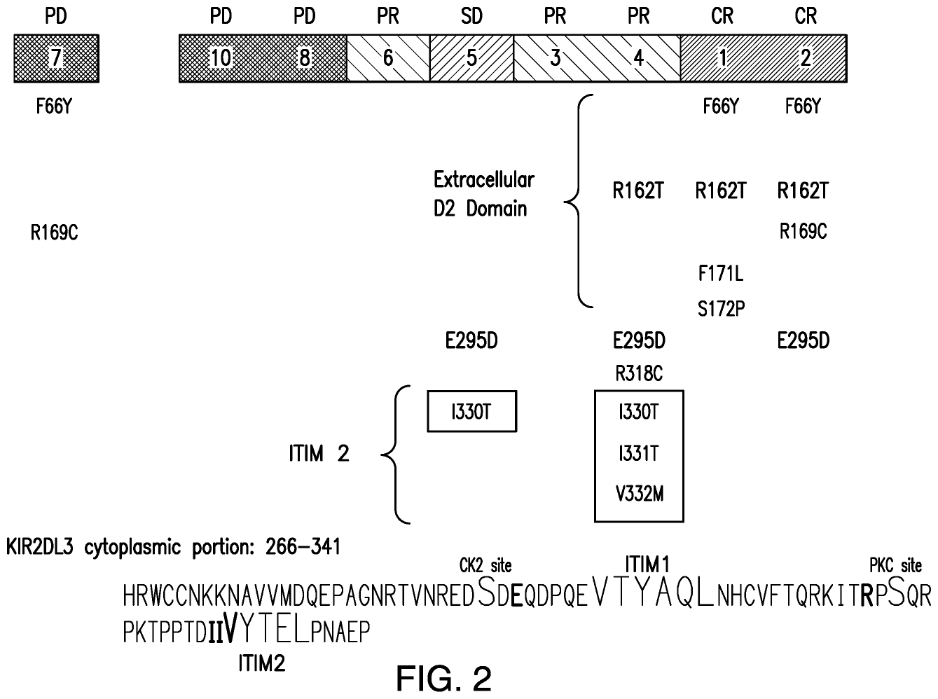

For example, FIG. 2 shows that the objective responses of these 9 patients carrying a R162T mutant of KIR2DL3 were 2 complete responses (CR) and 1 partial response (PR); that the objective responses of these 9 patients carrying a E295D mutant of KIR2DL3 were 1 complete response (CR), 1 partial response (PR), and 1 stabile disease response (SD).

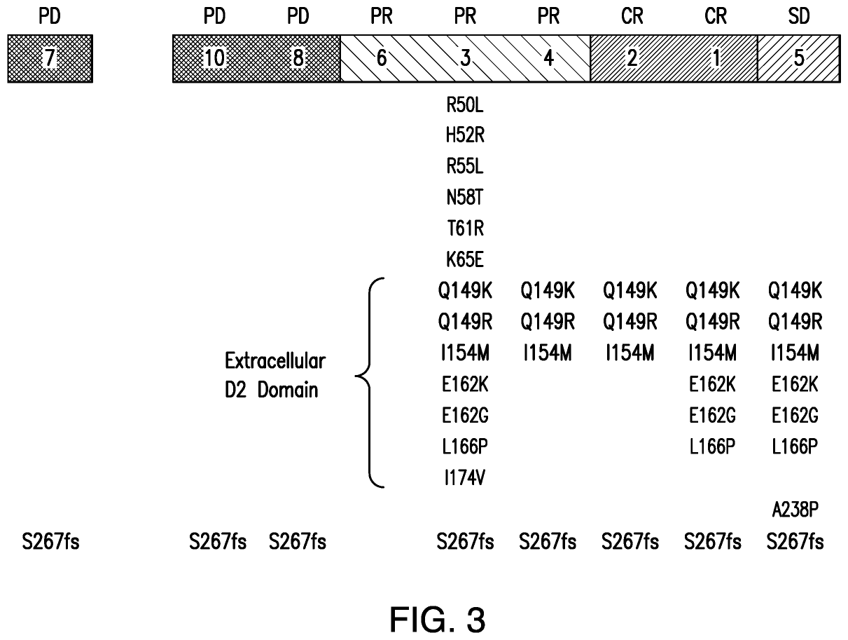

For example, FIG. 3 shows that the objective responses of these 9 patients carrying Q149K, Q149R, and I154M mutants of KIR2DL4 were 2 complete responses (CR), 2 partial responses (PR), and 1 stabile disease response (SD).

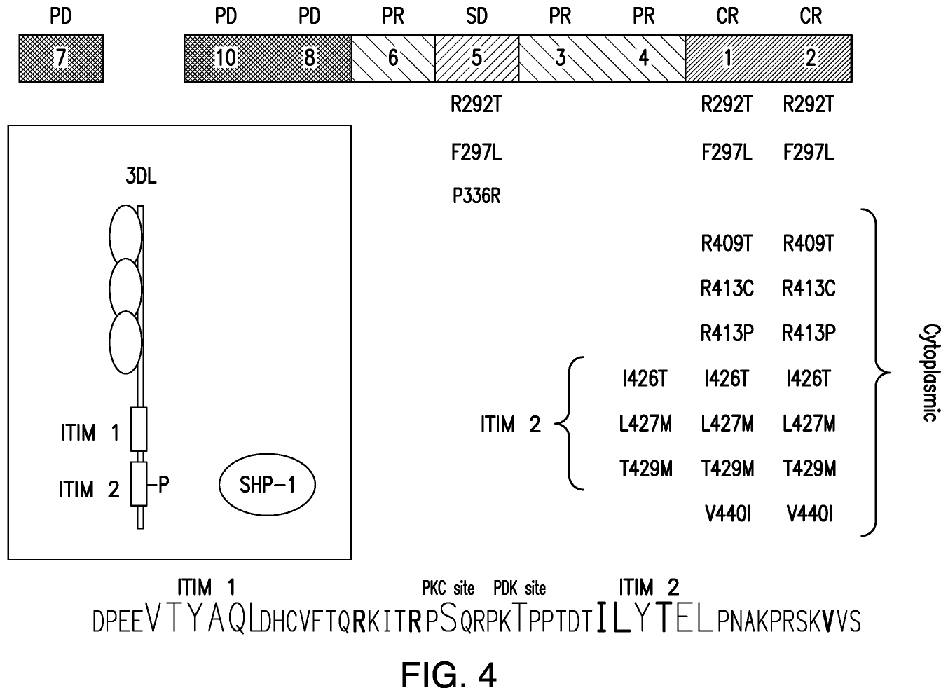

For example, FIG. 4 shows that notable mutations found in KIR3DL1, such as mutations in ITIM2 of KIR3DL1 (I426T, L427M, and T429M), potentially affecting SHP-1 binding, were found in patients 4, 1, and 2, who experienced PR, CR, and CR, respectively. The patients 1 and 2 having CR also had more extensive mutations in the cytoplasmic portion of KIR3DL1 in the vicinity of the PKC phosphorylation site.

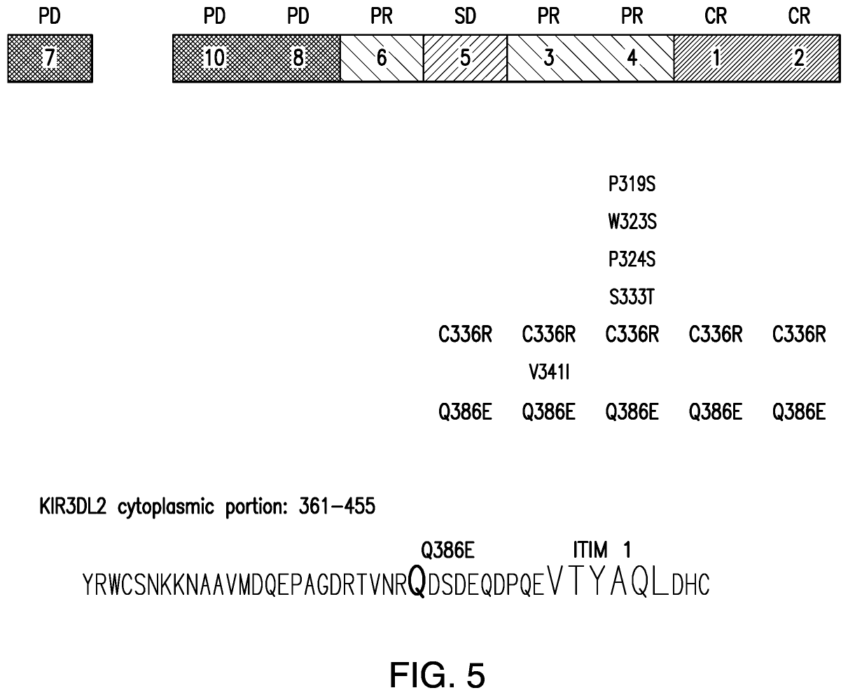

For example, FIG. 5 shows that the objective responses of these 9 patients carrying C336R and Q386E mutants of KIR3DL2 were 2 complete responses (CR), 2 partial responses (PR), and 1 stabile disease response (SD).

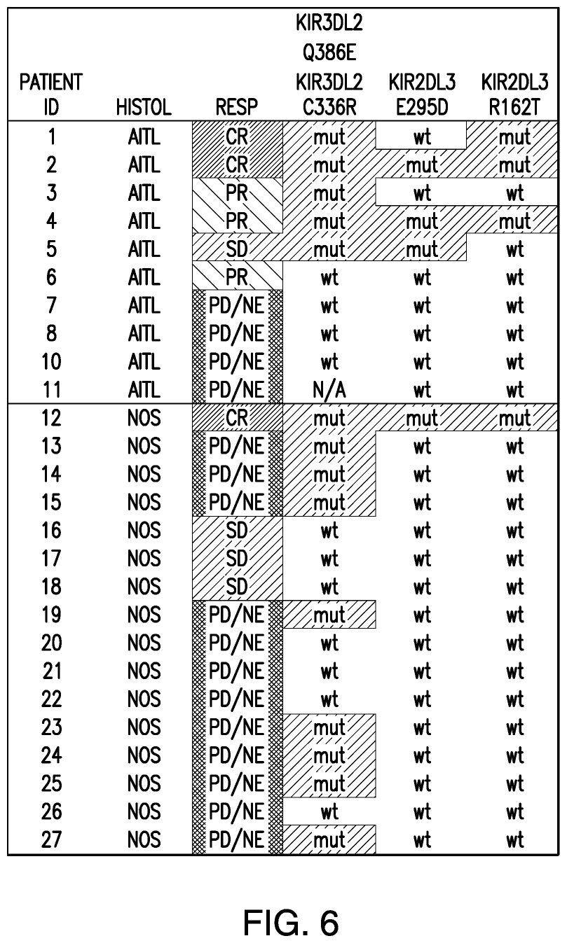

FIG. 6 shows that, of the first 26 patient samples evaluated of the total 33 patient samples, a total of 14 of 26 patient samples (54%) carried a Q386E mutant of KIR3DL2 (PTCL-NOS (N=9), AITL (N=5)). In addition, each of these 14 patients having the Q386E mutant of KIR3DL2 also had a C336R mutant of KIR3DL2. These 14 patients having the Q386E mutant of KIR3DL2 had the following objective responses: 3 CRs, 2 PRs, 1 SD, 8 PDs (36% ORR). When evaluated by disease type, the objective responses of these 14 patients carrying a Q386E mutant of KIR3DL2 were as follows: (a) 2 complete responses (CR), 2 partial responses (PR) and 1 stabile disease (SD) (80% ORR) were observed in AITL patients (N=5); and (b) 1 CR and 8 progressive disease (PD) (11% ORR) were observed in PTCL-NOS patients (N=9). Comparatively, patients (N=13) with wild type (wt) KIR3DL2 at position 386 had the following objective responses: 1 PR, 3 SD, and 9 PD (8% ORR).

From FIG. 4., it is noted that mutations were found in the ITIM2 of KIR3DL1 and KIR2DL3 (3 patients), and from FIG. 2 and FIG. 5 within the vicinity of or near the ITIM1 and CK2 phosphorylation sites of KIR2DL3 and KIR3DL2 (5 patients), respectively.

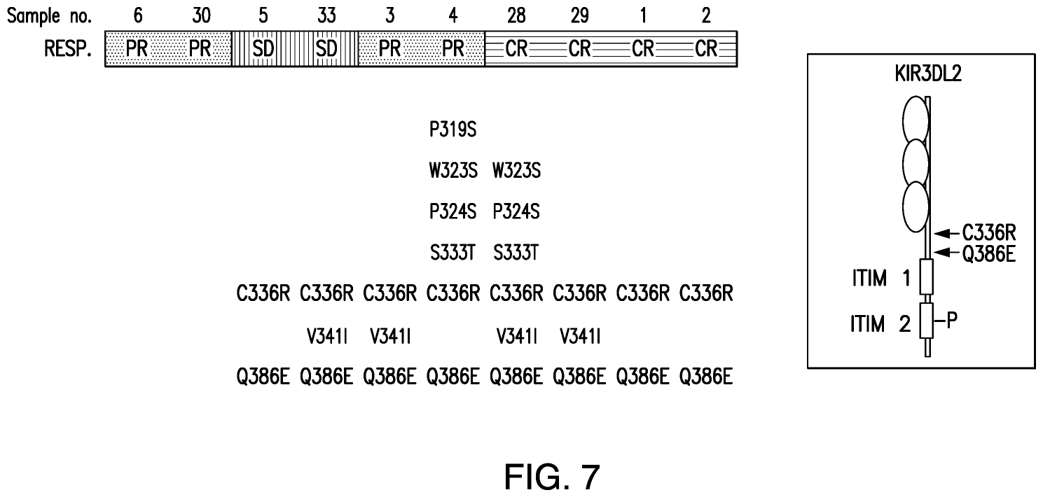

FIG. 7 shows a graph for a subset of 10 patients of the total 33 patients and listing the mutations in KIR3DL2 that were determined to be present in samples obtained from these patients (each patient of this subset having AITL), and the resulting response of said patients to treatment with tipifarnib. These data indicate that subjects with mutant KIR-DL genes, particularly KIR3DL2 in AITL patients, was associated with response to tipifarnib. For example, FIG. 7 shows that the objective responses of these 10 patients carrying C336R and Q386E mutants of KIR3DL2 were 4 complete responses (CR), 2 partial responses (PR), and 2 stabile disease responses (SD).

Upon analysis of the 15 pre-treatment AITL patient tumor samples, a significant association between whether the pre-treatment AITL tumor patient sample carried a Q386E mutant and a C336R mutant of KIR3DL2 (“KIR3DL2 C336R/Q386E mutant”) and clinical benefit from tipifarnib treatment was observed, with 8 of 15 patients that carried KIR3DL2 C336R/Q386E mutant responding to tipifarnib treatment as follows: 4 CRs, 2PRs, and 2 SDs (8/8 CR-PR-SD), relative to the remaining 7 of 15 patients that carried KIR3DL2 wild type responding to tipifarnib treatment as follows: 2PRs (2/7 CR-PR-SD) (p=0.009), which is presented in Table 2 below.

TABLE 2

| | KIR3DL2 | |
|---|---|---|
| | C336R/Q386E | KIR3DL2 |
| | mutant | wild type |
| N | 8 | 7 |
| Overall Best Response | | |
| Complete Response (CR) | 4 | — |
| Partial Response (PR) | 2 | 2 |
| Stable Disease (SD) | 2 | — |
| Progressive Disease (PD) | — | 5 |
| Not efficacy evaluable (NE) | — | — |
| Overall Response Rate (CR + PR) | 6/8 (75%) | 2/7 (29%) |
| 95% Cl | 35.9-95.4 | 4.6-64.1 |
| Clinical Benefit Rate (CR + PR + SD) | 8/8 (100%) | 2/7 (29%) |
| 95% Cl | 64.1-100.0 | 4.6-64.1 |

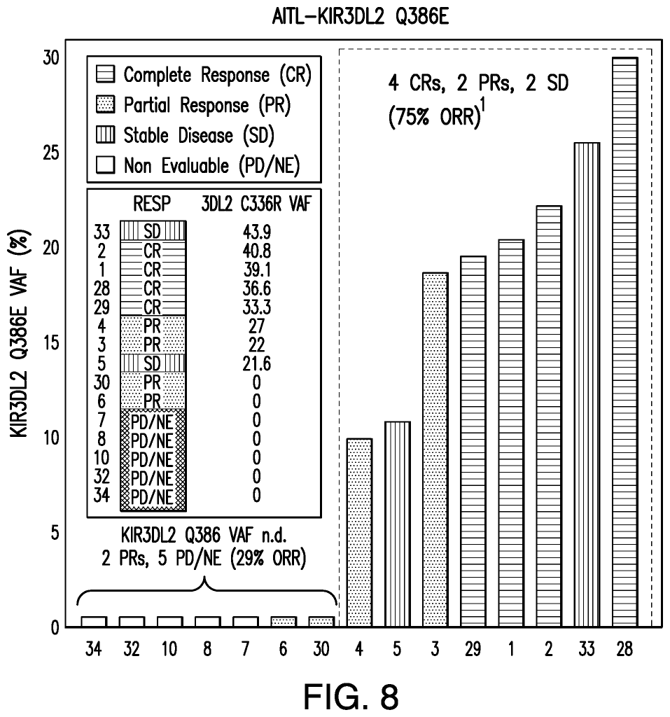

As determined by NGS, higher variant allele frequency (“VAF”) of the KIR3DL2 C336R/Q386E mutant was correlated with quality of the responses, and predictive of complete responses with tipifarnib treatment (Receiver Operator Curve AUC=0.94, p<0.0001, for KIR3DL2 Q386E VAF>19% and p<0.001 KIR3DL2 C336R VAF>27%). For example, as shown in FIG. 8, a KIR3DL2 C336R VAF of greater than 20%, or a KIR3DL2 Q386E VAF of greater than 5%, such as greater than 8%, or a combination of a KIR3DL2 C336R VAF of greater than 20% and a KIR3DL2 Q386E VAF of greater than 5%, such as greater than 8%, was predictive of a clinical benefit (response of CR, PR, or SD) upon tipifarnib treatment (ORR=Objective Response Rate by IWGC; KIR3DL2 mutant patients were 88% Caucasian, 75% male, 62% stage IV, 50% had B symptoms, and 88% had prior transplants; KIR3DL2 wt patients were 75% caucasian, 75% male, 75% stage IV, 50% had B symptoms, 37% had prior transplants; B symptoms: Fever, night sweats, and weight loss). From these data, the use of VAF of KIR-DL mutations/polymorphisms, for example VAF of KIR3DL2 C336R/Q3836E, may identify AITL patients who will be responsive to tipifarnib treatment.

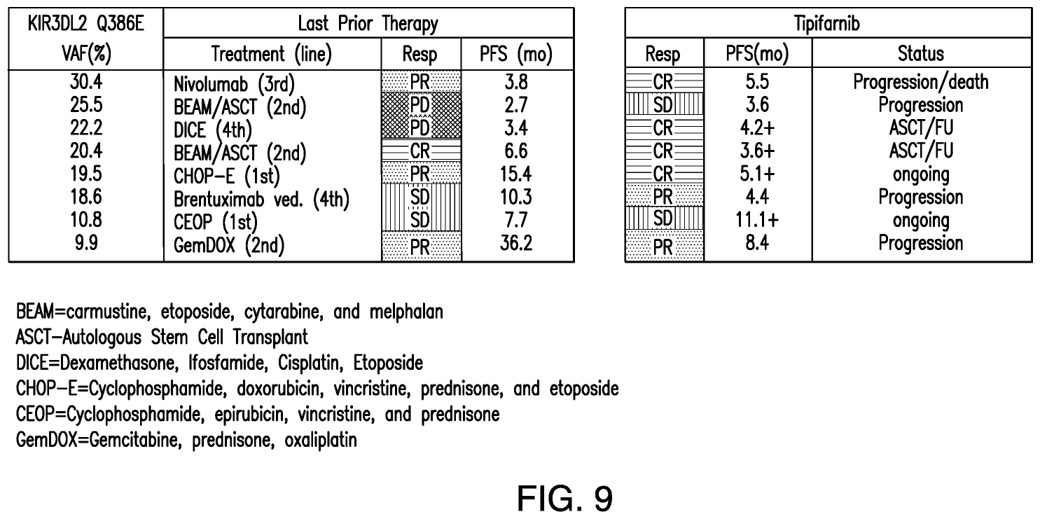

As shown in FIG. 9, upon analysis of the prior standard of care (SOC) treatment of the 8 AITL patients with KIR3DL2 gene variants, the presence of the KIR3DL2 variation (such as KIR3DL2 C336R, KIR3DL2 Q3836E, or KIR3DL2 C336R/Q3836E) in an AITL patient may be indicative of poor SOC treatment prognosis. Additionally, the presence of the KIR3DL2 variation in an AITL patient may be indicative of a better outcome upon tipifarnib treatment, relative to SOC treatment in their last prior line of therapy (e.g., Nivolumab, BEAM/ASCT, DICE, CHOP-E, Brentuximab ved., CEOP, or GemDOX).

Additional 11 patient samples from tipifarnib treated patients and the overall incidence of KIR-DL mutation in PTCL and other lymphomas are being investigated.

These data indicate that subjects with mutant KIR2DL and/or KIR3DL tumors appear to be more responsive to tipifarnib treatment than those with wild type KIR2DL and/or KIR3DL tumors. Additionally, the association of a particular KIR2DL and/or KIR3DL mutation with objective response may provide a robust method for the selection or stratification of PTCL, AITL, and other lymphoma patients who could benefit from tipifarnib therapy.

### INCORPORATION BY REFERENCE

Various references such as patents, patent applications, and publications are cited herein, the disclosures of which are hereby incorporated by reference herein in their entireties.

TABLE 1
Sequence Listing

| ID | SEQUENCE | Comments; Reference |
|---|---|---|
| SEQ ID | MSLLVVSMAC VGFFLLQGAW PHEGVHRKPS LLAHPGRLVK | An exemplary amino |
| NO: 1 | SEETVILQCW SDVMFEHFLL HREGMFNDTL RLIGEHHDGV | acid sequence: |
| | SKANFSISRM TQDLAGTYRC YGSVTHSPYQ VSAPSDPLDI | homo sapiens KIR2DL1, |
| | VIIGLYEKPS LSAQLGPTVL AGENVTLSCS SRSSYDMYHL | (GenBank: SPC71652.1) |
| | SREGEAHERR LPAGPKVNGT FQADFPLGPA THGGTYRCFG | |
| | SFHDSPYEWS KSSDPLLVSV TGNPSNSWPS PTEPSSKTGN | |
| | PRHLHILIGT SVVIILFILL FFLLHHWCSN KKNAAVMDQE | |
| | SAGNRTANSE DSDEQDPQEV TYTQLNHCVF TQRKITRPSQ | |
| | RPKTPPTDII VYTELPNAES RSKVVSCP | |
| SEQ ID | ATCCTGTGCG CTGCTGAGCT GAGCTCGGTC GCGGCTGCCT | Coding Sequence |
| NO: 2 | GTCTGCTCCG GCAGCACCAT GTCGCTCTTG GTCGTCAGCA | (CDS 1-1614) of homo |
| | TGGCGTGTGT TGGGTTCTTC TTGCTGCAGG GGGCCTGGCC | sapiens KIR2DL1, mRNA |
| | ACATGAGGGA GTCCACAGAA AACCTTCCCT CCTGGCCCAC | GenBank: NM_014218.3) |
| | CCAGGTCGCC TGGTGAAATC AGAAGAGACA GTCATCCTGC | corresponding encoding |
| | AGTGTTGGTC AGATGTCATG TTTGAACACT TCCTTCTGCA | sequence of |
| | CAGAGAGGGG ATGTTTAACG ACACTTTGCG CCTCATTGGA | SEQ ID NO. 1 |
| | GAACACCATG ATGGGGTCTC CAAGGCCAAC TTCTCCATCA | |
| | GTCGCATGAC GCAAGACCTG GCAGGGACCT ACAGATGCTA | |
| | CGGTTCTGTT ACTCACTCCC CCTATCAGGT GTCAGCTCCC | |
| | AGTGACCCTC TGGACATCGT GATCATAGGT CTATATGAGA | |
| | AACCTTCTCT CTCAGCCCAG CTGGGCCCCA CGGTTCTGGC | |
| | AGGAGAGAAT GTGACCTTGT CCTGCAGCTC CCGGAGCTCC | |
| | TATGACATGT ACCATCTATC CAGGGAAGGG GAGGCCCATG | |
| | AACGTAGGCT CCCTGCAGGG CCCAAGGTCA ACGGAACATT | |
| | CCAGGCTGAC TTTCCTCTGG GCCCTGCCAC CCACGGAGGG | |
| | ACCTACAGAT GCTTCGGCTC TTTCCATGAC TCTCCATACG | |
| | AGTGGTCAAA GTCAAGTGAC CCACTGCTTG TTTCTGTCAC | |
| | AGGAAACCCT TCAAATAGTT GGCCTTCACC CACTGAACCA | |
| | AGCTCCAAAA CCGGTAACCC CCGACACCTG CACATTCTGA | |
| | TTGGGACCTC AGTGGTCATC ATCCTCTTCA TCCTCCTCTT | |
| | CTTTCTCCTT CATCGCTGGT GCTCCAACAA AAAAAATGCT | |
| | GCGGTAATGG ACCAAGAGTC TGCAGGAAAC AGAACAGCGA | |
| | ATAGCGAGGA CTCTGATGAA CAAGACCCTC AGGAGGTGAC | |
| | ATACACACAG TTGAATCACT GCGTTTTCAC ACAGAGAAAA | |
| | ATCACTCGCC CTTCTCAGAG GCCCAAGACA CCCCCAACAG | |
| | ATATCATCGT GTACACGGAA CTTCCAAATG CTGAGTCCAG | |
| | ATCCAAAGTT GTCTCCTGCC CATGAGCACC ACAGTCAGGC | |
| | CTTGAGGGCG TCTTCTAGGG AGACAACAGC CCTGTCTCAA | |
| | AACCGGGTTG CCAGCTCCCA TGTACCAGCA GCTGGAATCT | |
| | GAAGGCGTGA GTCTGCATCT TAGGGCATCG ATCTTCCTCA | |
| | CACCACAAAT CTGAATGTGC CTCTCTCTTG CTTACAAATG | |
| | TCTAAGGTCC CCACTGCCTG CTGGAGAAAA AACACACTCC | |
| | TTTGCTTAAC CCACAGTTCT CCATTTCACT TGACCCCTGC | |
| | CCACCTCTCC AACCTAACTG GCTTACTTCC TAGTCTACTT | |
| | GAGGCTGCAA TCACACTGAG GAACTCACAA TTCCAAACAT | |
| | ACAAGAGGCT CCCTCTTAAC GCAGCACTTA GACACGTGTT | |
| | GTTCCACCTT CCCTCATGCT GTTCCACCTC CCCTCAGACT | |
| | AGCTTTCAGT CTTCTGTCAG CAGTAAAACT TATATATTTT | |
| | TTAAAATAAC TTCAATGTAG TTTTCCATCC TTCAAATAAA | |
| | CATGTCTGCC CCCA | |
| SEQ ID | MSLMVVSMVC VGFFLLQGAW PHEGVHRKPS LLAHPGPLVK | An exemplary amino |
| NO: 3 | SEETVILQCW SDVRFQHFLL HREGKFKDTL HLIGEHHDGV | acid sequence: |
| | SKANFSIGPM MQDLAGTYRC YGSVTHSPYQ LSAPSDPLDI | homo sapiens KIR2DL3, |
| | VITGLYEKPS LSAQPGPTVL AGESVTLSCS SRSSYDMYHL | (GenBank: NP_056952.2) |
| | SREGEAHERR FSAGPKVNGT FQADFPLGPA THGGTYRCFG | |
| | SFRDSPYEWS NSSDPLLVSV TGNPSNSWPS PTEPSSETGN | |
| | PRHLHVLIGT SVVIILFILL LFFLLHRWCC NKKNAVVMDQ | |
| | EPAGNRTVNR EDSDEQDPQE VTYAQLNHCV FTQRKITRPS | |
| | QRPKTPPTDI IVYTELPNAE P | |
| SEQ ID | AGCTGGGGCG CGGCCGCCTG TCTGCACAGA CAGCACCATG | Coding Sequence |
| NO: 4 | TCGCTCATGG TCGTCAGCAT GGTGTGTGTT GGGTTCTTCT | (CDS 1-1596) of homo |
| | TGCTGCAGGG GGCCTGGCCA CATGAGGGAG TCCACAGAAA | sapiens KIR2DL3, mRNA |
| | ACCTTCCCTC CTGGCCCACC CAGGTCCCCT GGTGAAATCA | GenBank: NM_015868.2) |
| | GAAGAGACAG TCATCCTGCA ATGTTGGTCA GATGTCAGGT | corresponding encoding |
| | TTCAGCACTT CCTTCTGCAC AGAGAAGGGA AGTTTAAGGA | sequence of |
| | CACTTTGCAC CTCATTGGAG AGCACCATGA TGGGGTCTCC | SEQ ID NO. 3 |
| | AAGGCCAACT TCTCCATCGG TCCCATGATG CAAGACCTTG | |
| | CAGGGACCTA CAGATGCTAC GGTTCTGTTA CTCACTCCCC | |
| | CTATCAGTTG TCAGCTCCCA GTGACCCTCT GGACATCGTC | |
| | ATCACAGGTC TATATGAGAA ACCTTCTCTC TCAGCCCAGC | |
| | CGGGCCCCAC GGTTCTGGCA GGAGAGAGCG TGACCTTGTC | |
| | CTGCAGCTCC CGGAGCTCCT ATGACATGTA CCATCTATCC | |
| | AGGGAGGGGG AGGCCCATGA ACGTAGGTTC TCTGCAGGGC | |
| | CCAAGGTCAA CGGAACATTC CAGGCCGACT TTCCTCTGGG | |
| | CCCTGCCACC CACGGAGGAA CCTACAGATG CTTCGGCTCT | |
| | TTCCGTGACT CTCCATACGA GTGGTCAAAC TCGAGTGACC | |
| | CACTGCTTGT TTCTGTCACA GGAAACCCTT CAAATAGTTG | |
| | GCCTTCACCC ACTGAACCAA GCTCCGAAAC CGGTAACCCC | |
| | AGACACCTGC ATGTTCTGAT TGGGACCTCA GTGGTCATCA | |
| | TCCTCTTCAT CCTCCTCCTC TTCTTTCTCC TTCATCGCTG | |
| | GTGCTGCAAC AAAAAAAATG CTGTTGTAAT GGACCAAGAG | |
| | CCTGCAGGGA ACAGAACAGT GAACAGGGAG GACTCTGATG | |
| | AACAAGACCC TCAGGAGGTG ACATATGCAC AGTTGAATCA | |
| | CTGCGTTTTC ACACAGAGAA AAATCACTCG CCCTTCTCAG | |
| | AGGCCCAAGA CACCCCCAAC AGATATCATC GTGTACACGG | |
| | AACTTCCAAA TGCTGAGCCC TGATCCAAAG TTGTCTCCTG | |
| | CCCATGAGCA CCACAGTCAG GCCTTGAGGG GATCTTCTAG | |
| | GGAGACAACA GCCCTGTCTC AAAACTGGGT TGCCAGCTCC | |
| | AATGTACCAG CAGCTGGAAT CTGAAGGCGT GAGTCTGCAT | |
| | CTTAGGGCAT CGCTCTTCCT CACACCACAA ATCTGAACGT | |
| | GCCTCTCCCT TGCTTACAAA TGTCTAAGGT CCCCACTGCC | |
| | TGCTGGAGAG AAAACACACT CCTTTGCTTA GCCCACAATT | |
| | CTCCATTTCA CTTGACCCCT GCCCACCTCT CCAACCTAAC | |
| | TGGCTTACTT CCTAGTCTAC TTGAGGCTGC AATCACACTG | |
| | AGGAACTCAC AATTCCAAAC ATACAAGAGG CTCCCTCTTA | |
| | ACACGGCACT TAGACACGTG CTGTTCCACC TTCCCTCATG | |
| | CTGTTCCACC TCCCCTCAGA CTAGCTTTCA GCCTTCTGTC | |
| | AGCAGTAAAA CTTATATATT TTTTAAAATA ATTTCAATGT | |
| | AGTTTTCCCT CCTTCAAATA AACATGTCTG CCCTCA | |
| SEQ ID | MSMSPTVIIL ACLGFFLDQS VWAHVGGQDK PFCSAWPSAV | An exemplary amino |
| NO: 5 | VPQGGHVTLR CHYRRGFNIF TLYKKDGVpV pELYNRIFWN | acid sequence: |
| | SFLISPVTPA HAGTYRCRGF HPHSPTEWSA PSNPLVIMVT | homo sapiens KIR2DL4, |
| | GLYEKPSLTA RPGPTVRAGE NVTLSCSSQS SFDIYHLSRE | (GenBank: NP_002246.5) |
| | GEAHELRLPA VPSINGTFQA DFPLGPATHG ETYRCFGSFH | |
| | GSPYEWSDPS DPLPVSVTGN PSSSWPSPTE PSFKTGIARH | |
| | LHAVIRYSVA IILFTILPFF LLHRWCSKKK NAAVMNQEPA | |
| | GHRTVNREDS DEQDPQEVTY AQLDHCIFTQ RKITGPSQRS | |
| | KRPSTDTSVC IELPNAEPRA LSPAHEHHSQ ALMGSSRETT | |
| | ALSQTQLASS NVPAAGI | |
| SEQ ID | AGTCGAGCCG AGTCACTGCG TCCTGGCAGC AGAAGCTGCA | Coding Sequence |
| NO: 6 | CCATGTCCAT GTCACCCACG GTCATCATCC TGGCATGTCT | (CDS 1-1582) of homo |
| | TGGGTTCTTC TTGGACCAGA GTGTGTGGGC ACACGTGGGT | sapiens KIR2DL4, mRNA |
| | GGTCAGGACA AGCCCTTCTG CTCTGCCTGG CCCAGCGCTG | GenBank: NM_002255.6) |
| | TGGTGCCTCA AGGAGGACAC GTGACTCTTC GGTGTCACTA | corresponding encoding |
| | TCGTCGTGGG TTTAACATCT TCACGCTGTA CAAGAAAGAT | sequence of |
| | GGGGTCCCTG TCCCTGAGCT CTACAACAGA ATATTCTGGA | SEQ ID NO. 5 |
| | ACAGTTTCCT CATTAGCCCT GTGACCCCAG CACACGCAGG | |
| | GACCTACAGA TGTCGAGGTT TTCACCCGCA CTCCCCCACT | |
| | GAGTGGTCGG CACCCAGCAA CCCCCTGGTG ATCATGGTCA | |
| | CAGGTCTATA TGAGAAACCT TCGCTTACAG CCCGGCCGGG | |
| | CCCCACGGTT CGCGCAGGAG AGAACGTGAC CTTGTCCTGC | |
| | AGCTCCCAGA GCTCCTTTGA CATCTACCAT CTATCCAGGG | |
| | AGGGGGAAGC CCATGAACTT AGGCTCCCTG CAGTGCCCAG | |
| | CATCAATGGA ACATTCCAGG CCGACTTCCC TCTGGGTCCT | |
| | GCCACCCACG GAGAGACCTA CAGATGCTTC GGCTCTTTCC | |
| | ATGGATCTCC CTACGAGTGG TCAGACCCGA GTGACCCACT | |
| | GCCTGTTTCT GTCACAGGAA ACCCTTCTAG TAGTTGGCCT | |
| | TCACCCACTG AACCAAGCTT CAAAACTGGT ATCGCCAGAC | |
| | ACCTGCATGC TGTGATTAGG TACTCAGTGG CCATCATCCT | |
| | CTTTACCATC CTTCCCTTCT TTCTCCTTCA TCGCTGGTGC | |
| | TCCAAAAAAA AAAATGCTGC TGTAATGAAC CAAGAGCCTG | |
| | CGGGACACAG AACAGTGAAC AGGGAGGACT CTGATGAACA | |
| | AGACCCTCAG GAGGTGACAT ACGCACAGTT GGATCACTGC | |
| | ATTTTCACAC AGAGAAAAAT CACTGGCCCT TCTCAGAGGA | |
| | GCAAGAGACC CTCAACAGAT ACCAGCGTGT GTATAGAACT | |
| | TCCAAATGCT GAGCCCAGAG CGTTGTCTCC TGCCCATGAG | |
| | CACCACAGTC AGGCCTTGAT GGGATCTTCT AGGGAGACAA | |
| | CAGCCCTGTC TCAAACCCAG CTTGCCAGCT CTAATGTACC | |
| | AGCAGCTGGA ATCTGAAGGC GTGAGTCTCC ATCTTAGAGC | |
| | ATCACTCTTC CTCACACCAC AAATCTGGTG CCTGTCTCTT | |
| | GCTTACCAAT GTCTAAGGTC CCCACTGCCT GCTGCAGAGA | |
| | AAACACACTC CTTTGCTTAG CCCACAATTC TCTATTTCAC | |
| | TTGACCCCTG CCCACCTCTC CAACCTAACT GGCTTACTTC | |
| | CTAGTCTACT TGAGGCTGCA ATCACACTGA GGAACTCACA | |
| | ATTCCAAACA TACAAGAGGC TCTCTCTTAA CACGGCACTT | |
| | AGACACGTGC TGTTCCACCT TCCCTCGTGC TGTTCCACCT | |
| | TTCCTCAGAC TATTTTTCAG CCTTCTGGCA TCAGCAAACC | |
| | TTATAAAATT TTTTTGATTT CAGTGTAGTT CTCTCCTCTT | |
| | CAAATAAACA TGTCTGCCTT CA | |
| SEQ ID | MSLMVVSMAC VGLFLVQRAG PHMGGQDKPF LSAWPSAVVP | An exemplary amino |
| NO: 7 | RGGHVTLRCH YRHRFNNFML YKEDRIHIPI FHGRIFQESF | acid sequence: |
| | NMSPVTTAHA GNYTCRGSHP HSPTGWSAPS NPVVIMVTGN | homo sapiens KIR3DL1, |
| | HRKPSLLAHP GPLVKSGERV ILQCWSPIMF EHFFLHKEGI | (GenBank: NP_037421.2) |
| | SKDPSRLVGQ IHDGVSKANF SIGPMMLALA GTYRCYGSVT | |
| | HTPYQLSAPS DPLDIVVTGP YEKPSLSAQP GPKVQAGESV | |
| | TLSCSSRSSY DMYHLSREGG AHERRLPAVR KVNRTFQADF | |
| | PLGPATHGGT YRCFGSFRHS PYEWSDPSDP LLVSVTGNPS | |
| | SSWPSPTEPS SKSGNPRHLH ILIGTSVVII LFILLLFFLL | |
| | HLWCSNKKNA AVMDQEPAGN RTANSEDSDE QDPEEVTYAQ | |
| | LDHCVFTQRK ITRPSQRPKT PPTDTILYTE LPNAKPRSKV | |
| | VSCP | |
| SEQ ID | ATAACATCCT GTGCGCTGCT GAGCTGAGCT GGGGCGCAGC | Coding Sequence |
| NO: 8 | CGCCTGTCTG CACCGGCAGC ACCATGTCGC TCATGGTCGT | (CDS 1-1986) of homo |
| | CAGCATGGCG TGTGTTGGGT TGTTCTTGGT CCAGAGGGCC | sapiens KIR3DL1, mRNA |
| | GGTCCACACA TGGGTGGTCA GGACAAACCC TTCCTGTCTG | GenBank: NM_013289.2) |
| | CCTGGCCCAG CGCTGTGGTG CCTCGAGGAG GACACGTGAC | corresponding encoding |
| | TCTTCGGTGT CACTATCGTC ATAGGTTTAA CAATTTCATG | sequence of |
| | CTATACAAAG AAGACAGAAT CCACATTCCC ATCTTCCATG | SEQ ID NO. 7 |
| | GCAGAATATT CCAGGAGAGC TTCAACATGA GCCCTGTGAC | |
| | CACAGCACAT GCAGGGAACT ACACATGTCG GGGTTCACAC | |
| | CCACACTCCC CCACTGGGTG GTCGGCACCC AGCAACCCCG | |
| | TGGTGATCAT GGTCACAGGA AACCACAGAA AACCTTCCCT | |
| | CCTGGCCCAC CCAGGTCCCC TGGTGAAATC AGGAGAGAGA | |
| | GTCATCCTGC AATGTTGGTC AGATATCATG TTTGAGCACT | |
| | TCTTTCTGCA CAAAGAGGGG ATCTCTAAGG ACCCCTCACG | |
| | CCTCGTTGGA CAGATCCATG ATGGGGTCTC CAAGGCCAAT | |
| | TTCTCCATCG GTCCCATGAT GCTTGCCCTT GCAGGGACCT | |
| | ACAGATGCTA CGGTTCTGTT ACTCACACCC CCTATCAGTT | |
| | GTCAGCTCCC AGTGATCCCC TGGACATCGT GGTCACAGGT | |
| | CCATATGAGA AACCTTCTCT CTCAGCCCAG CCGGGCCCCA | |
| | AGGTTCAGGC AGGAGAGAGC GTGACCTTGT CCTGTAGCTC | |
| | CCGGAGCTCC TATGACATGT ACCATCTATC CAGGGAGGGG | |
| | GGAGCCCATG AACGTAGGCT CCCTGCAGTG CGCAAGGTCA | |
| | ACAGAACATT CCAGGCAGAT TTCCCTCTGG GCCCTGCCAC | |
| | CCACGGAGGG ACCTACAGAT GCTTCGGCTC TTTCCGTCAC | |
| | TCTCCCTACG AGTGGTCAGA CCCGAGTGAC CCACTGCTTG | |
| | TTTCTGTCAC AGGAAACCCT TCAAGTAGTT GGCCTTCACC | |
| | CACAGAACCA AGCTCCAAAT CTGGTAACCC CAGACACCTG | |
| | CACATTCTGA TTGGGACCTC AGTGGTCATC ATCCTCTTCA | |
| | TCCTCCTCCT CTTCTTTCTC CTTCATCTCT GGTGCTCCAA | |
| | CAAAAAAAAT GCTGCTGTAA TGGACCAAGA GCCTGCAGGG | |
| | AACAGAACAG CCAACAGCGA GGACTCTGAT GAACAAGACC | |
| | CTGAGGAGGT GACATACGCA CAGTTGGATC ACTGCGTTTT | |
| | CACACAGAGA AAAATCACTC GCCCTTCTCA GAGGCCCAAG | |
| | ACACCCCCTA CAGATACCAT CTTGTACACG GAACTTCCAA | |
| | ATGCTAAGCC CAGATCCAAA GTTGTCTCCT GCCCATGAGC | |
| | ACCACAGTCA GGCCTTGAGG ACGTCTTCTA GGGAGACAAC | |
| | AGCCCTGTCT CAAAACCGAG TTGCCAGCTC CCATGTACCA | |
| | GCAGCTGGAA TCTGAAGGCG TGAGTCTTCA TCTTAGGGCA | |
| | TCGCTCCTCC TCACGCCACA AATCTGGTGC CTCTCTCTTG | |
| | CTTACAAATG TCTAGGTCCC CACTGCCTGC TGGAAAGAAA | |
| | ACACACTCCT TTGCTTAGCC CACAGTTCTC CATTTCACTT | |
| | GACCCCTGCC CACCTCTCCA ACCTAACTGG CTTACTTCCT | |
| | AGTCTACTTG AGGCTGCAAT CACACTGAGG AACTCACAAT | |
| | TCCAAACATA CAAGAGGCTC CCTCTTGACG TGGCACTTAC | |
| | CCACGTGCTG TTCCACCTTC CCTCATGCTG TTTCACCTTT | |
| | CTTCGGACTA TTTTCCAGCC TTCTGTCAGC AGTGAAACTT | |
| | ATAAAATTTT TTGTGATTTC AATGTAGCTG TCTCCTCTTC | |
| | AAATAAACAT GTCTGCCCTC AAAAAAAAAA AAAAAAAAAA | |
| | AAAAAAAAAA AAAAAAAAAA AAAAAAAAAA AAAAAAAAAA | |
| | AAAAAAAAAA AAAAAAAAAA AAAAAA | |
| SEQ ID | MSLTVVSMAC VGFFLLQGAW PLMGGQDKPF LSARPSTVVP | An exemplary amino |
| NO: 9 | RGGHVALQCH YRRGFNNFML YKEDRSHVPI FHGRIFQESF | acid sequence: |
| | IMGPVTPAHA GTYRCRGSRP HSLTGWSAPS NPLVIMVTGN | homo sapiens KIR3DL2, |
| | HRKPSLLAHP GPLLKSGETV ILQCWSDVMF EHFFLHREGI | (GenBank: NP_006728.2) |
| | SEDPSRLVGQ IHDGVSKANF SIGPLMPVLA GTYRCYGSVP | |
| | HSPYQLSAPS DPLDIVITGL YEKPSLSAQP GPTVQAGENV | |
| | TLSCSSWSSY DIYHLSREGE AHERRLRAVP KVNRTFQADF | |
| | PLGPATHGGT YRCFGSFRAL PCVWSNSSDP LLVSVTGNPS | |
| | SSWPSPTEPS SKSGICRHLH VLIGTSVVIF LFILLLFFLL | |
| | YRWCSNKKNA AVMDQEPAGD RTVNRQDSDE QDPQEVTYAQ | |
| | LDHCVFIQRK ISRPSQRPKT PLTDTSVYTE LPNAEPRSKV | |
| | VSCPRAPQSG LEGVF | |
| SEQ ID | GGGGCGCGGC CTCCTGTCTG CACCGGCAGC ACCATGTCGC | Coding Sequence |
| NO: 10 | TCACGGTCGT CAGCATGGCG TGCGTTGGGT TCTTCTTGCT | (CDS 1-1885) of homo |
| | GCAGGGGGCC TGGCCACTCA TGGGTGGTCA GGACAAACCC | sapiens KIR3DL2, mRNA |
| | TTCCTGTCTG CCCGGCCCAG CACTGTGGTG CCTCGAGGAG | GenBank: NM_006737.3) |
| | GACACGTGGC TCTTCAGTGT CACTATCGTC GTGGGTTTAA | corresponding encoding |
| | CAATTTCATG CTGTACAAAG AAGACAGAAG CCACGTTCCC | sequence of |
| | ATCTTCCACG GCAGAATATT CCAGGAGAGC TTCATCATGG | SEQ ID NO. 9 |
| | GCCCTGTGAC CCCAGCACAT GCAGGGACCT ACAGATGTCG | |
| | GGGTTCACGC CCACACTCCC TCACTGGGTG GTCGGCACCC | |
| | AGCAACCCCC TGGTGATCAT GGTCACAGGA AACCACAGAA | |
| | AACCTTCCCT CCTGGCCCAC CCAGGGCCCC TGCTGAAATC | |
| | AGGAGAGACA GTCATCCTGC AATGTTGGTC AGATGTCATG | |
| | TTTGAGCACT TCTTTCTGCA CAGAGAGGGG ATCTCTGAGG | |
| | ACCCCTCACG CCTCGTTGGA CAGATCCATG ATGGGGTCTC | |
| | CAAGGCCAAC TTCTCCATCG GTCCCTTGAT GCCTGTCCTT | |
| | GCAGGAACCT ACAGATGTTA TGGTTCTGTT CCTCACTCCC | |
| | CCTATCAGTT GTCAGCTCCC AGTGACCCCC TGGACATCGT | |
| | GATCACAGGT CTATATGAGA AACCTTCTCT CTCAGCCCAG | |
| | CCGGGCCCCA CGGTTCAGGC AGGAGAGAAC GTGACCTTGT | |
| | CCTGTAGCTC CTGGAGCTCC TATGACATCT ACCATCTGTC | |
| | CAGGGAAGGG GAGGCCCATG AACGTAGGCT CCGTGCAGTG | |
| | CCCAAGGTCA ACAGAACATT CCAGGCAGAC TTTCCTCTGG | |
| | GCCCTGCCAC CCACGGAGGG ACCTACAGAT GCTTCGGCTC | |
| | TTTCCGTGCC CTGCCCTGCG TGTGGTCAAA CTCAAGTGAC | |
| | CCACTGCTTG TTTCTGTCAC AGGAAACCCT TCAAGTAGTT | |
| | GGCCTTCACC CACAGAACCA AGCTCCAAAT CTGGTATCTG | |
| | CAGACACCTG CATGTTCTGA TTGGGACCTC AGTGGTCATC | |
| | TTCCTCTTCA TCCTCCTCCT CTTCTTTCTC CTTTATCGCT | |
| | GGTGCTCCAA CAAAAAGAAT GCTGCTGTAA TGGACCAAGA | |
| | GCCTGCGGGG GACAGAACAG TGAATAGGCA GGACTCTGAT | |
| | GAACAAGACC CTCAGGAGGT GACGTACGCA CAGTTGGATC | |
| | ACTGCGTTTT CATACAGAGA AAAATCAGTC GCCCTTCTCA | |
| | GAGGCCCAAG ACACCCCTAA CAGATACCAG CGTGTACACG | |
| | GAACTTCCAA ATGCTGAGCC CAGATCCAAA GTTGTCTCCT | |
| | GCCCACGAGC ACCACAGTCA GGTCTTGAGG GGGTTTTCTA | |
| | GGGAGACAAC AGCCCTGTCT CAAAACCAGG TTGCCAGATC | |
| | CAATGAACCA GCAGCTGGAA TCTGAAGGCA TCAGTCTGCA | |
| | TCTTAGGGGA TCGCTCTTCC TCACACCACG AATCTGAACA | |
| | TGCCTCTCTC TTGCTTACAA ATGCCTAAGG TCGCCACTGC | |
| | CTGCTGCAGA GAAAACACAC TCCTTTGCTT AGCCCACAAG | |
| | TATCTATTTC ACTTGACCCC TGCCCACCTC TCCAACCTAA | |
| | CTGGCTTACT TCCTAGTCCT ACTTGAGGCT GCAATCACAC | |
| | TGAGGAACTC ACAATTCCAA ACATACAAGA GGCTCCCTCT | |
| | TAACACGGCA CTTACACACT TGCTGTTCCA CCTTCCCTCA | |
| | TGCTGTTCCA CCTCCCCTCA GACTATCTTT CAGCCTTCTG | |
| | TCATCAGTAA AATTTATAAA TTTTTTTTAT AACTTCAGTG | |
| | TAGCTCTCTC CTCTTCAAAT AAACATGTCT GCCCTCATGG | |
| | TTTCG | |

We claim:

1. A method of treating a cancer in a subject in need thereof, said method comprising administering a therapeutically effective amount of a farnesyltransferase inhibitor (FTI) to said subject, wherein the cancer is a cancer known to have or determined to have a mutation in a member of the KIR family selected from the group consisting of: KIR2DL1, KIR2DL3, KIR2DL4, KIR3DL1, and KIR3DL2.

2. The method of claim 1, wherein the KIR2DL1, KIR2DL3, KIR2DL4, KIR3DL1, and/or KIR3DL2, has two of more mutations comprising two or more modifications at two or more codons encoding two or more amino acids in the extracellular domain, at two or more codons encoding two or more amino acids in the cytoplasmic domain, or combinations thereof.

3. The method of any one of claims 1-2, wherein the KIR2DL1, KIR2DL3, KIR2DL4, KIR3DL1, and/or KIR3DL2, has three of more mutations comprising three or more modifications at three or more codons encoding three or more amino acids in the extracellular domain, at three or more codons encoding three or more amino acids in the cytoplasmic domain, or combinations thereof.

4. The method of any one of claims 1-3, wherein the FTI, optionally tipifarnib, is selectively administered to a subject to treat the KIR-mutant cancer, and wherein the KIR-mutant cancer has or comprises a mutation in KIR2DL1.

5. The method of any one of claims 1-4, wherein the mutation is or comprises a modification in a codon of KIR2DL1 encoding an amino acid in the extracellular domain.

6. The method of claim 5, wherein the mutation is or comprises a modification in a codon of KIR2DL1 encoding an amino acid in the extracellular domain selected from a group consisting of: M65, H77, A83, S88, T91, L140, N178, G179, D184, R197, F202, and H203.

7. The method of any one of claims 5-6, wherein the mutation in the extracellular domain of KIR2DL1 is selected from a group consisting of: M65T, H77N, H77L, A83G, S88G, T91K, L140Q, N178D, G179R, D184N, R197T, F202L, and H203R.

8. The method of any one of claims 5-7, wherein the mutation is or comprises modifications in two or more, or three or more, codons of KIR2DL1 encoding two or more, or three or more, amino acids in the extracellular domain selected from a group consisting of: M65, H77, A83, S88, T91, L140, N178, G179, D184, R197, F202, and H203.

9. The method of any one of claims 5-8, wherein the extracellular domain of KIR2DL1 has two or more, or three or more, mutations selected from a group consisting of: M65T, H77N, H77L, A83G, S88G, T91K, L140Q, N178D, G179R, D184N, R197T, F202L, and H203R.

10. The method of any one of claims 1-9, wherein the mutation is or comprises a modification in a codon of KIR2DL1 encoding an amino acid in the extracellular D2 domain.

11. The method of claim 10, wherein the mutation is or comprises a modification in a codon of KIR2DL1 encoding an amino acid in the extracellular D2 domain selected from a group consisting of: N178, G179, D184, R197, F202, and H203.

12. The method of any one of claims 10-11, wherein the mutation in the extracellular D2 domain of KIR2DL1 is selected from a group consisting of: N178D, G179R, D184N, R197T, F202L, and H203R.

13. The method of any one of claims 10-11, wherein the mutation is or comprises modifications in two or more, or three or more, codons of KIR2DL1 encoding two or more, or three or more, amino acids in the extracellular D2 domain selected from a group consisting of: N178, G179, D184, R197, and F202.

14. The method of any one of claims 10-13, wherein the extracellular D2 domain of KIR2DL1 has two or more, or three or more, mutations selected from a group consisting of: N178D, G179R, D184N, R197T, and F202L.

15. The method of any one of claims 10-14, wherein the mutation is or comprises a modification in a codon of KIR2DL1 encoding amino acid N178 in the extracellular D2 domain.

16. The method of any one of claims 10-15, wherein the mutation in the extracellular D2 domain of KIR2DL1 is or comprises the N178D.

17. The method of any one of claims 10-16, wherein the mutation is or comprises a modification in a codon of KIR2DL1 encoding amino acid G179 in the extracellular D2 domain.

18. The method of any one of claims 10-17, wherein the mutation in the extracellular D2 domain of KIR2DL1 is or comprises the G179R.

19. The method of any one of claims 10-18, wherein the mutation is or comprises a modification in a codon of KIR2DL1 encoding amino acid D184 in the extracellular D2 domain.

20. The method of any one of claims 10-19, wherein the mutation in the extracellular D2 domain of KIR2DL1 is or comprises the D184N.

21. The method of any one of claims 10-20, wherein the mutation is or comprises a modification in a codon of KIR2DL1 encoding amino acid R197 in the extracellular D2 domain.

22. The method of any one of claims 10-21, wherein the mutation in the extracellular D2 domain of KIR2DL1 is or comprises the R197T.

23. The method of any one of claims 10-22, wherein the mutation is or comprises a modification in a codon of KIR2DL1 encoding amino acid F202 in the extracellular D2 domain.

24. The method of any one of claims 10-23, wherein the mutation in the extracellular D2 domain of KIR2DL1 is or comprises the F202L.

25. The method of any one of claims 1-24, wherein the FTI, optionally tipifarnib, is selectively administered to a subject to treat the KIR-mutant cancer, and wherein the KIR-mutant cancer has or comprises a mutation in KIR2DL3.

26. The method of any one of claims 1-25, wherein the mutation is or comprises a modification in a codon of KIR2DL3 encoding an amino acid selected from a group consisting of: F66, R162, R169, F171, S172, E295, R318, I330, I331, and V332.

27. The method of any one of claims 1-26, wherein the mutation in the KIR2DL3 is selected from a group consisting of: F66Y, R162T, R169C, F171L, S172P, E295D, R318C, I330T, I331T, and V332M.

28. The method of any one of claims 1-27, wherein the mutation is or comprises modifications in two or more, or three or more, codons of KIR2DL3 encoding two or more, or three or more, amino acids selected from a group consisting of: F66, R162, R169, F171, S172, E295, R318, I330, I331, and V332.

29. The method of any one of claims 1-28, wherein the KIR2DL3 has two or more, or three or more, mutations selected from a group consisting of: F66Y, R162T, R169C, F171L, S172P, E295D, R318C, I330T, I331T, and V332M.

30. The method of any one of claims 26-29, wherein the mutation is or comprises a modification in a codon of KIR2DL3 encoding amino acid R162 and/or E295.

31. The method of any one of claims 26-30, wherein the mutation in the KIR2DL3 is or comprises the R162T and/or the E295D.

32. The method of any one of claims 1-31, wherein the mutation is or comprises a modification in a codon of KIR2DL3 encoding an amino acid in the extracellular D2 domain.

33. The method of claim 32, wherein the mutation is or comprises a modification in a codon of KIR2DL3 encoding an amino acid in the extracellular D2 domain selected from a group consisting of: F66, R162, R169, F171, and S172.

34. The method of any one of claims 32-33, wherein the mutation in the extracellular D2 domain of KIR2DL3 is selected from a group consisting of: F66Y, R162T, R169C, F171L, and S172P.

35. The method of any one of claims 32-34, wherein the mutation is or comprises a modification in a codon of KIR2DL3 encoding amino acid R162 in the extracellular D2 domain.

36. The method of any one of claims 32-35, wherein the mutation in the extracellular D2 domain of KIR2DL3 is or comprises the R162T.

37. The method of any one of claims 1-36, wherein the mutation is or comprises a modification in a codon of KIR2DL3 encoding an amino acid in the cytoplasmic domain.

38. The method of claim 37, wherein the mutation is or comprises a modification in a codon of KIR2DL3 encoding an amino acid in the cytoplasmic domain selected from a group consisting of: E295, R318, I330, I331, and V332.

39. The method of any one of claims 37-38, wherein the mutation in the cytoplasmic domain of KIR2DL3 is selected from a group consisting of: E295D, R318C, I330T, I331T, and V332M.

40. The method of any one of claims 37-39, wherein the mutation in the cytoplasmic domain of KIR2DL3 is within or near the CK2 site, the PKC site, and/or the immunoreceptor tyrosine-based inhibitory motif 2 (ITIM 2), of said cytoplasmic domain.

41. The method of claim 40, wherein the mutation in the cytoplasmic domain of KIR2DL3 is within or near the CK2 site of said cytoplasmic domain.

42. The method of any one of claims 40-41, wherein the mutation is or comprises a modification in a codon of KIR2DL3 encoding amino acid E295 positioned within or near the CK2 site of the cytoplasmic domain.

43. The method of any one of claims 40-42, wherein the mutation within or near the CK2 site of the cytoplasmic domain of KIR2DL3 is E295D.

44. The method of any one of claims 40-43, wherein the mutation in the cytoplasmic domain of KIR2DL3 is within or near the PKC site of said cytoplasmic domain.

45. The method of any one of claims 40-44, wherein the mutation is or comprises a modification in a codon of KIR2DL3 encoding amino acid R318 positioned within or near the PKC site of the cytoplasmic domain.

46. The method of any one of claims 40-45, wherein the mutation within or near the PKC site of the cytoplasmic domain of KIR2DL3 is R318C.

47. The method of any one of claims 40-46, wherein the mutation in the cytoplasmic domain of KIR2DL3 is within or near the ITIM 2 of said cytoplasmic domain.

48. The method of any one of claims 40-47, wherein the mutation is or comprises a modification in a codon of KIR2DL3 encoding an amino acid positioned within or near the ITIM 2 of the cytoplasmic domain selected from a group consisting of: I330, I331, and V332.

49. The method of any one of claims 40-48, wherein the mutation within or near the ITIM 2 of the cytoplasmic domain of KIR2DL3 is selected from a group consisting of: I330T, I331T, and V332M.

50. The method of any one of claims 37-49, wherein the mutation is or comprises modifications in two or more, or three or more, codons of KIR2DL3 encoding two or more, or three or more, amino acids in the cytoplasmic domain selected from a group consisting of: E295, R318, I330, I331, and V332.

51. The method of any one of claims 37-50, wherein the cytoplasmic domain of KIR2DL3 has two or more, or three or more, mutations selected from a group consisting of: E295D, R318C, I330T, I331T, and V332M.

52. The method of any one of claims 1-51, wherein the FTI, optionally tipifarnib, is selectively administered to a subject to treat the KIR-mutant cancer, and wherein the KIR-mutant cancer has or comprises a mutation in KIR2DL4.

53. The method of any one of claims 1-52, wherein the mutation is or comprises a modification in a codon of KIR2DL4 encoding an amino acid selected from a group consisting of: R50, H52, R55, N58, T61, K65, Q149, I154, E162, L166, I174, A238, and S267.

54. The method of any one of claims 1-53, wherein the mutation in the KIR2DL4 is selected from a group consisting of: R50L, H52R, R55L, N58T, T61R, K65E, Q149K, Q149R, I154M, E162K, E162G, L166P, I174V, A238P, and S267fs.

55. The method of any one of claims 1-54, wherein the mutation is or comprises modifications in two or more, or three or more, codons of KIR2DL4 encoding two or more, or three or more, amino acids selected from a group consisting of: R50, H52, R55, N58, T61, K65, Q149, I154, E162, L166, I174, A238, and S267.

56. The method of any one of claims 1-55, wherein the KIR2DL4 has two or more, or three or more, mutations selected from a group consisting of: R50L, H52R, R55L, N58T, T61R, K65E, Q149K, Q149R, I154M, E162K, E162G, L166P, I174V, A238P, and S267fs.

57. The method of any one of claims 1-56, wherein the mutation is or comprises a modification in a codon of KIR2DL4 encoding an amino acid in the extracellular domain.

58. The method of claim 57, wherein the mutation is or comprises a modification in a codon of KIR2DL4 encoding an amino acid in the extracellular domain selected from a group consisting of: R50, H52, R55, N58, T61, K65, Q149, I154, E162, L166, and I174.

59. The method of any one of claims 57-58, wherein the mutation in the extracellular domain of KIR2DL4 is selected from a group consisting of: R50L, H52R, R55L, N58T, T61R, K65E, Q149K, Q149R, I154M, E162K, E162G, L166P, and I174V.

60. The method of any one of claims 57-59, wherein the mutation is or comprises modifications in two or more, or three or more, codons of KIR2DL4 encoding two or more, or three or more, amino acids in the extracellular domain selected from a group consisting of: R50, H52, R55, N58, T61, K65, Q149, I154, E162, L166, and I174.

61. The method of any one of claims 57-60, wherein the extracellular domain of KIR2DL4 has two or more, or three or more, mutations selected from a group consisting of: R50L, H52R, R55L, N58T, T61R, K65E, Q149K, Q149R, I154M, E162K, E162G, L166P, and I174V.

62. The method of any one of claims 1-61, wherein the mutation is or comprises a modification in a codon of KIR2DL4 encoding an amino acid in the extracellular D2 domain.

63. The method of claim 62, wherein the mutation is or comprises a modification in a codon of KIR2DL4 encoding an amino acid in the extracellular D2 domain selected from a group consisting of: Q149, I154, E162, L166, and I174.

64. The method of any one of claims 62-63, wherein the mutation in the extracellular D2 domain of KIR2DL4 is selected from a group consisting of: Q149K, Q149R, I154M, E162K, E162G, L166P, and I174V.

65. The method of any one of claims 62-64, wherein the mutation is or comprises modifications in two or more, or three or more, codons of KIR2DL4 encoding two or more, or three or more, amino acids in the extracellular D2 domain selected from a group consisting of: Q149, I154, E162, L166, and I174.

66. The method of any one of claims 62-65, wherein the extracellular D2 domain of KIR2DL4 has two or more, or three or more, mutations selected from a group consisting of: Q149K, Q149R, I154M, E162K, E162G, L166P, and I174V.

67. The method of any one of claims 62-66, wherein the mutation is or comprises a modification in a codon of KIR2DL4 encoding amino acid Q149 and/or I154 in the extracellular D2 domain.

68. The method of any one of claims 62-67, wherein the mutation in the extracellular D2 domain of KIR2DL4 is or comprises the Q149K, Q149R, and/or I154M.

69. The method of any one of claims 62-68, wherein the mutation is or comprises a modification in a codon of KIR2DL4 encoding amino acid Q149 in the extracellular D2 domain.

70. The method of any one of claims 62-69, wherein the mutation in the extracellular D2 domain of KIR2DL4 is or comprises the Q149K and/or the Q149R.

71. The method of any one of claims 62-70, wherein the mutation is or comprises a modification in a codon of KIR2DL4 encoding amino acid I154 in the extracellular D2 domain.

72. The method of any one of claims 62-71, wherein the mutation in the extracellular D2 domain of KIR2DL4 is or comprises the I154M.

73. The method of any one of claims 1-72, wherein the mutation is or comprises a modification in a codon of KIR2DL4 encoding an amino acid in the cytoplasmic domain.

74. The method of claim 72, wherein the mutation is or comprises a modification in a codon of KIR2DL4 encoding an amino acid in the cytoplasmic domain selected from a group consisting of: A238 and S267.

75. The method of any one of claims 73-74, wherein the mutation in the cytoplasmic domain of KIR2DL4 is selected from a group consisting of: A238P and S267fs.

76. The method of any one of claims 73-75, wherein the mutation is or comprises a modification in a codon of KIR2DL4 encoding amino acid S267 in the cytoplasmic domain.

77. The method of any one of claims 72-76, wherein the mutation in the cytoplasmic domain of KIR2DL4 is or comprises the S267fs.

78. The method of any one of claims 1-77, wherein the FTI, optionally tipifarnib, is selectively administered to a subject to treat the KIR-mutant cancer, and wherein the KIR-mutant cancer has or comprises a mutation in KIR3DL1.

79. The method of any one of claims 1-78, wherein the mutation is or comprises a modification in a codon of KIR3DL1 encoding an amino acid selected from a group consisting of: R292, F297, P336, R409, R413, I426, L427, T429, and V440.

80. The method of any one of claims 1-79, wherein the mutation in the KIR3DL1 is selected from a group consisting of: R292T, F297L, P336R, R409T, R413C, I426T, L427M, T429M, and V440I.

81. The method of any one of claims 1-80, wherein the mutation is or comprises a modification in a codon of KIR3DL1 encoding an amino acid selected from a group consisting of: R292, F297, I426, L427, and T429.

82. The method of any one of claims 1-81, wherein the mutation in the KIR3DL1 is selected from a group consisting of: R292T, F297L, I426T, L427M, and T429M.

83. The method of any one of claims 1-82, wherein the mutation is or comprises modifications in two or more, or three or more, codons of KIR3DL1 encoding two or more, or three or more, amino acids selected from a group consisting of: R292, F297, P336, R409, R413, I426, L427, T429, and V440.

84. The method of any one of claims 1-83, wherein the KIR3DL1 has two or more, or three or more, mutations selected from a group consisting of: R292T, F297L, P336R, R409T, R413C, I426T, L427M, T429M, and V440I.

85. The method of any one of claims 1-84, wherein the mutation is or comprises modifications in two or more, or three or more, codons of KIR3DL1 encoding two or more, or three or more, amino acids selected from a group consisting of: R292, F297, I426, L427, and T429.

86. The method of any one of claims 1-85, wherein the KIR3DL1 has two or more, or three or more, mutations selected from a group consisting of: R292T, F297L, I426T, L427M, and T429M.

87. The method of any one of claims 1-86, wherein the mutation is or comprises a modification in a codon of KIR3DL1 encoding an amino acid in the extracellular domain.

88. The method of claim 87, wherein the mutation is or comprises a modification in a codon of KIR3DL1 encoding an amino acid in the extracellular domain selected from a group consisting of: R292, F297, and P336.

89. The method of any one of claims 87-88, wherein the mutation in the extracellular domain of KIR3DL1 is selected from a group consisting of: R292T, F297L, and P336R.

90. The method of any one of claims 87-89, wherein the mutation is or comprises modifications in two or more, or three or more, codons of KIR3DL1 encoding two or more, or three or more, amino acids in the extracellular domain selected from a group consisting of: R292, F297, and P336.

91. The method of any one of claims 87-90, wherein the extracellular domain of KIR3DL1 has two or more, or three or more, mutations selected from a group consisting of: R292T, F297L, and P336R.

92. The method of any one of claims 87-91, wherein the mutation is or comprises a modification in a codon of KIR3DL1 encoding amino acid R292 and/or F297 in the extracellular domain.

93. The method of any one of claims 87-92, wherein the mutation in the extracellular domain of KIR3DL1 is or comprises the R292T and/or the F297L.

94. The method of any one of claims 87-93, wherein the mutation is or comprises a modification in a codon of KIR3DL1 encoding amino acid R292 in the extracellular domain.

95. The method of any one of claims 87-94, wherein the mutation in the extracellular domain of KIR3DL1 is or comprises the R292T.

96. The method of any one of claims 87-95, wherein the mutation is or comprises a modification in a codon of KIR3DL1 encoding amino acid F297 in the extracellular domain.

97. The method of any one of claims 87-96, wherein the mutation in the extracellular domain of KIR3DL1 is or comprises the F297L.

98. The method of any one of claims 1-97, wherein the mutation is or comprises a modification in a codon of KIR3DL1 encoding an amino acid in the cytoplasmic domain.

99. The method of claim 98, wherein the mutation is or comprises a modification in a codon of KIR3DL1 encoding an amino acid in the cytoplasmic domain selected from a group consisting of: R409, R413, I426, L427, T429, and V440.

100. The method of any one of claims 98-99, wherein the mutation in the cytoplasmic domain of KIR3DL1 is selected from a group consisting of: R409T, R413C, I426T, L427M, T429M, and V440I.

101. The method of any one claims 98-100, wherein the mutation is or comprises a modification in a codon of KIR3DL1 encoding an amino acid in the cytoplasmic domain within or near the PKC site, the PDK site, and/or the immunoreceptor tyrosine-based inhibitory motif 2 (ITIM 2), of said cytoplasmic domain.

102. The method of any one of claims 98-101, wherein the mutation in the cytoplasmic domain of KIR3DL1 is within or near the PKC site of said cytoplasmic domain.

103. The method of any one of claims 101-102, wherein the mutation is or comprises a modification in a codon of KIR3DL1 encoding the amino acid R409 and/or R413 positioned within or near the PKC site of the cytoplasmic domain.

104. The method of any one of claims 101-103, wherein the mutation within or near the PKC site of the cytoplasmic domain of KIR3DL1 is or comprises R409T and/or R413C.

105. The method of any one of claims 101-104, wherein the mutation in the cytoplasmic domain of KIR3DL1 is within or near the ITIM 2 of said cytoplasmic domain.

106. The method of any one of claims 101-105, wherein the mutation within or near the ITIM 2 of the cytoplasmic domain of KIR3DL1 is or comprises an amino acid modification at a codon selected from a group consisting of: I426, L427, and T429.

107. The method of any one of claims 101-106, wherein the mutation within or near the ITIM 2 of the cytoplasmic domain of KIR3DL1 is selected from a group consisting of: I426T, L427M, and T429M.

108. The method of any one of claims 101-107, wherein the cytoplasmic domain of KIR3DL1 and within or near the ITIM 2 comprises two or more, or three or more, amino acid modifications at two or more, or three or more, codons selected from a group consisting of: I426, L427, and T429.

109. The method of any one of claims 101-108, wherein the cytoplasmic domain of KIR3DL1 and within or near the ITIM 2 has two or more, or three or more, mutations selected from a group consisting of: I426T, L427M, and T429M.

110. The method of any one of claims 101-109, wherein the mutation within or near the ITIM 2 of the cytoplasmic domain of KIR3DL1 is or comprises the amino acid modification at the codon I426.

111. The method of any one of claims 101-110, wherein the mutation within or near the ITIM 2 of the cytoplasmic domain of KIR3DL1 is or comprises the I426T.

112. The method of any one of claims 101-111, wherein the mutation within or near the ITIM 2 of the cytoplasmic domain of KIR3DL1 is or comprises the amino acid modification at the codon L427.

113. The method of any one of claims 101-112, wherein the mutation within or near the ITIM 2 of the cytoplasmic domain of KIR3DL1 is or comprises the L427M.

114. The method of any one of claims 101-113, wherein the mutation within or near the ITIM 2 of the cytoplasmic domain of KIR3DL1 is or comprises the amino acid modification at the codon T429.

115. The method of any one of claims 101-114, wherein the mutation within or near the ITIM 2 of the cytoplasmic domain of KIR3DL1 is or comprises the T429M.

116. The method of any one of claims 1-115, wherein the FTI, optionally tipifarnib, is selectively administered to a subject to treat the KIR-mutant cancer, and wherein the KIR-mutant cancer has or comprises a mutation in KIR3DL2.

117. The method of any one of claims 1-116, wherein the mutation is or comprises a modification in a codon of KIR3DL2 encoding an amino acid selected from a group consisting of: P319, W323, P324, S333, C336, V341, and Q386.

118. The method of any one of claims 1-117, wherein the mutation in the KIR3DL2 is selected from a group consisting of: P319S, W323S, P324S, S333T, C336R, V341I, and Q386E.

119. The method of any one of claims 1-118, wherein the KIR3DL2 comprises two or more, or three or more, amino acid modifications at two or more, or three or more, codons selected from a group consisting of: P319, W323, P324, S333, C336, V341, and Q386.

120. The method of any one of claims 1-119, wherein the KIR3DL2 has two or more, or three or more, mutations selected from a group consisting of: P319S, W323S, P324S, S333T, C336R, V341I, and Q386E.

121. The method of any one of claims 1-120, wherein the mutation in the KIR3DL2 is or comprises an amino acid modification at the codon C336 and/or Q386.

122. The method of any one of claims 1-121, wherein the mutation in the KIR3DL2 is or comprises the C336R and/or the Q386E.

123. The method of any one of claims 1-122, wherein the mutation is or comprises a modification in a codon of KIR3DL2 encoding an amino acid in the extracellular domain.

124. The method of claim 123, wherein the mutation is or comprises a modification in a codon of KIR3DL2 encoding an amino acid in the extracellular domain selected from a group consisting of: P319, W323, P324, S333, C336, and V341.

125. The method of any one of claims 123-124, wherein the mutation in the extracellular domain of KIR3DL2 is selected from a group consisting of: P319S, W323S, P324S, S333T, C336R, and V341I.

126. The method of any one of claims 123-125, wherein the mutation in the extracellular domain of KIR3DL2 is or comprises an amino acid modification at the codon C336.

127. The method of any one of claims 123-125, wherein the mutation in the extracellular domain of KIR3DL2 is or comprises the C336R.

128. The method of any one of claims 1-127, wherein the mutation is or comprises a modification in a codon of KIR3DL2 encoding an amino acid in the cytoplasmic domain.

129. The method of claim 128, wherein the mutation in the cytoplasmic domain of KIR3DL2 is an amino acid modification at codon Q386.

130. The method of any one of claims 128-129, wherein the mutation in the cytoplasmic domain of KIR3DL2 is Q386E.

131. The method of any one of claims 1-130, wherein the KIR-mutant cancer is a cancer known to have or determined to have a mutation in two or more members of the KIR family selected from the group consisting of: KIR2DL1, KIR2DL3, KIR2DL4, KIR3DL1, and KIR3DL2.

132. The method of any one of claims 1-131, wherein the KIR-mutant cancer is a cancer known to have or determined to have a mutation in KIR2DL3 and KIR3DL2.

133. The method of claim 132, wherein the mutation in the KIR2DL3 is or comprises the amino acid modification at codon R162 and/or E295, and wherein the mutation in the KIR3DL2 is or comprises an amino acid modification at codon C336 and/or Q386.

134. The method of any one of claims 132-133, wherein the mutation in the KIR2DL3 is or comprises R162T and/or E295D, and wherein the mutation in the KIR3DL2 is or comprises C336R and/or Q386E.

135. The method of any one of claims 132-134, wherein the mutation in the KIR2DL3 is or comprises the amino acid modification at codon R162.

136. The method of any one of claims 132-135, wherein the mutation in the KIR2DL3 is or comprises R162T.

137. The method of any one of claims 132-136, wherein the mutation in the KIR2DL3 is or comprises the amino acid modification at codon E295.

138. The method of any one of claims 132-137, wherein the mutation in the KIR2DL3 is or comprises E295D.

139. The method of any one of claims 132-138, wherein the mutation in the KIR3DL2 is or comprises an amino acid modification at codon C336.

140. The method of any one of claims 132-139, wherein the mutation in the KIR3DL2 is or comprises C336R.

141. The method of any one of claims 132-140, wherein the mutation in the KIR3DL2 is or comprises an amino acid modification at codon Q386.

142. The method of any one of claims 132-141, wherein the mutation in the KIR3DL2 is or comprises Q386E.

143. The method of any one of claims 1-142, wherein the method comprises determining a KIR-mutant cancer variant allele frequency (VAF) in a sample from the subject, wherein the KIR-mutant cancer is selected from the group consisting of: a KIR2DL1-mutant, a KIR2DL3-mutant, a KIR2DL4-mutant, a KIR3DL1-mutant, and/or a KIR3DL2-mutant.

144. The method of claim 143, wherein the KIR-mutant VAF is determined by sequencing, Next Generation Sequencing (NGS), Polymerase Chain Reaction (PCR), DNA microarray, Mass Spectrometry (MS), Single Nucleotide Polymorphism (SNP) assay, denaturing high-performance liquid chromatography (DHPLC), or Restriction Fragment Length Polymorphism (RFLP) assay.

145. The method of claim 144, wherein the KIR-mutant VAF is determined by sequencing, Next Generation Sequencing (NGS).

146. The method of any one of claims 1-145, wherein the KIR-mutant cancer is a cancer known to have or determined to have a mutation in three or more members of the KIR family selected from the group consisting of: KIR2DL1, KIR2DL3, KIR2DL4, KIR3DL1, and KIR3DL2.

147. The method of any one of claims 1-146, wherein the KIR-mutant cancer is a hematological cancer or hematopoietic cancer.

148. The method of any one of claims 1-147, wherein the KIR-mutant cancer is a lymphoma, leukemia, myelodysplastic syndrome (MDS), or myeloproliferative neoplasm (MPN).

149. The method of any one of claims 1-148, wherein the KIR-mutant cancer is a lymphoma.

150. The method of claim 149, wherein the lymphoma is a natural killer cell lymphoma (NK lymphoma).

151. The method of claim 150, wherein the lymphoma is a natural killer cell leukemia (NK leukemia).

152. The method of claim 150, wherein the lymphoma is a cutaneous T-Cell lymphoma (CTCL).

153. The method of claim 150, wherein the lymphoma is a peripheral T-cell lymphoma (PTCL).

154. The method of claim 153, wherein the PTCL is relapsed or refractory PTCL.

155. The method of claim 153, wherein the PTCL is PTCL not otherwise specified (PTCL-NOS).

156. The method of claim 155, wherein the PTCL-NOS is relapsed or refractory PTCL-NOS.

157. The method of claim 153, wherein the PTCL is angioimmunoblastic T-cell lymphoma (AITL).

158. The method of claim 157, wherein the AITL has a KIR3DL2 C336R mutation variant allele frequency (VAF) of greater than 10%.

159. The method of claim 158, wherein the KIR3DL2 C336R mutation VAF is greater than 15% or greater than 20%.

160. The method of any one of claims 157-159, wherein the AITL has a KIR3DL2 Q386E mutation variant allele frequency (VAF) of greater than 5%.

161. The method of claim 160, wherein the KIR3DL2 Q386E mutation VAF is greater than 6%, greater than 7%, greater than 8%, or greater than 9%.

162. The method of any one of claims 157-161, wherein the AITL is relapsed or refractory AITL.

163. The method of claim 153, wherein the PTCL is AITL not otherwise specified (AITL-NOS).

164. The method of claim 163, wherein the AITL-NOS is relapsed or refractory AITL-NOS.

165. The method of any one of claims 153-164, wherein the FTI, optionally tipifarnib, is selectively administered to the subject on the basis that the subject has a tumor of AITL histology.

166. The method of claim 165, wherein the AITL histology is characterized by a tumor cell component.

167. The method of claim 166, wherein the tumor cell component comprises polymorphous medium-sized neoplastic cells derived from follicular helper T cells.

168. The method of any one of claims 165-167, wherein the AITL histology is characterized by a non-tumor cell component.

169. The method of claim 168, wherein the non-tumor cell component comprises prominent arborizing blood vessels.

170. The method of any one of claims 168-169, wherein the non-tumor cell component comprises proliferation of follicular dendritic cells.

171. The method of any one of claims 168-170, wherein the non-tumor cell component comprises scattered EBV positive B-cell blasts.

172. The method of any one of claim 157-162 or 165-171, wherein the subject having AITL has been diagnosed with AITL.

173. The method of claim 172, wherein diagnosis with AITL comprises visualization of a non-tumor component.

174. The method of claim 172, wherein diagnosis with AITL comprises visualization of proliferation of endothelial venules.

175. The method of claim 172, wherein the AITL is refractory and resistant to a prior standard of care (SOC) treatment selected from the group consisting of: Nivolumab, BEAM/ASCT, DICE, CHOP-E, Brentuximab ved., CEOP, and GemDOX.

176. The method of claim 173, wherein the refractory and resistant AITL has a KIR3DL2 Q386E mutation VAF of greater than 5%.

177. The method of claim 174, wherein the subject has an improved overall response rate to tipifarnib administration relative to the overall response rate of the prior SOC treatment.

178. The method of any one of claims 163-171, wherein the subject having AITL-NOS has been diagnosed with AITL-NOS.

179. The method of claim 178, wherein diagnosis with AITL-NOS comprises visualization of a non-tumor component.

180. The method of claim 178, wherein diagnosis with AITL-NOS comprises visualization of proliferation of endothelial venules.

181. The method of claim 153, wherein the PTCL is anaplastic large cell lymphoma (ALCL)-anaplastic lymphoma kinase (ALK) positive.

182. The method of claim 153, wherein the PTCL is anaplastic large cell lymphoma (ALCL)-anaplastic lymphoma kinase (ALK) negative.

183. The method of claim 153, wherein the PTCL is enteropathy-associated T-cell lymphoma.

184. The method of claim 153, wherein the PTCL is extranodal natural killer cell (NK) T-cell lymphoma—nasal type.

185. The method of claim 153, wherein the PTCL is hepatosplenic T-cell lymphoma.

186. The method of claim 153, wherein the PTCL is subcutaneous panniculitis-like T-cell lymphoma.

187. The method of claim 149, wherein the lymphoma is an EBV associated lymphoma.

188. The method of claim 149, wherein the lymphoma is a T-cell lymphoma.

189. The method of any one of claims 1-148, wherein the KIR-mutant cancer is a leukemia.

190. The method of claim 189, wherein the leukemia is acute myeloid leukemia (AML).

191. The method of claim 190, wherein the AML is newly diagnosed AML.

192. The method of any one of claims 190-191, wherein the subject having AML is either an elderly patient, unfit for chemotherapy, or with poor-risk AML.

193. The method of any one of claims 190-192, wherein the AML is relapsed or refractory AML.

194. The method of claim 189, wherein the leukemia is T-cell acute lymphoblastic leukemia (T-ALL).

195. The method of claim 189, wherein the leukemia is chronic myelogenous leukemia (CML).

196. The method of claim 189, wherein the leukemia is chronic myelomonocytic leukemia (CMML).

197. The method of claim 189, wherein the leukemia is juvenile myelomonocytic leukemia (JMML).

198. The method of any one of claims 1-148, wherein the KIR-mutant cancer is a myelodysplastic syndrome (MDS) or myeloproliferative neoplasms (MPN).

199. The method of claim 198, wherein the MDS or MPN is CMML.

200. The method of claim 198, wherein the MDS or MPN is JMML.

201. The method of any one of claims 1-200, comprising a step of detecting the presence of a mutation in a member of the KIR family in a sample from said subject.

202. The method of claim 201, wherein said sample is a bone marrow sample or a plasma sample.

203. The method of any one of claims 201-202, wherein the mutation is detected by a method selected from the group consisting of sequencing, Polymerase Chain Reaction (PCR), DNA microarray, Mass Spectrometry (MS), Single Nucleotide Polymorphism (SNP) assay, denaturing high-performance liquid chromatography (DHPLC), and Restriction Fragment Length Polymorphism (RFLP) assay.

204. The method of any one of claims 201-203, wherein the sample is a cell or tissue of the KIR-mutant cancer, and wherein the KIR-mutant cancer is determined to have a mutation in a member of the KIR family.

205. The method of any one of claims 1-204, wherein the subject is responsive to treatment for at least or more than 3 months, 4 months, 5 months, 6 months, 7 months, 8 months, 9 months or 1 year.

206. The method of any one of claims 1-205, wherein the administering is performed for at least or more than 3 months, 4 months, 5 months, 6 months, 7 months, 8 months, 9 months or 1 year.

207. The method of any one of claims 1-206, wherein the FTI, optionally tipifarnib, is administered orally, parenterally, rectally, or topically.

208. The method of any one of claims 1-207, wherein the FTI, optionally tipifarnib, is administered at a dose of 0.05-500 mg/kg body weight.

209. The method of any one of claims 1-208, wherein the FTI, optionally tipifarnib, is administered twice a day.

210. The method of any one of claims 1-209, wherein the FTI, optionally tipifarnib, is administered at a dose of 200-1200 mg twice a day.

211. The method of claim 210, wherein the FTI, optionally tipifarnib, is administered at a dose of 100 mg, 200 mg, 300 mg, 400 mg, 600 mg, 900 mg or 1200 mg twice a day.

212. The method of any one of claims 1-211, wherein the FTI, optionally tipifarnib, is administered on days 1-7 and 15-21 of a 28-day treatment cycle.

213. The method of any one of claims 1-211, wherein the FTI, optionally tipifarnib, is administered on days 1-21 of a 28-day treatment cycle.

214. The method of any one of claims 1-211, wherein the FTI, optionally tipifarnib, is administered on days 1-7 of a 28-day treatment cycle.

215. The method of any one of claims 212-214, wherein the FTI, optionally tipifarnib, is administered for at least 1 cycle.

216. The method of any one of claims 209-215, wherein the FTI, optionally tipifarnib, is administered at a dose of 900 mg twice a day

217. The method of any one of claims 209-215, wherein the FTI, optionally tipifarnib, is administered at a dose of 600 mg twice a day.

218. The method of any one of claims 209-215, wherein the FTI, optionally tipifarnib, is administered at a dose of 400 mg twice a day

219. The method of any one of claims 209-215, wherein the FTI, optionally tipifarnib, is administered at a dose of 300 mg twice a day.

220. The method of any one of claims 209-215, wherein the FTI, optionally tipifarnib, is administered at a dose of 200 mg twice a day.

221. The method of any one of claims 1-220, wherein the FTI, optionally tipifarnib, is administered before, during, or after radiation.

222. The method of any one of claims 1-221, wherein the FTI is tipifarnib.

223. The method of any one of claims 1-222, further comprising administering a therapeutically effective amount of a second active agent or a support care therapy.

224. The method of claim 223, wherein the second active agent is a histone deacetylase, an antifolate, or chemotherapy.
